(19) United States
(12) Patent Application Publication
(10) Pub. No.: US 2019/0284642 A1
(43) Pub. Date: Sep. 19, 2019

(54) BIOMARKERS FOR THE PROGNOSIS AND DIAGNOSIS OF CANCER

(71) Applicant: QUEEN MARY UNIVERSITY OF LONDON, London (GB)

(72) Inventors: Frances Balkwill, London (GB); Martin Knight, London (GB); Conrad Bessant, London (GB); Oliver Pearce, London (GB); Robin Delaine-Smith, London (GB); Eleni Maniati, London (GB); Jun Wang, London (GB)

(21) Appl. No.: 16/462,990

(22) PCT Filed: Nov. 23, 2017
(86) PCT No.: PCT/EP17/80281

§ 371 Date: May 22, 2019

(30) Foreign Application Priority Data
Nov. 23, 2016 (GB) 1619808.7

(51) Int. Cl. C12Q 1/6886; C12Q 1/6806

(52) U.S. Cl. CPC C12Q 1/6886; C12Q 2600/158; C12Q 2600/118; C12Q 1/6806

(57) ABSTRACT

The present invention relates to biomarkers and biomarker panels useful in the prognosis and diagnosis of cancers, in particular epithelial cancers. The present invention also provides methods of treatment of patients diagnosed or having undergone diagnosis or prognosis using the biomarkers and biomarker panels of the invention. Kits for the analysis of the biomarkers and biomarker panels are also provided. The biomarker panel consists of COL11A1, CTS, ANXA6, LGALS3, ANXA1, AB13BP, COMP, COL1A1, LAMB1, CTSG, LAMA4, TNXB, FN1, AGT, FBLN2, HSPG2, COL6A6, VCAN, ANXA5, LAMC1, COL15A1 and VWF.

The present invention relates to biomarkers and biomarker panels useful in the prognosis and diagnosis of cancers. The present invention also provides methods of treatment of patients diagnosed or having undergone diagnosis or prognosis using the biomarkers and biomarker panels of the invention. Kits for the analysis of the biomarkers and biomarker panels are also provided.

### BACKGROUND

Solid tumors consist of malignant cells surrounded and infiltrated by a variety of non-malignant cells that are recruited and ‘corrupted’ by the cancer cells, aiding growth and spread. A dynamic network of soluble factors, cytokines, chemokines, growth factors and adhesion molecules drive the interactions between malignant and non-malignant cells to create this tumor microenvironment (TME). The TME network stimulates extracellular matrix (ECM) remodeling, expansion of the vascular and lymphatic networks and migration of cells into and out of the tumor mass. Solid tumors are also typically stiffer than the surrounding tissue due to abnormal ECM deposition that has a major influence on cell and tissue mechanics.

While the TME is of critical importance during initiation and spread of cancer, relatively little is known about its evolution or the relationship between the molecular mechanisms of disease progression and higher-order features such as tissue stiffness, extent of disease and cellularity. Studies on molecular mechanisms of human cancer have mainly focused on large scale genomic and transcriptomic analysis of primary tumors and the immune cell landscape. Human cancer evolution is also now being studied in multiple metastatic sites but mainly in terms of the genomics of the malignant cells.

Using multi-layered TME profiling of evolving omental metastases of high-grade serous ovarian cancer (HGSOC), the aims of the inventors were to identify molecular changes that predict the higher-order features and to provide a template for bioengineering complex 3D TME models. HGSOC is one of the most lethal of the peritoneal cancers: less than 30% of patients currently survive more than five years after diagnosis with little improvement in overall survival in the past 40 years. Poor prognosis is mainly due to early dissemination into the peritoneal cavity. HGSOC has a complex TME but there is little integrated understanding of its different components. The inventors chose to study the omental TME because it is the most frequent site for HGSOC tumor deposits and is routinely resected during debulking surgery.

Using samples ranging from normal to heavily diseased, the inventors conducted molecular, cellular and biomechanical analyses on each biopsy and used multivariate analyses to integrate the different components. This allowed the present inventors to define for the first time gene and protein profiles that predicted tissue stiffness, extent of disease and cellularity and to define how the entire ECM is remodeled during tumor development. Of particular interest was an ECM-associated molecular signature that predicted both tissue architecture and stiffness. This novel matrix signature distinguished patients with shorter overall survival not only in ovarian cancer, but also in at least twelve other cancer types irrespective of patient age, stage or response to primary treatment, suggesting a common matrix response to human primary and metastatic cancers that can be used to diagnose and prognose patients.

### SUMMARY OF THE INVENTION

The inventors have surprisingly found that certain ECM-associated genes are prognostic and diagnostic for a range of cancers. These biomarker genes correlate with higher order features of the tumour microenvironment during development of metastases, such as tissue stiffness, architecture and cellularity, to provide a prognosis for cancers, particularly epithelial cancers, such as ovarian cancer. The genes are part of the tissue matrisome, which is the esemble of ECM proteins and associated factors.

The novel ECM-associated signature is a previously unknown common matrix response to human cancers, and demonstrates the biomarkers and biomarker panels of the present invention are of prognostic and diagnostic significance for a range of cancers. The biomarkers and biomarker panels also present a potential for targeting treatment to a consistent feature of many cancers.

In a first aspect of the invention, there is provided a method of diagnosing or prognosing cancer, comprising measuring, in a patient sample, the expression of at least two of the genes selected from the group consisting of COL11A1, CTS, ANXA6, LGALS3, ANXA1, AB13BP, COMP, COL1A1, LAMB1, CTSG, LAMA4, TNXB, FN1, AGT, FBLN2, HSPG2, COL6A6, VCAN, ANXA5, LAMC1, COL15A1 and VWF. In some embodiments of the invention, the biomarker panel comprises CTSB and LAMC1. In preferred embodiments, the biomarker panel comprises CTSB; at least one gene selected from the group consisting of COL11A1, COMP, FN1, VCAN and COL1A1; at least one gene selected from the group consisting of LGALS3, AGT and ANXA6; at least one gene selected from the group consisting of COL6A6, AB13BP, TNXB, LAMB1, CTSG and LAMA4; LAMC1; and at least one gene selected from the group consisting of ANXA5, ANXA1, FBLN2, HSPG2, COL15A1 and VWF. In a more preferred embodiment, the biomarker panel comprises at least one gene selected from the group consisting of COL11A1, COMP, FN1, VCAN, CTSB and COL1A1 and at least one gene selected from the group consisting of ANXA6, LGALS3, ANXA1, AB13BP, LAMB1, CTSG, LAMA4, TNXB, AGT, FBLN2, HSPG2, COL6A6, ANXA5, LAMC1, COL15A1 and VWF. In a further preferred embodiment, the biomarker panel comprises COL11A1, ANXA6, LAMC1, CTSB, LAMA4 and HSPG2.

The methods of the invention may use tissue samples and comprise the determination of an expression profile of the biomarker proteins or genes in the tissue sample.

In a second aspect of the invention, there is provided a method of predicting metastases, or identifying patients with a poor prognosis, comprising measuring the expression of at least two genes of the biomarker panels of the invention. The methods may comprise determining a quantitative expression ratio between these two genes. In some embodiments, the methods comprise determining a quantitative ratio between two groups of genes selected from the biomarker panels.

In a third aspect of the invention, there is provided a method of treating cancer in a patient in need thereof, comprising administering a cancer therapy or initiating a therapeutic regimen for cancer to the patient if cancer is diagnosed or suspected, or if cancer metastasis is predicted or a poor prognosis is suspected, wherein the cancer has been diagnosed or prognosed according to a method of diagnosis or prognosis of the invention. In some embodiments, the methods of treatment comprise the steps of diagnosing or prognosing the cancer according to a method of diagnosis or prognosis of the invention.

In a fourth aspect of the invention, there is provided a kit for the diagnosis or prognosis cancer, comprising means for measuring at least two genes of the biomarker panels of the invention.

In a fifth aspect of the invention, there is provided a method of determining a treatment regimen for a cancer patient for a patient suspected of having cancer, or for a patient having a poor prognosis, comprising:
(i) providing or obtaining a sample from a patient;
(ii) optionally enriching the sample for protein or RNA and/or extracting protein or RNA from the sample;
(iii) diagnosing or prognosing cancer according to a method of diagnosis or prognosis of the invention;
(iv) selecting a treatment regimen for the patient according to the presence or absence cancer as determined in step (iii).

In a further aspect of the invention, there is provided a method of predicting a patient's responsiveness to a cancer treatment, comprising
(i) providing or obtaining a sample from a patient;
(ii) optionally enriching the sample for protein or RNA and/or extracting protein or RNA from the sample;
(iii) diagnosing or prognosing cancer according to a method of the invention;
(iv) predicting a patient's responsiveness to a cancer treatment according to the presence or absence of cancer as determined in step (iii).

In a still further aspect of the invention, there is provided a microarray, comprising specific binding molecules that hybridize to an expression product from at least two genes of the biomarker panels of the invention.

### BRIEF DESCRIPTION OF THE FIGURES

FIG. 1. Study design and sample description

FIG. 2. Identification of molecular components that define tissue modulus

FIG. 3. Identification of ECM proteins and genes that define tissue architecture

FIG. 4. The cells of the TME change with disease score and tissue modulus

FIG. 5. Development of a matrix signature that predicts survival in ovarian cancer.

FIG. 6. Matrix index reveals a common stromal reaction across cancers

FIG. 7. Distribution of matrix index (22 genes) across cancer datasets

FIG. 8. Distribution of matrix index (6 genes) across cancer datasets

FIG. 9. Prediction of cancer survival in various cancers using the 22 gene matrix index

FIG. 10. Prediction of cancer survival in various cancers using the 6 gene matrix index

FIG. 11. Comparison of prognostic signatures using TCGA OV u133a dataset

FIG. 12. Correlation of matrix index—6 with disease score and tissue modulus still significant and close to matrix index—22

FIG. 13. Overview of the biomechanical approach taken to quantify tissue modulus.

FIG. 14. Analysis used to identify components associated with tissue modulus.

FIG. 15. Analysis of PLS-identified ECM proteins and genes. a)

FIG. 16. Immune cells and cytokines of the tumor microenvironment

FIG. 17. The matrix index signature

FIG. 18. The matrix index in other cancers

### DETAILED DESCRIPTION OF THE INVENTION

The present invention relates to prognosis and diagnosis of cancer, in particular epithelial cancers, by determining the expression profile of a set of genes in a sample derived from the tumour microenvironment.

### Biomarkers and Biomarker Panels of the Invention

The present invention provides several biomarkers (genes) and in particular biomarker panels that are useful in the prognosis and diagnosis of cancers.

In some embodiments of the invention, the biomarker panel is panel 1:

Panel 1

| | | | | | |
|---|---|---|---|---|---|
| COL11A1 | CTSB | ANXA6 | LGALS3 | ANXA1 | AB13BP |
| COMP | COL1A1 | LAMB1 | CTSG | LAMA4 | TNXB |
| FN1 | AGT | FBLN2 | HSPG2 | COL6A6 | |
| VCAN | ANXA5 | LAMC1 | COL15A1 | VWF | |

Further details of the biomarkers are provided below.

| | | | | HUGO Gene | | |
|---|---|---|---|---|---|---|
| | Gene | | | Nomenclature | | |
| Ensembl IDs | Name | Description | Synonyms | Committee IDs | UniProt IDs | Refseq IDs |
| ENSG00000105664.10 | COMP | cartilage | EDM1\|EPD1\|MED\|MGC131819\| | 2227 | B4DKJ3:G3XAP6:P49747 | NP_000086.2 |
| | | oligomeric | MGC149768\|PSACH\|THBS5 | | | |
| | | matrix protein | | | | |
| ENSG00000115414.18 | FN1 | fibronectin 1 | CIG\|DKFZp686F10164\|DKFZp686H0342\| | 3778 | F8W7G7:H0Y4K8:H0Y7Z1: | NP_002017.1:NP_473375.2:NP_997639.1:NP_997641.1: |
| | | | DKFZp686I1370\|DKF | | P02751 | NP_997643.1:NP_997647.1:XP_005246457.1: |
| | | | | | | XP_005246463.1:XP_005246470.1: |
| | | | | | | XP_005246472.1:XP_005246474.1 |
| ENSG00000038427.15 | VCAN | versican | CSPG2\|DKFZp686K06110\|ERVR\| | 2464 | D6RGZ6:E9PF17:P13611: | NP_001119808.1:NP_001119808.1:NP_001157569.1: |
| | | | GHAP\|PG-M\|WGN\|WGN1 | | Q86W61 | NP_001157570.1:NP_004376.2 |
| ENSG00000060718.19 | COL11A1 | collagen type | CO11A1\|COLL6\|STL2 | 2186 | C9JMN2:H7C381:P12107 | NP_001177638.1:NP_001845.3:NP_542196.2: |
| | | XI alpha 1 | | | | NP_542197.3 |
| | | chain | | | | |
| ENSG00000108821.13 | COL1A1 | collagen type I | OI4 | 2197 | I3L3H7:P02452 | NP_000079.2 |
| | | alpha 1 | | | | |
| ENSG00000164733.20 | CTSB | cathepsin B | APPS\|CPSB | 2527 | E9PCB3:E9PHZ5:E9PID0: | NP_001899.1:NP_680090.1:NP_680091.1:NP_680092.1: |
| | | | | | E9PIS1:E9PJ67:E9PKQ7: | NP_680093.1:XP_006716307.1: |
| | | | | | E9PKX0:E9PL32:E9PLY3: | XP_006716308.1 |
| | | | | | E9PNL5:E9PQM1:E9PR00: | |
| | | | | | E9PR54:E9PS78: | |
| | | | | | E9PSG5:P07858:R4GMQ5 | |
| ENSG00000131981.15 | LGALS3 | lectin, | CBP35\|GAL3\|GALBP\|GALIG\|LGALS2\| | 6563 | G3V3R6:G3V407:P17931 | NP_002297.2 |
| | | galactoside | MAC2 | | | |
| | | binding | | | | |
| | | soluble 3 | | | | |
| ENSG00000135744.7 | AGT | angiotensinogen | ANHU\|FLJ92595\|FLJ97926\|SERPINA8 | 333 | P01019 | NP_000020.1 |
| ENSG00000197043.13 | ANXA6 | annexin A6 | ANX6\|CBP68 | 544 | A6NN80:E5RFF0:ESRI05: | NP_001146.2:NP_001180473.1:XP_005268489.1 |
| | | | | | E5RIU8:E5RJF5:E5RJR0: | |
| | | | | | E5RK63:E5RK69:E7EMC6: | |
| | | | | | H0YC77:P08133 | |
| ENSG00000206384.10 | COL6A6 | collagen type | — | 27023 | A6NMZ7:F8W6Y7:H0Y940: | NP_001096078.1:XP_005247178.1 |
| | | VI alpha 6 | | | H0YA33 | |
| ENSG00000154175.16 | ABI3BP | ABI family | FLJ41743\|FLI41754\|NESHBP\|TARSH | 17265 | B4DSV9:D3YTG3:E9PPR9: | NP_056244.2:XP_005247340.1 |
| | | member 3 | | | E9PRB5:H0Y897:H0YCG4: | |
| | | binding | | | H0YCP4:H0YDN0: | |
| | | protein | | | H0YDW0:H0YEA0:H0YEL2: | |
| | | | | | H0YF18:H0YF57:H7C4H3: | |
| | | | | | H7C4N5:H7C4S3: | |
| | | | | | H7C4T1:H7C4X4:H7C524: | |
| | | | | | H7C556:H7C5S3:Q5JPC9: | |
| | | | | | Q7Z7G0 | |
| ENSG00000168477.17 | TNXB | tenascin XB | HXBL\|TENX\|TNX\|TNXB1\|TNXB2\| | 11976 | C9J7W4:E7EPZ9:P22105 | NP_061978.6:NP_115859.2 |
| | | | TNXBS\|XB\|XBS | | | |
| ENSG00000091136.13 | LAMB1 | laminin | CLM\|MGC142015 | 6486 | C9J296:E7EPA6:E9PCS6: | NP_002282.2 |
| | | subunit beta 1 | | | G3XAI2:P07942 | |
| ENSG00000112769.18 | LAMA4 | laminin | CLM\|MGC142015 | 6486 | C9J296:E7EPA6:E9PCS6: | NP_002282.2 |
| | | subunit alpha 4 | | | G3XAI2:P07942 | |
| ENSG00000100448.3 | CTSG | cathepsin G | CG\|MGC23078 | 2532 | P08311 | NP_001902.1 |
| ENSG00000135862.5 | LAMC1 | laminin | LAMB2\|MGC87297 | 6492 | P11047:R4GNC7 | NP_002284.3 |
| | | subunit | | | | |
| | | gamma 1 | | | | |
| ENSG00000135046.13 | ANXA1 | annexin A1 | ANX1\|LPC1 | 533 | P04083:Q5T3N0:Q5T3N1 | NP_000691.1 |
| ENSG00000164111.14 | ANXA5 | annexin A5 | ANX5\|ENX2\|PP4 | 543 | D6RBE9:D6RBL5:D6RCN3: | NP_001145.1 |
| | | | | | E9PHT9:P08758 | |
| ENSG00000110799.13 | VWF | von | F8VWF\|VWD | 12726 | I3L4K4:P04275:Q8TCE8 | NP_000543.2 |
| | | Willebrand | | | | |
| | | factor | | | | |
| ENSG00000204291.10 | COL15A1 | collagen type | FLJ38566 | 2192 | P39059 | NP_001846.3 |
| | | XV alpha 1 | | | | |
| | | chain | | | | |
| ENSG00000142798.17 | HSPG2 | heparan | PLC\|PRCAN\|SJA\|SJS\|SJS1 | 5273 | H0Y5A9:H7BYA5:H7C4A6: | NP_001278789.1:NP_005520.4 |
| | | sulfate | | | P98160:Q5SZI5:Q5SZI9: | |
| | | proteoglycan 2 | | | Q5SZJ1:Q5SZJ2 | |
| ENSG00000163520.13 | FBLN2 | fibulin 2 | — | 3601 | C9JQS6:F5H1F3:H7BXL0: | NP_001004019.1:NP_001158507.1:NP_001989.2 |
| | | | | | H7C1A3:P98095 | |

It is not necessary to use all of the biomarkers of the panel. For example, the invention may comprise the use of at least 2, at least 3, at least 4, at least 5, at least 6, at least 7, at least 8, at least 9, at least 10, or at least 15 of the biomarkers of panel 1. In a preferred embodiment, the invention comprises the use of at least two biomarkers of panel 1. For example, in a preferred embodiment, the invention comprises the use of at least one gene selected from the group consisting of COL11A1, COMP, FN1, VCAN, CTSB and COL1A1 and at least one gene selected from the group consisting of ANXA6, LGALS3, ANXA1, AB13BP, LAMB1, CTSG, LAMA4, TNXB, AGT, FBLN2, HSPG2, COL6A6, ANXA5, LAMC1, COL15A1 and VWF.

In a more preferred embodiment, the invention comprises the use of at least 6 biomarkers of panel 1.

For example, the present inventors have surprisingly discovered that the biomarker panels comprising at least 6 biomarkers, wherein one biomarker is selected from each of groups 1 to 6 shown below, are particularly useful in the prognosis and diagnosis of cancer:

Panel 2

| Group 1 | Group 2 | Group 3 | Group 4 | Group 5 | Group 6 |
|---|---|---|---|---|---|
| CTSB | COL11A1 | LGALS3 | COL6A6 | LAMC1 | ANXA5 |
| | COMP | AGT | AB13BP | | ANXA1 |
| | FN1 | ANXA6 | TNXB | | FBLN2 |
| | VCAN | | LAMB1 | | HSPG2 |
| | COL1A1 | | CTSG | | COL15A1 |
| | | | LAMA4 | | VWF |

For example, in some embodiments of the invention, the invention may comprise the use of the biomarkers of panel 3:

Panel 3

| | | |
|---|---|---|
| COL11A1 | ANXA6 | LAMC1 |
| CTSB | LAMA4 | HSPG2 |

The present invention also provides the combination of at least two of the genes selected from the group consisting of COL11A1, CTS, ANXA6, LGALS3, ANXA1, AB13BP, COMP, COL1A1, LAMB1, CTSG, LAMA4, TNXB, FN1, AGT, FBLN2, HSPG2, COL6A6, VCAN, ANXA5, LAMC1, COL15A1 and VWF for use in the diagnosis or prognosis of cancer. In some embodiments of the invention, the invention provides the combination of at least 2, at least 3, at least 4, at least 5, at least 6, at least 7, at least 8, at least 9, at least 10, or at least 15 of the biomarkers of panel 1 for use in the diagnosis or prognosis of cancer. In a preferred embodiment, the invention provides the combination of at least 6 genes of panel 1 for use in the diagnosis or prognosis of cancer. In another preferred embodiment, the invention provides the combination of at least one gene selected from the group consisting of COL11A1, COMP, FN1, VCAN, CTSB and COL1A1 and at least one gene selected from the group consisting of ANXA6, LGALS3, ANXA1, AB13BP, LAMB1, CTSG, LAMA4, TNXB, AGT, FBLN2, HSPG2, COL6A6, ANXA5, LAMC1, COL15A1 and VWF for use in the diagnosis or prognosis of cancer. In a more preferred embodiment, the invention provides the combination of COL11A1, ANXA6, LAMC1, CTSB, LAMA4 and HSPG2 for use in the diagnosis or prognosis of cancer. The present invention also provides the use of biomarker panels and combinations of biomarkers disclosed herein in the manufacture of a kit or biosensor, such as a microarray, for diagnosing or prognosing cancer.

The present invention also provides use of the biomarker panels of the invention (or subset selection thereof) in a method of diagnosis or prognosis of cancer. Such uses are generally in vitro or ex vivo uses. The present invention also provides the use of the biomarker panels of the invention (or subset selection thereof) in the manufacture of a biosensor, such as a microarray, suitable for detection and/or quantification or each of the biomarkers.

When the invention uses one or more biomarkers, the biomarkers may all be measured in a single sample obtained from a patient. Alternatively, multiple samples may be taken from the patient. If multiple samples are available, or if a sample is divided into separate samples, different samples can be used for each gene being measured.

In some embodiments of the invention, the method may comprise providing an expression profile comprising the expression level of each of the genes being measured. A measurement of expression, such as an expression profile, may be provided by quantifying one or more expression products of the genes. The expression products may be proteins or nucleic acids. In some preferred embodiments, the methods comprise quantifying RNA corresponding to the genes being measured. In other preferred embodiments, the methods comprise quantifying proteins corresponding to the genes being measured, for example using immunohistochemical methods.

Thus, in one embodiment of the invention there is provided a method comprising:
(i) providing or obtaining a patient sample;
(ii) determining the gene expression profile of the sample, wherein the gene expression profile is based on the expression the at least two genes being measured;
(iii) optionally correlating the gene expression profile of the sample to a reference; and
(iv) diagnosing or prognosing cancer in the patient.

In some embodiments of the invention, the method comprises contacting the sample with a binding molecule or binding molecules specific for the at least two genes being measured. The binding molecule can be any suitable binding molecule, for example a nucleic acid, an antibody, an antibody fragment, a protein or an aptamer, depending on the method being used.

Measurement of the genes/biomarkers in the sample generally comprises a measurement of the level of expression of the gene. This may be carried out using any suitable means, for example a measurement or analysis of expression products, such as proteins or nucleic acids. Analysis of RNA may be preferred. The RNA may be converted to cDNA prior to analysis. In other embodiments, immunohistochemical analysis, or other methods of quantification of proteins, may be preferred.

Levels of expression may be determined by, for example, quantifying the expression products (such as nucleic acids (e.g. RNA) or proteins) of the biomarkers in the sample (such as a tissue sample). Methods include real-time quantitative PCR, microarray analysis, RNA sequencing, Northern blot analysis and in situ hybridisation. There is also an nCounter Analysis system from NanoString and ‘Integrated Comprehensive Droplet Digital Detection’ (IC 3D) that has been developed for the digital quantification of RNA directly in plasma (K. Zhang, et al., Lab on a Chip, first published online 14 Sep. 2015; DOI: 10.1039/C5LC00650C). In this system the plasma sample containing target RNAs is encapsulated into microdroplets, enzymatically amplified and digitally counted using a novel, high-throughput 3D particle counter.

Methods of real-time qPCR can use stem-loop primers or a poly(A)tailing technique, to reverse transcribe RNA into complementary DNA (cDNA) for the amplification step. Generally using pre-designed assays that target specific RNAs of interest, microarray analysis may comprise the steps of fluorescently labelling the RNAs, hybridization of the labelled RNAs to DNA (or RNA or LNA) probes on a solid-substrate array, washing the array, and scanning the array. RNA enrichment techniques may be particularly useful in methods involving microarrays.

RNA sequencing is another method that can benefit from RNA enrichment, although this is not always necessary. RNA sequencing techniques generally use next generation sequencing methods (also known as high-throughput or massively parallel sequencing). These methods use a sequencing-by-synthesis approach and allow relative quantification and precise identification of RNA sequences. In situ hybridisation techniques can be used on tissue samples, both in vivo and ex vivo.

In some methods of the invention, detection and quantification of cDNA-binding molecule complexes may be used to determine RNA expression. For example, RNA transcripts in a sample may be converted to cDNA by reverse-transcription, after which the sample is contacted with binding molecules specific for the RNAs being quantified, detecting the presence of a of cDNA-specific binding molecule complex, and quantifying the expression of the corresponding gene. There is therefore provided the use of cDNA transcripts corresponding to one or more of the RNAs of interest, or combinations thereof, for use in methods of detecting, diagnosing or prognosis on cancer. In some embodiments of the invention, the method may therefore comprise a step of conversion of the RNAs to cDNA to allow a particular analysis to be undertaken and to achieve RNA quantification.

Methods for detecting the levels of protein expression include any methods known in the art. For example, protein levels can be measured indirectly using DNA or mRNA arrays. Alternatively, protein levels can be measured directly by measuring the level of protein synthesis or measuring protein concentration.

DNA and RNA arrays (microarrays) for use in quantification of the RNAs of interest comprise a series of microscopic spots of DNA or RNA oligonucleotides, each with a unique sequence of nucleotides that are able to bind complementary nucleic acid molecules. In this way the oligonucleotides are used as probes to which only the correct target sequence will hybridise under high-stringency conditions. In the present invention, the target sequence can be the coding DNA sequence or unique section thereof, corresponding to the RNA whose expression is being detected. Most commonly the target sequence is the RNA biomarker of interest itself.

Protein microarrays can also be used to directly detect protein expression. These are similar to DNA and RNA microarrays in that they comprise capture molecules fixed to a solid surface.

Capture molecules include antibodies, proteins, aptamers, nucleic acids, receptors and enzymes, which might be preferable if commercial antibodies are not available for the analyte being detected. Capture molecules for use on the arrays can be externally synthesised, purified and attached to the array. Alternatively, they can be synthesised in-situ and be directly attached to the array. The capture molecules can be synthesised through biosynthesis, cell-free DNA expression or chemical synthesis. In-situ synthesis is possible with the latter two. The appropriate capture molecule will depend on the nature of the target (e.g. mRNA, protein or cDNA).

Once captured on a microarray, detection methods can be any of those known in the art. For example, fluorescence detection can be employed. It is safe, sensitive and can have a high resolution. Other detection methods include other optical methods (for example colorimetric analysis, chemiluminescence, label free Surface Plasmon Resonance analysis, microscopy, reflectance etc.), mass spectrometry, electrochemical methods (for example voltametry and amperometry methods) and radio frequency methods (for example multipolar resonance spectroscopy).

With respect to protein biomarkers, direct measurement of protein expression and identification of the proteins being expressed in a given sample can be done by any one of a number of methods known in the art. For example, 2-dimensional polyacrylamide gel electrophoresis (2D-PAGE) has traditionally been the tool of choice to resolve complex protein mixtures and to detect differences in protein expression patterns between normal and diseased tissue. Differentially expressed proteins observed between normal and tumour samples are separate by 2D-PAGE and detected by protein staining and differential pattern analysis. Alternatively, 2-dimensional difference gel electrophoresis (2D-DIGE) can be used, in which different protein samples are labelled with fluorescent dyes prior to 2D electrophoresis. After the electrophoresis has taken place, the gel is scanned with the excitation wavelength of each dye one after the other. This technique is particularly useful in detecting changes in protein abundance, for example when comparing a sample from a healthy subject and a sample form a diseased subject.

Commonly, proteins subjected to electrophoresis are also further characterised by mass spectrometry methods. Such mass spectrometry methods can include matrix-assisted laser desorption/ionisation time-of-flight (MALDI-TOF).

MALDI-TOF is an ionisation technique that allows the analysis of biomolecules (such as proteins, peptides and sugars), which tend to be fragile and fragment when ionised by more conventional ionisation methods. Ionisation is triggered by a laser beam (for example, a nitrogen laser) and a matrix is used to protect the biomolecule from being destroyed by direct laser beam exposure and to facilitate vaporisation and ionisation. The sample is mixed with the matrix molecule in solution and small amounts of the mixture are deposited on a surface and allowed to dry. The sample and matrix co-crystallise as the solvent evaporates.

Protein microarrays can also be used to directly detect protein expression. These are similar to DNA and mRNA microarrays in that they comprise capture molecules fixed to a solid surface. Capture molecules are most commonly antibodies specific to the proteins being detected, although antigens can be used where antibodies are being detected in serum. Further capture molecules include proteins, aptamers, nucleic acids, receptors and enzymes, which might be preferable if commercial antibodies are not available for the protein being detected. Capture molecules for use on the protein arrays can be externally synthesised, purified and attached to the array. Alternatively, they can be synthesised in-situ and be directly attached to the array. The capture molecules can be synthesised through biosynthesis, cell-free DNA expression or chemical synthesis. In-situ synthesis is possible with the latter two. There is therefore provided a protein microarray comprising capture molecules (such as antibodies) specific for each of the biomarkers being quantified immobilised on a solid support.

Once captured on a microarray, detection methods can be any of those known in the art. For example, fluorescence detection can be employed. It is safe, sensitive and can have a high resolution. Other detection methods include other optical methods (for example colorimetric analysis, chemiluminescence, label free Surface Plasmon Resonance analysis, microscopy, reflectance etc.), mass spectrometry, electrochemical methods (for example voltametry and amperometry methods) and radio frequency methods (for example multipolar resonance spectroscopy).

Additional methods of determine protein concentration include mass spectrometry and/or liquid chromatography, such as LC-MS, UPLC, or a tandem UPLC-MS/MS system.

Methods of the invention involving quantitative analysis, such as quantitative microarray analysis, may be preferred.

Immunohistochemical methods are useful in the present invention for quantification of gene expression. Such methods are known to the person of skill in the art, for example those discussed in Cregger et al., 2006, Arch Pathol Lab Med, 130(7):1026-1030. An example of a suitable technique is paraffin-embedded Q-IHC.

Once the level of expression or concentration has been determined, the level can be compared to a previously measured level of expression or concentration (either in a sample from the same subject but obtained at a different point in time, or in a sample from a different subject, for example a healthy subject, i.e. a control or reference sample) to determine whether the level of expression or concentration is higher or lower in the sample being analysed. Hence, the methods of the invention may further comprise a step of correlating said detection or quantification with a control or reference to determine if cancer is present (or suspected) or not, or to determine the cancer prognosis. Said correlation step may also detect the presence of particular types of cancer and to distinguish these patients from healthy patients, in which no cancer is present. In particular, the invention is particularly useful for predicting cancer metastasis.

Said step of correlation may include comparing the amount (expression or concentration) of the biomarkers with the amount of the corresponding biomarker(s) in a reference sample, for example in a biological sample taken from a healthy patient. Generally, the methods of the invention do not include the steps of determining the amount of the corresponding biomarker in a reference sample, and instead such values will have been previously determined. However, in some embodiments the methods of the invention may include carrying out the method steps from a healthy patient who is used as a control. Alternatively, the method may use reference data obtained from samples from the same patient at a previous point in time. In this way, the effectiveness of any treatment can be assessed and a prognosis for the patient determined.

Internal controls can be also used, for example quantification of one or more different RNAs or proteins not part of the biomarker panel. This may provide useful information regarding the relative amounts of the biomarkers in the sample, allowing the results to be adjusted for any variances according to different populations or changes introduced according to the method of sample collection, processing or storage. Therefore, in some embodiments of the invention, the method may comprise the step of comparing the measured level of expression with one or more housekeeping genes. Suitable housekeeping genes are known to the skilled person.

As would be apparent to a person of skill in the art, any measurements of analyte concentration or expression may need to be normalised to take in account the type of test sample being used and/or and processing of the test sample that has occurred prior to analysis. Data normalisation also assists in identifying biologically relevant results. Invariant RNAs may be used to determine appropriate processing of the sample. Differential expression calculations may also be conducted between different samples to determine statistical significance.

In some embodiments of the invention, the methods comprise determining a ratio of the average expression level of the genes positively correlated with disease score to that of the remaining negatively correlated genes. This ratio is termed the matrix index and is indicative of metastasis and can be used to calculate the hazard ratio, which is indicative of the probability of patient survival.

In general, the methods of the present invention may comprise the steps of:
a) providing or obtaining a biological sample, such as a tissue sample or bodily fluid sample (such as a blood or urine sample);
b) optionally processing the sample, for example to extract the gene expression products (for example RNA or protein) from the sample;
c) quantification of the gene expression products (such as RNA or protein) in the sample.

The methods may further comprise the step of:
d) comparison of the level of gene expression from step c) with a control or reference sample or value.

Alternatively, the method may comprise the step of:
a) determining the average level of gene expression of the genes positively correlated with disease;
b) determining the average level of gene expression of the genes negatively correlated with disease;
c) determining a ratio of expression of the value determined in step (d) and the value determining in step (e); and optionally
d) determining a hazard ratio by associating matrix index with patient survival

The above methods provide a hazard ratio and gives an indication of the prognosis of the diseases (such as the risk of metastasis and/or an indication of the probability of long-term survival of the patient). The average level of gene expression of the genes or proteins may be normalised prior to determining the ration of expression.

A hazard ratio, for example a multivariate hazard ratio, may be determined by any suitable method known to the skilled person. For example, a hazard ratio may be derived from a Cox proportional hazards regression model. Such an analysis allows easier comparison across cancer types and/or datasets using the matrix index.

In embodiments where only one gene that is positively correlated with disease is measured, then no average needs to be determined. Similarly, in embodiments where only one gene that is negatively correlated with disease is measured, then no average needs to be determined. Instead, the expression level of the positively and/or negatively correlated gene can be used to determine the ratio of expression.

The inventors have noted that COL11A1, COMP, FN1, VCAN, CTSB and COL1A1 are positively correlated with disease (i.e. higher expression is correlated with a poorer prognosis), and the remaining genes in the 22 biomarker panel are negatively associated with disease (i.e. a higher expression is correlated with a better prognosis). In other words, an increase in the level of expression of COL11A1, COMP, FN1, VCAN, CTSB and/or COL1A1 is associated with an increased risk of disease or poorer prognosis (e.g. metastasis) and a decrease in the level of the expression of one or more of the remaining genes in the 22 biomarker panel is associated with an increased risk of disease or a poorer prognosis (e.g. metastasis). This is particularly the case when determining the level of gene expression (rather than the level of protein expression).

Accordingly, in some embodiments of the invention, the method requires the expression level of at least one of COL11A1, COMP, FN1, VCAN, CTSB and COL1A1 to be determined, and the expression level of at least one of ANXA6, LGALS3, ANXA1, AB13BP, LAMB1, CTSG, LAMA4, TNXB, AGT, FBLN2, HSPG2, COL6A6, ANXA5, LAMC1, COL15A1 and VWF to be determined, so a ratio of the average level of expression of positively correlated genes with negatively correlated genes can be provided. The level of expression of each of the genes that are measured may be normalised prior to determining the average level of expression and/or determining the ratio of expression.

Where the hazard ratio is greater than 1 (preferably with a confidence internal of at least 95%), a poor prognosis is indicated and the probability of metastasis is increased. Where the hazard ratio is less than 1 (preferably with a confidence interval of at least 95%), a better prognosis is indicated and the probability of metastasis is decreased.

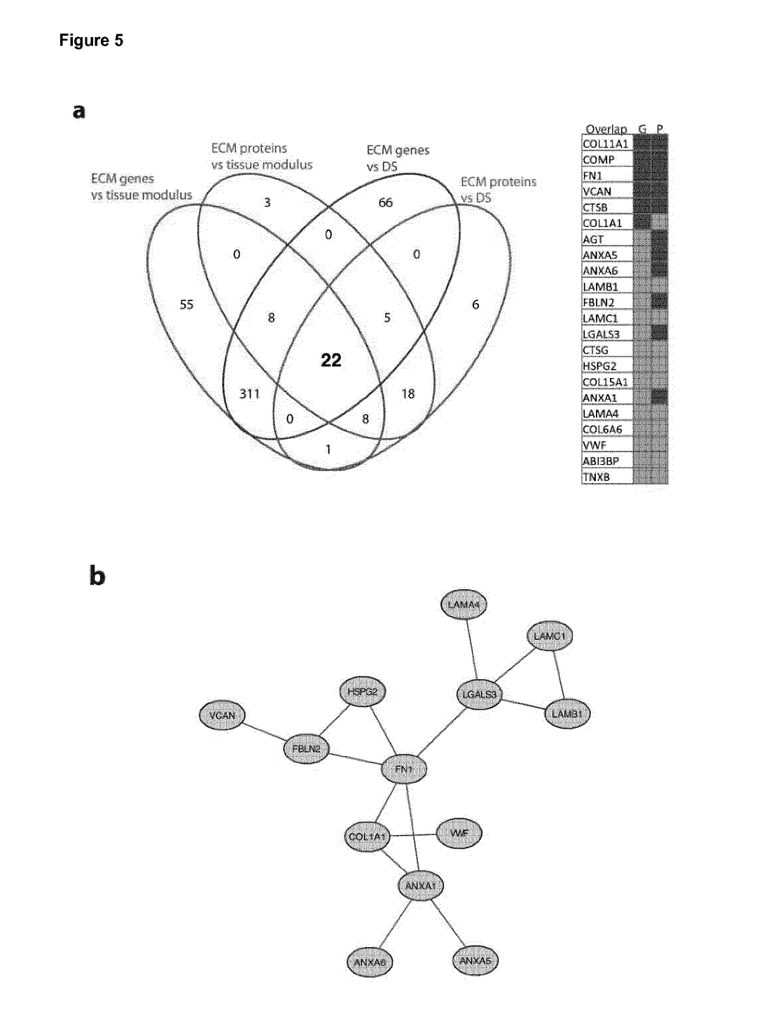

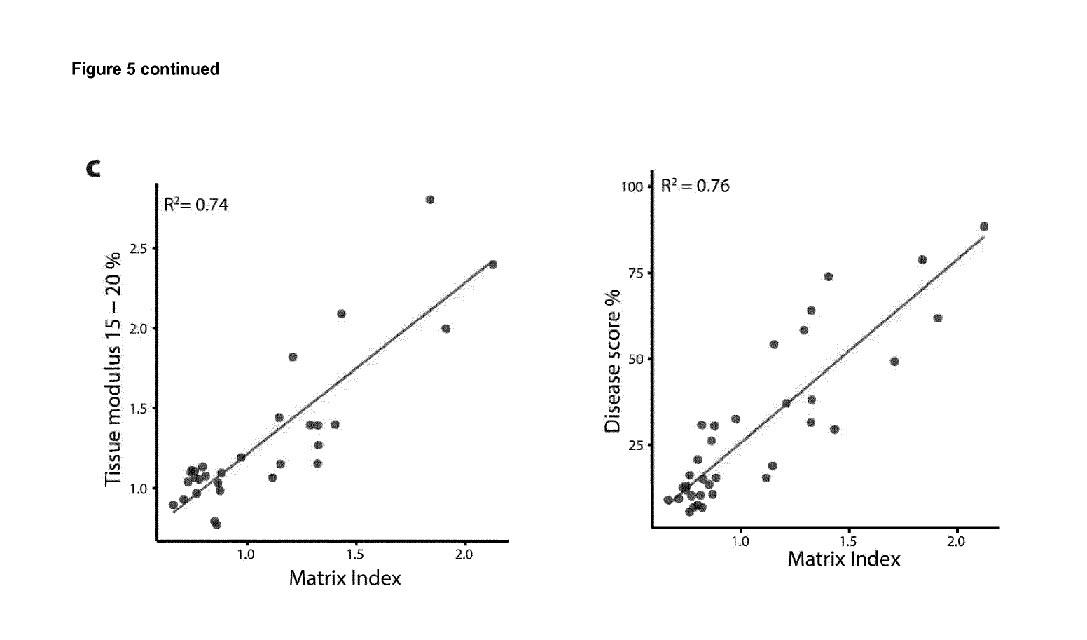

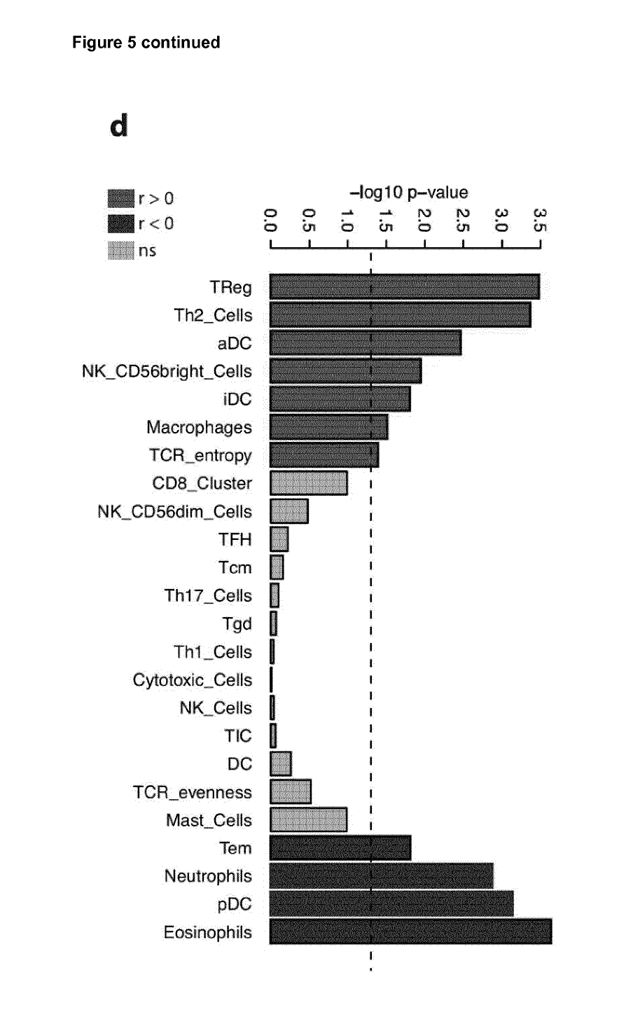

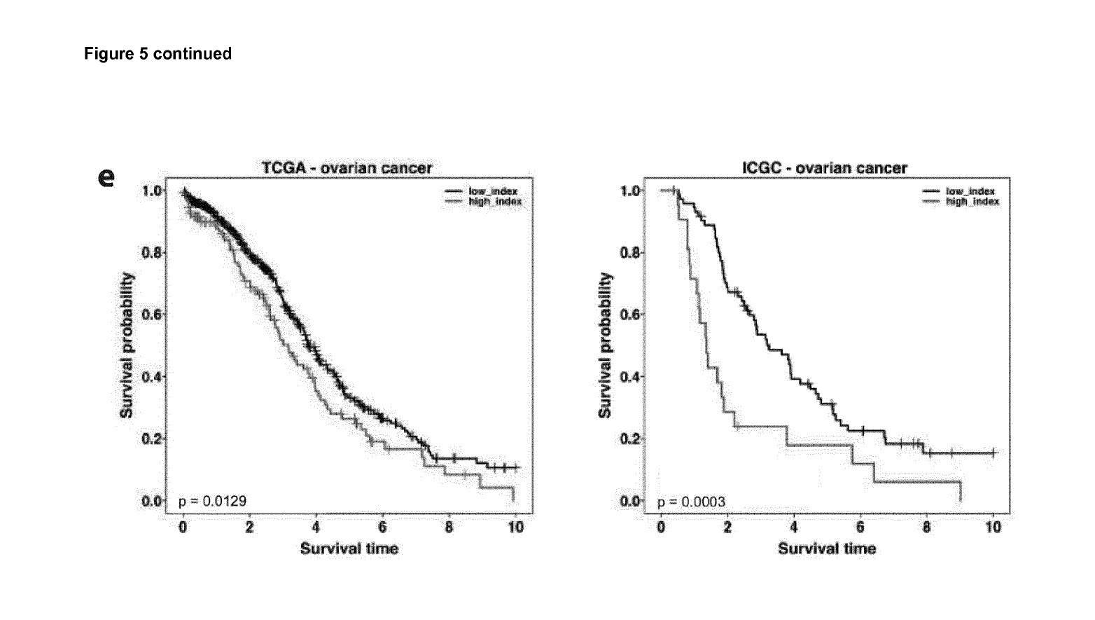

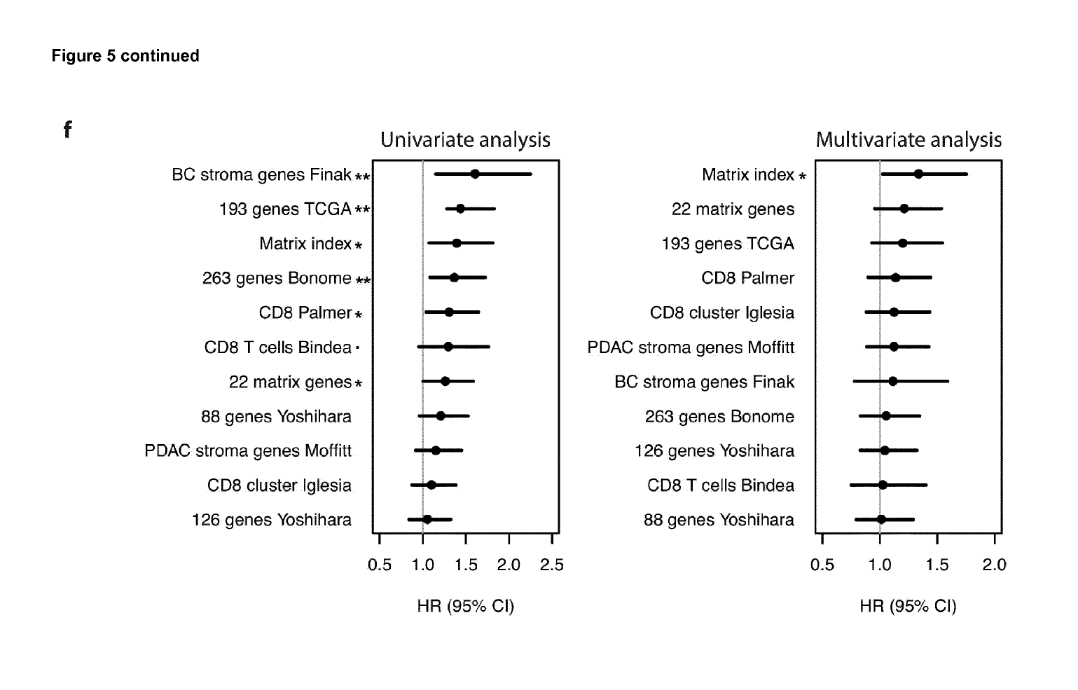

When looking at the level of protein expression in the panel of 22 there are more molecules which are upregulated with disease. These are, COL11A1, COMP, FN1, VCAN, CTSB, AGT, ANXA5, ANXA6, FBLN2, LGALS3 and ANXA1 and the remaining proteins in the 22 panel are negatively correlated with disease (as shown in FIG. 5A). Therefore the matrix index at protein level should be calculated with this in mind.

At gene level the matrix index allows cancer prognosis to be determined. The higher the matrix index, the worse the patient's prognosis. Matrix index may be defined simply as the level of expression (or average level of expression) of the positively correlated genes divided by the level of expression (or average level of expression) of the negatively correlated genes.

In one embodiment of the invention, the method comprises determining a ratio of expression of genes positively correlated with disease score to expression of genes negatively correlated with disease score, wherein genes positively correlated are COL11A1, COMP, FN1, VCAN, CTSB and COL1A1 and genes negatively correlated with disease score are ANXA6, LGALS3, ANXA1, AB13BP, LAMB1, CTSG, LAMA4, TNXB, AGT, FBLN2, HSPG2, COL6A6, ANXA5, LAMC1, COL15A1 and VWF. The method may comprise:
(i) determining an average level of gene expression for the genes positively correlated with disease score whose expression level is quantified;
(ii) determining an average level of gene expression for the genes negatively correlated with disease score whose expression level is quantified; and
(iii) providing a matrix index, wherein the matrix index is the average level of expression of the positively correlated genes determined in step (i) divided by the average level of expression of the negatively correlated genes determined in step (ii). Of course, if only one gene is used in either of the positively or negatively correlated gene groups, then no average needs to be calculated and the “average” in this context is the level of expression of that one gene whose expression level is quantified. In some embodiments, the method may further comprise calculating a hazard ratio from the matrix index, wherein the hazard ratio is indicative of the probability of patient survival. Furthermore, the methods may comprise normalisation of the gene expression levels, and/or comparison of the gene expression levels to control or reference genes, as described herein.

Certain aspect of methods of the invention may be carried out by a computer. The present invention therefore provides a computer programmed to carry out the methods of the invention, for example to determine average levels of expression of genes in the gene panel, determine a ratio of positively to negatively correlated genes, determine a matrix index and/or determine a hazard ratio as described herein. The computer may be further programmed to generate a report providing the results of the calculations, for example the matrix index and/or the hazard ratio.

In some embodiments of the invention, the step of quantification of gene expression may comprise the following steps:
a) contacting the sample or extracted RNA or protein with a binding partner that specifically binds to the RNA(s) or protein(s) of interest
b) quantifying the amount of RNA-binding partners or protein-binding partners to determine the amount of the RNA(s) or protein(s) present in the original sample.

The present invention therefore provides a reaction mixture, comprising either the RNAs or proteins of interest, or a biological sample (such as a tissue sample) containing the RNAs or proteins of interest, wherein the RNAs or proteins of interest are bound to a binding partner specific to the RNA or protein. The binding partner may be, for example, an oligonucleotide that hybridises to the RNA, or an antibody or antigen binding fragment thereof that specifically binds to the protein.

Alternatively, the reaction mixture may comprise cDNA molecules corresponding to the RNAs of interest, and it is the cDNAs that are bound to a specific binding partner. The RNAs of interest correlate to the genes of the biomarkers being analysed.

The method of the invention can be carried out using a binding molecules or reagents specific for the expression products or cDNAs being detected. Binding molecules and reagents are those molecules that have an affinity for the target such that they can form binding molecule/reagent-biomarker complexes that can be detected using any method known in the art. The binding molecule of the invention can be an antibody, an antibody fragment, a nucleic acid, an oligonucleotide, a protein or an aptamer or molecularly imprinted polymeric structure, depending on the nature of the target (for example RNA or, in some embodiments, cDNA or protein). Methods of the invention may comprise contacting the biological sample with an appropriate binding molecule or molecules. Said binding molecules may form part of a kit of the invention, in particular they may form part of the biosensors of in the present invention.

Antibodies can include both monoclonal and polyclonal antibodies and can be produced by any means known in the art. Techniques for producing monoclonal and polyclonal antibodies which bind to a particular protein are now well developed in the art. They are discussed in standard immunology textbooks, for example in Roitt et al., Immunology, second edition (1989), Churchill Livingstone, London. Polyclonal antibodies can be raised by stimulating their production in a suitable animal host (e.g. a mouse, rat, guinea pig, rabbit, sheep, chicken, goat or monkey) when the antigen is injected into the animal. If necessary, an adjuvant may be administered together with the antigen. The antibodies can then be purified by virtue of their binding to antigen or as described further below. Monoclonal antibodies can be produced from hybridomas. These can be formed by fusing myeloma cells and B-lymphocyte cells which produce the desired antibody in order to form an immortal cell line. This is the well known Kohler & Milstein technique (Kohler & Milstein (1975) Nature, 256:52-55). The antibodies may be human or humanised, or may be from other species.

The present invention includes antibody derivatives which are capable of binding to antigen. Thus, the present invention includes antibody fragments and synthetic constructs. Examples of antibody fragments and synthetic constructs are given in Dougall et al. (1994) Trends Biotechnol, 12:372-379.

Antibody fragments or derivatives, such as Fab, F(ab′)2 or Fv may be used, as may single-chain antibodies (scAb) such as described by Huston et al. (993) Int Rev Immunol, 10:195-217, domain antibodies (dAbs), for example a single domain antibody, or antibody-like single domain antigen-binding receptors. In addition, antibody fragments and immunoglobulin-like molecules, peptidomimetics or non-peptide mimetics can be designed to mimic the binding activity of antibodies. Fv fragments can be modified to produce a synthetic construct known as a single chain Fv (scFv) molecule. This includes a peptide linker covalently joining VH and VL regions which contribute to the stability of the molecule. The present invention therefore also extends to single chain antibodies or scAbs.

Other synthetic constructs include CDR peptides. These are synthetic peptides comprising antigen binding determinants. These molecules are usually conformationally restricted organic rings which mimic the structure of a CDR loop and which include antigen-interactive side chains. Synthetic constructs also include chimeric molecules. Thus, for example, humanised (or primatised) antibodies or derivatives thereof are within the scope of the present invention. An example of a humanised antibody is an antibody having human framework regions, but rodent hypervariable regions. Synthetic constructs also include molecules comprising a covalently linked moiety which provides the molecule with some desirable property in addition to antigen binding. For example the moiety may be a label (e.g. a detectable label, such as a fluorescent or radioactive label) or a pharmaceutically active agent.

In those embodiments of the invention in which the binding molecule is an antibody or antibody fragment, the method of the invention can be performed using any immunological technique known in the art. For example, ELISA, radio immunoassays, bead-based, or similar techniques may be utilised. In general, an appropriate autoantibody is immobilised on a solid surface and the sample to be tested is brought into contact with the autoantibody. If the cancer biomarker recognised by the autoantibody is present in the sample, an antibody-marker complex is formed. The complex can then be directed or quantitatively measured using, for example, a labelled secondary antibody which specifically recognises an epitope of the biomarker. The secondary antibody may be labelled with biochemical markers such as, for example, horseradish peroxidase (HRP) or alkaline phosphatase (AP), and detection of the complex can be achieved by the addition of a substrate for the enzyme which generates a colorimetric, chemiluminescent or fluorescent product. Alternatively, the presence of the complex may be determined by addition of a protein labelled with a detectable label, for example an appropriate enzyme. In this case, the amount of enzymatic activity measured is inversely proportional to the quantity of complex formed and a negative control is needed as a reference to determining the presence of antigen in the sample. Another method for detecting the complex may utilise antibodies or antigens that have been labelled with radioisotopes followed by a measure of radioactivity. Examples of radioactive labels for antigens include ³H, ¹⁴C and ¹²⁵I.

Aptamers are oligonucleotides or peptide molecules that bind a specific target molecule. Oligonucleotide aptamers include DNA aptamers and RNA aptamers. Aptamers can be created by an in vitro selection process from pools of random sequence oligonucleotides or peptides. Aptamers can be optionally combined with ribozymes to self-cleave in the presence of their target molecule.

Aptamers can be made by any process known in the art. For example, a process through which aptamers may be identified is systematic evolution of ligands by exponential enrichment (SELEX). This involves repetitively reducing the complexity of a library of molecules by partitioning on the basis of selective binding to the target molecule, followed by re-amplification. A library of potential aptamers is incubated with the target biomarker before the unbound members are partitioned from the bound members. The bound members are recovered and amplified (for example, by polymerase chain reaction) in order to produce a library of reduced complexity (an enriched pool). The enriched pool is used to initiate a second cycle of SELEX. The binding of subsequent enriched pools to the target biomarker is monitored cycle by cycle. An enriched pool is cloned once it is judged that the proportion of binding molecules has risen to an adequate level. The binding molecules are then analysed individually. SELEX is reviewed in Fitzwater & Polisky (1996) Methods Enzymol, 267:275-301.

Thus, in one embodiment of the invention, there is provided a method of analysing a biological sample from a patient, comprising contacting the sample with reagents or binding molecules specific for the biomarker(s) being quantified, and measuring the abundance of biomarker-reagent or biomarker-binding molecule complexes, and correlating the abundance of biomarker-reagent or biomarker-binding molecule complexes with the concentration of the relevant biomarker in the biological sample. For example, in one embodiment of the invention, the method comprises the steps of:
a) contacting a biological sample with reagents or binding molecules specific for one or more of the genes in a biomarker panel of the invention;
b) quantifying the abundance of biomarker-reagent or biomarker-binding molecule complexes for at least two genes in a biomarker panel of the invention; and
c) correlating the abundance of biomarker-reagent or biomarker-binding molecule complexes with the concentration or expression of at least two genes in a biomarker panel of the invention in the biological sample.

The method may further comprise the step of d) comparing the concentration or expression of the biomarkers in step c) with a reference to diagnose or prognose cancer. The patient can then be treated accordingly. Alternatively, a ratio between the genes positively correlated with disease to the genes negatively associated with disease may be determined. As discussed elsewhere, suitable reagents or binding molecules may include an antibody or antibody fragment, an enzyme, a nucleic acid, an organelle, a cell, a biological tissue, imprinted molecule or a small molecule. Such methods may be carried out using kits or biosensors of the invention.

### Other Methods of the Invention

The present invention also provides methods of treatment of cancer in a patient. A sample from the patient may have undergone a method of diagnosis or prognosis of the invention to determine the patient's suitability for treatment. In some embodiments, the methods of treatment include the steps of diagnosis or prognosis according to a method of the invention.

In some embodiments, the methods comprise only recommending the patient for, or assigning a treatment to, the patient. In other embodiments, the methods include the steps of treatment administration.

In one embodiment of the invention, the method comprises:
(i) providing or obtaining a sample from a patient;
(ii) measuring the level of expression of at least two genes from the biomarker panels of the invention in the patient sample;
(iii) determining the presence or absence of cancer based on the measurement in step (ii); and
(iv) administering a cancer therapy or initiating a therapeutic regimen for cancer if cancer is diagnosed or suspected

In another embodiment of the invention, the method comprises:
(i) providing or obtaining a sample from a patient;
(ii) optionally enriching the sample for protein or RNA and/or extracting protein or RNA from the sample;
(iii) diagnosing or prognosing cancer according to a method of diagnosis or prognosis of the invention; and
(iv) selecting a treatment regimen for the patient according to the presence or absence cancer as determined in step (iii).

In another embodiment of the invention, there is provided a method of predicting a patient's responsiveness to a cancer treatment, comprising
(i) providing or obtaining a sample from a patient;
(ii) optionally enriching the sample for protein or RNA and/or extracting protein or RNA from the sample;
(iii) diagnosing or prognosing cancer according to a method of diagnosis or prognosis of the invention;
(iv) predicting a patient's responsiveness to a cancer treatment according to the presence or absence of cancer as determined in step (iii).

The treatment being administered will depend on the cancer that is being analysed. The treatment can be chemotherapy and/or radiotherapy.

Typical chemotherapeutic agents include alkylating agents (for example nitrogen mustards (such as mechlorethamine, cyclophosphamide, melphalan, chlorambucil, ifosfamide and busulfan), nitrosoureas (such as N-Nitroso-N-methylurea (MNU), carmustine (BCNU), lomustine (CCNU) and semustine (MeCCNU), fotemustine and streptozotocin), tetrazines (such as dacarbazine, mitozolomide and temozolomide), aziridines (such as thiotepa, mytomycin and diaziquone), cisplatins and derivatives thereof (such as carboplatin and oxaliplatin), and non-classical alkylating agents (such as procarbazine and hexamethylmelamine)), antimetabolites (for example anti-folates (such as methotrexate and pemetrexed), fluoropyrimidines (such as fluorouracil and capecitabine), deoxynucleoside analogues (such as cytarabine, gemcitabine, decitabine, Vidaza, fludarabine, nelarabine, cladribine, clofarabine and pentostatin) and thiopurines (such as thioguanine and mercaptopurine)), anti-microtubule agents (for example Vinca alkaloids (such as vincristine, vinblastine, vinorelbine, vindesine, and vinflunine) and taxanes (such as paclitaxel and docetaxel)), platins (such as cisplatin and carboplatin), topoisomerase inhibitors (for example irinotecan, topotecan, camptothecin, etoposide, doxorubicin, mitoxantrone, teniposide, novobiocin, merbarone, and aclarubicin), and cytotoxic antibiotics (for example anthracyclines (such as doxorubicin, daunorubicin apirubicin, idarubicin, pirarubicin, aclarubicin, mitoxantrone), bleomycins, mitomycin C, mitoxantrone, and actinomycin), and combinations thereof.

Of particular relevance to the present invention (i.e. in those embodiments relating in particular to epithelial cancers, such as ovarian cancer) are the platins and taxanes (such as carboplatin in combination with paclitaxel (although cisplatin can be used instead of carboplatin, and/or docetaxel can be used instead of paclitaxel). Other chemotherapeutic agents of particular relevance to the present invention include altretamine, capecitabine, cyclophosphamide, etoposide (VP-16), gemcitabine, irinotecan, doxorubicin, melphalan, pemetrexed, topotecan, and vinorelbine, TGF-beta inhibitors may also be used.

The treatment regimen may comprise surgery, for example resection of a tumour. In particular, resection may be recommended in metastasis has been predicted or is suspected.

### Biological Samples

In the present invention, the biological sample may be a surgical sample. The sample can be a liquid biopsy sample, for example blood, plasma, serum, urine, seminal fluid, stool, sputum, pleural fluid, ascetic fluid, synovial fluid, cerebrospinal fluid, lymph, nipple fluid, cyst fluid or bronchial lavage. In some embodiments, the sample is a cytological sample or smear or a fluid containing cellular material, such as cervical smear, nasal brushing, or esophageal sampling by a sponge (cytosponge), endoscopic/gastroscopic/colonoscopic biopsy or brushing, cervical mucus or brushing. In preferred embodiments, the sample is a tissue sample (i.e. a biopsy), in particular a tumour sample, or a blood or urine sample.

The invention may include a step of obtaining or providing the biological sample, or alternatively the sample may have already been obtained from a patient, for example in ex vivo methods.

Biological samples obtained from a patient can be stored until needed. Suitable storage methods include freezing within two hours of collection. Maintenance at −80° C. can be used for long-term storage.

The sample may be processed prior to determining the level of expression of the biomarkers. The sample may be subject to enrichment (for example to increase the concentration of the biomarkers being quantified), centrifugation or dilution. Expression products of the genes (such as protein or nucleic acids, but in particular RNA) may be extracted from the sample prior to analysis.

In some embodiments of the invention, the biological sample may be enriched for gene expression products prior to detection and quantification (i.e. measurement). The step of enrichment can be any suitable pre-processing method step to increase the concentration of gene expression products in the sample. For example, the step of enrichment may comprise centrifugation and filtration to remove cells or unwanted analytes from the sample. For RNA, methods of the invention may include a step of amplification to increase the amount of RNA that is detected and quantified. Methods of amplification include PCR amplification. Such methods may be used to enrich the sample for any biomarkers of interest.

Generally speaking, the gene expression products will need to be extracted from the biological sample. This can be achieved by a number of suitable methods. For example, extraction may involve separating the gene expression products from the biological sample. Methods include chemical extraction (comprising the use of, for example, guanidium thiocyante) and solid-phase extraction (for example on silica columns). Preferred methods include chromatographic methods (for example spin column chromatography), in particular chromatographic methods comprising the use of a silica column. Chromatographic methods comprise lysing cells (if required), addition of a binding solution, centrifugation in a spin column to force the binding solution through a silica gel membrane, optional washing to remove further impurities, and elution of the nucleic acid.

Commercial kits are available for such methods, for example Norgen's urine microRNA purification kit (other kits available, for example from Qiagen or Exigon).

If gene expression products such as RNA are extracted from a sample, the extracted solution may require enrichment to increase the relative abundance of RNA in the sample.

In one embodiment of the invention, the method the sample is processed prior to analysis, wherein processing of the sample comprises:
(i) removal of cells and/or debris from the sample;
(ii) optional purification of the sample to obtained a purified sample comprising expression products (for example protein or nucleic acid molecules) corresponding to the genes being measured; and/or
(iii) extraction or isolation expression products (for example protein or nucleic acid molecules) corresponding to the genes being measured.

The methods of the invention may be carried out on one test sample from a patient. Alternatively, a plurality of test samples may be taken from a patient, for example 2, 3, 4 or 5 samples. Each sample may be subjected to a single assay to quantify one of the biomarker panel members, or alternatively a sample may be tested for a plurality of or all of the biomarkers being quantified.

In one embodiment, there is provided a method comprising:
a) measuring at least two genes of the biomarker panels of the invention in a biological sample obtained from a patient that has previously received therapy for cancer;
b) comparing the measurement determined in step a) with a previously determined level of expression of the same biomarker or biomarkers; and
c) maintaining, changing or withdrawing the therapy for cancer.

The method may comprise a prior step of administering the therapy for cancer to the patient. In another embodiment, the method may also comprise a pre-step of measuring one or more genes of the biomarker panels of the invention in a biological sample obtained from the same patient prior to administration of the therapy. In step c), the therapy for cancer may be maintained if an appropriate adjustment in the level(s) of expression of the biomarker or biomarkers is determined. If the levels of expression are unchanged or have worsened, this may be indicative of a worsening of the patient's condition, and hence an alternative therapy for cancer. In this way, drug candidates useful in the treatment of cancer can be screened.

In another embodiment of the invention, there is provided a method identifying a drug useful for the treatment of cancer, comprising:
(a) measuring at least two genes of the biomarker panels of the invention in a biological sample obtained from a patient;
(b) administering a candidate drug to the patient;
(c) measuring at least two genes of the biomarker panels of the invention in a biological sample obtained from the same patient at a point in time after administration of the candidate drug; and
(d) comparing the value determined in step (a) with the value determined in step (c), to determine the suitability of the drug candidate as a treatment for cancer.

### Cancers

The inventors have found that the biomarkers and biomarker panels are useful in the diagnosis in a range of cancers, since they have found the tumour microenvironment, in particular the expression profile of the tumour microenvironment, is similar in a range of cancers.

In preferred embodiments, the cancer is an epithelial cancer or a mesenchymal cancer.

In one embodiment, the cancer is an epithelial cancer.

In some embodiments, the cancer is selected from the group consisting of breast cancer, cervical cancer, mesothelioma, ovarian cancer, liver cancer, lung cancer, oesophageal cancer, sarcoma, colon cancer, head and neck cancer, pancreatic cancer, rectal cancer, thyroid cancer and kidney cancer.

In some embodiments of the invention, the cancer is selected from the group consisting of acute lymphoblastic leukemia, acute or chronic lymphocyctic or granulocytic tumor, acute myeloid leukemia, acute promyelocytic leukemia, adenocarcinoma, adenoma, adrenal cancer, basal cell carcinoma, bone cancer, brain cancer, breast cancer, bronchi cancer, cervical dysplasia, chronic myelogenous leukemia, colon cancer, epidermoid carcinoma, Ewing's sarcoma, gallbladder cancer, gallstone tumor, giant cell tumor, glioblastoma multiforma, hairy-cell tumor, head cancer, hyperplasia, hyperplastic corneal nerve tumor, in situ carcinoma, intestinal ganglioneuroma, islet cell tumor, Kaposi's sarcoma, kidney cancer, larynx cancer, leiomyomater tumor, liver cancer, lung cancer, lymphomas, malignant carcinoid, malignant hypercalcemia, malignant melanomas, marfanoid habitus tumor, medullary carcinoma, metastatic skin carcinoma, mucosal neuromas, mycosis fungoide, myelodysplastic syndrome, myeloma, neck cancer, neural tissue cancer, neuroblastoma, osteogenic sarcoma, osteosarcoma, ovarian cancer, pancreas cancer, parathyroid cancer, pheochromocytoma, polycythemia vera, primary brain tumor, prostate cancer, rectum cancer, renal cell tumor, retinoblastoma, rhabdomyosarcoma, seminoma, skin cancer, small-cell lung tumor, soft tissue sarcoma, squamous cell carcinoma, stomach cancer, thyroid cancer, topical skin lesion, veticulum cell sarcoma, and Wilm's tumor.

In some embodiments, the cancer is selected from the group consisting of triple negative breast cancer, mesothelioma, ovarian cancer, liver hepatocellular carcinoma, lung adenocarcinoma, oesophageal carcinoma, sarcoma, breast invasive carcinoma, colon adenocarcinoma, head and neck squamous cell carcinoma, pancreatic adenocarcinoma and kidney renal clear cell carcinoma.

In some embodiments, the cancer is selected from the group consisting of breast cancer, cervical squamous cell carcinoma, colon adenocarcinoma, rectum adenocarcinoma, oesophageal carcinoma, head-neck squamous cell carcinoma, kidney renal clear cell carcinoma, kidney renal papillary cell carcinoma, liver hepatocellular carcinoma, low grade glioma, lung adenocarcinoma, mesothelioma, ovarian cancer, pancreatic adenocarcinoma, pancreatic cancer endocrine neoplasms, sarcoma, thyroid cancer and triple-negative breast cancer

In some embodiments, the cancer is uveal melanoma, triple negative breast cancer, skin cutaneous melanoma, sarcoma, pancreatic adenocarcinoma, ovarian cancer, mesothelioma, lung squamous cell carcinoma, lung adenocarcinoma, liver hepatocellular carcinoma, kidney papillary cell carcinoma, kidney clear cell carcinoma, head and neck squamous cell carcinoma, glioblastoma multiforme, esophageal carcinoma, diffuse large B-cell lymphoma, colon and rectum adenocarcinoma, colon adenocarcinoma, or breast invasive carcinoma.

In a more preferred embodiment, the cancer is epithelial ovarian cancer, in particular serous ovarian cancer, including high-grade serous ovarian cancer.

In some embodiments, the cancer is selected from the group consisting of glioblastoma, melanoma and lymphoma. In such embodiments, the matrix score may be negatively correlated with disease score (i.e. a higher matrix index is indicative of a better prognosis).

In embodiments where, for example, the 6 gene panel is used (COL11A1, ANXA6, LAMC1, CTSB, LAMA4 and HSPG2), the panel may be of particular relevance to breast cancer, cervical cancer, oesophageal cancer, head and neck cancer, kidney cancer, liver cancer, lung cancer, mesothelioma, ovarian cancer, pancreatic cancer, sarcoma or thyroid cancer, although it may also be applicable to other cancers. For example, the panel may be of particular relevance to breast cancer, cervical squamous cell carcinoma, oesophageal carcinoma, head-neck squamous cell carcinoma, kidney renal clear cell carcinoma, liver hepatocellular carcinoma, lung adenocarcinoma, mesothelioma, ovarian cancer, pancreatic adenocarcinoma, pancreatic cancer endocrine neoplasms, sarcoma, thyroid cancer or triple-negative breast cancer. In such embodiments, the matrix index may be positively correlated with a poorer outcome. The panel may also be of particular relevance to glioblastoma, lung cancer, stomach cancer or uveal melanoma (for example glioblastoma multiforme, lung squamous cell carcinoma, stomach adenocarcinoma or uveal melanoma), wherein the matrix index may be negatively correlated with a poorer outcome.

In embodiments where, for example, the 22 gene panel is used (or subsets thereof), the panel may be of particular relevance to breast cancer, cervical cancer, colon cancer, head and neck cancer, kidney cancer, liver cancer, lung cancer, mesothelioma, ovarian cancer or sarcoma, although it may also be applicable to other cancers. For example, the panel may be of particular relevance to breast cancer, cervical squamous cell carcinoma, colon adenocarcinoma, head-neck squamous cell carcinoma, kidney renal clear cell carcinoma, kidney renal papillary cell carcinoma, liver hepatocellular carcinoma, lung adenocarcinoma, mesothelioma, ovarian cancer or triple-negative breast cancer. In such embodiments, the matrix index may be positively correlated with a poorer outcome. The panel may also be of particular relevance to glioblastoma, lung cancer, skin cancer or uveal melanoma (for example glioblastoma multiforme, lung squamous cell carcinoma, skin cutaneous melanoma or uveal melanoma), wherein the matrix index may be negatively correlated with a poorer outcome.

### Kits and Biosensors

The present invention also relates to a kit for diagnosis or prognosis cancer, comprising means for measuring at least two genes selected from the group consisting of COL11A1, CTS, ANXA6, LGALS3, ANXA1, AB13BP, COMP, COL1A1 , LAMB1, CTSG, LAMA4, TNXB, FN1, AGT, FBLN2, HSPG2, COL6A6, VCAN, ANXA5, LAMC1, COL15A1 and VWF. Other biomarker panels and sub-selections of genes may be used, as discussed above. The kit may comprise instructions for use.

In one embodiment, the kit of parts of the invention may comprise biosensor. A biosensor incorporates a biological sensing element and provides information on a biological sample, for example the presence (or absence) or concentration of an analyte. Specifically, they combine a biorecognition component (a bioreceptor) with a physiochemical detector for detection and/or quantification of an analyte (such as an RNA, a cDNA or a protein).

The bioreceptor specifically interacts with or binds to the analyte of interest and may be, for example, an antibody or antibody fragment, an enzyme, a nucleic acid, an organelle, a cell, a biological tissue, imprinted molecule or a small molecule. The bioreceptor may be immobilised on a support, for example a metal, glass or polymer support, or a 3-dimensional lattice support, such as a hydrogel support.

Biosensors are often classified according to the type of biotransducer present. For example, the biosensor may be an electrochemical (such as a potentiometric), electronic, piezoelectric, gravimetric, pyroelectric biosensor or ion channel switch biosensor. The transducer translates the interaction between the analyte of interest and the bioreceptor into a quantifiable signal such that the amount of analyte present can be determined accurately. Optical biosensors may rely on the surface plasmon resonance resulting from the interaction between the bioreceptor and the analyte of interest. The SPR can hence be used to quantify the amount of analyte in a test sample. Other types of biosensor include evanescent wave biosensors, nanobiosensors and biological biosensors (for example enzymatic, nucleic acid (such as DNA), antibody, epigenetic, organelle, cell, tissue or microbial biosensors).

The invention also provides microarrays (RNA, DNA or protein) comprising capture molecules (such as RNA or DNA oligonucleotides) specific for each of the biomarkers or biomarker panels being quantified, wherein the capture molecules are immobilised on a solid support. The microarrays are useful in the methods of the invention.

In particular, the present invention provides a combination of binding molecules, wherein each binding molecule specifically binds a different target analyte.

The binding molecules may be present on a solid substrate, such an array (for example an RNA microarray, in which case the binding molecules are RNAs that hybridise to the target miRNA). The binding molecules may all be present on the same solid substrate. Alternatively, the binding molecules may be present on different substrates. In some embodiments of the invention, the binding molecules are present in solution.

These kits may further comprise additional components, such as a buffer solution. Other components may include a labelling molecule for the detection of the bound miRNA and so the necessary reagents (i.e. enzyme, buffer, etc) to perform the labelling; binding buffer; washing solution to remove all the unbound or non-specifically bound miRNAs. Hybridisation will be dependent on the size of the putative binder, and the method use may be to be determined experimentally, as is standard in the art. As an example, hybridisation can be performed at ˜20° C. below the melting temperature (Tm), over-night. (Hybridisation buffer: 50% deionised formamide, 0.3 M NaCl, 20 mM Tris-HCl, pH 8.0, 5 mM EDTA, 10 mM phosphate buffer, pH 8.0, 10% dextran sulfate, 1× Denhardt's solution, and 0.5 mg/mL yeast tRNA). Washes can be performed at 4-6° C. higher than hybridization temperature with 50% Formamide/2×SSC (20× Standard Saline Citrate (SSC), pH 7.5: 3 M NaCl, 0.3 M sodium citrate, the pH is adjusted to 7.5 with 1 M HCl). A second wash can be performed with 1×PBS/0.1% Tween 20.

Binding or hybridisation of the binding molecules to the target analyte may occur under standard or experimentally determined conditions. The skilled person would appreciate what stringent conditions are required, depending on the biomarkers being measured. The stringent conditions may include a hybridisation buffer that is be high in salt concentration, and a temperature of hybridisation high enough to reduce non-specific binding.

As used herein, “stringent conditions for hybridization” are known to those skilled in the art and can be found in Current Protocols in Molecular Biology, John Wiley & Sons, N.Y., 6.3.1-6.3.6, 1991. Stringent conditions may be defined as equivalent to hybridization in 6× sodium chloride/sodium citrate (SSC) at 45° C., followed by a wash in 0.2×SSC, 0.1% SDS at 65° C. Alternatively, stringent conditions may be defined as equivalent to hybridization in 50% v/v formamide, 10% w/v Dextran sulphate, 2×SSC at 37° C., followed by a wash in 50% formamide/2×SSC at 42° C.

In one embodiment of the invention, the kit is able to simultaneously measure both miRNA biomarkers and protein biomarkers.

The present invention also provides a microarray, comprising specific binding molecules that hybridize to an expression product from at least two genes of the biomarker panels of the invention. The microarray can be a DNA or RNA microarray. The microarray may comprise a sample from a patient. In some embodiments, the specific binding molecules are oligonucleotides. When in use, the expression products may be hybridized to the corresponding specific binding molecules.

Preferred features for the second and subsequent aspects are as provided for the first aspect, mutatis mutandis.

The invention will now be described with reference to a number of Examples, in which reference is made to a number of figures, as follows:

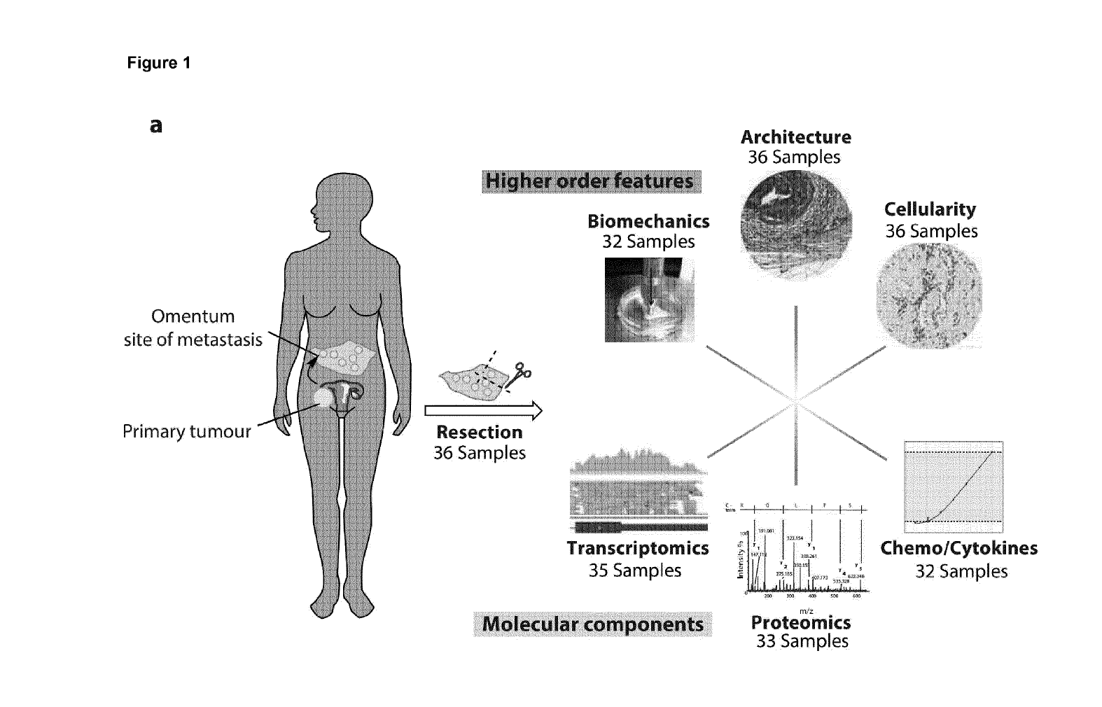

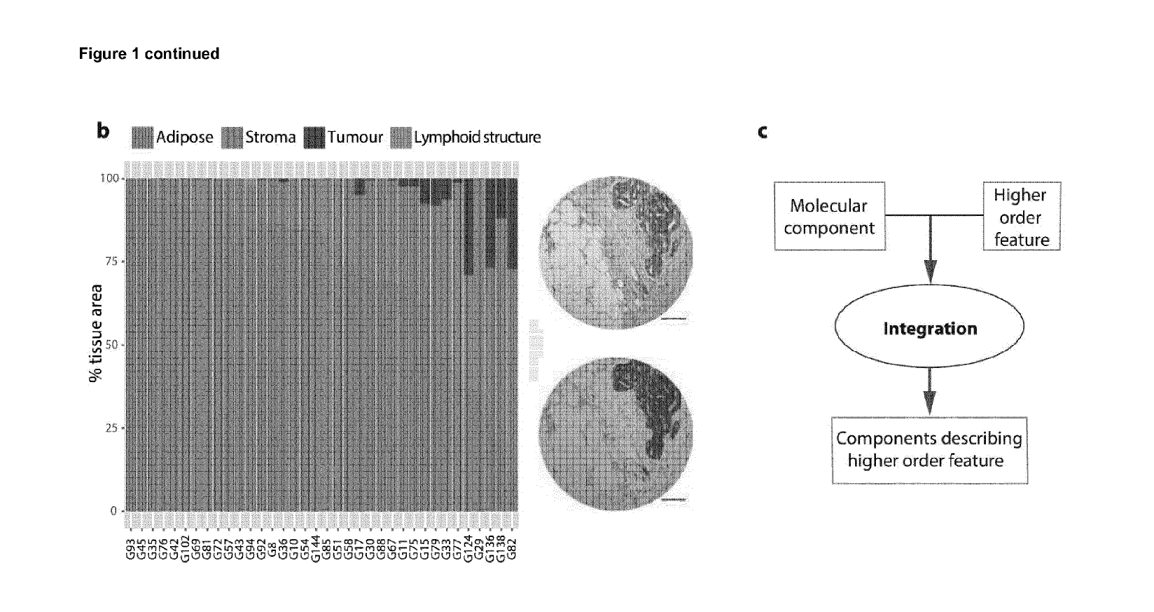

FIG. 1. Study Design and Sample Description

a) Overview of the samples and the analyses conducted on the same tissue specimen.

b) Digital analysis of architecture of each sample based on percentage of malignant cell area (−tumor), stroma, and adipocyte area. The combined percentage area occupied by tumor and stroma was used to determine the ‘disease score’ of each sample. Scale-bars correspond to 100 μm. c) Schematic of the PLS regression method used to define higher-order features of the tumor microenvironment from molecular components.

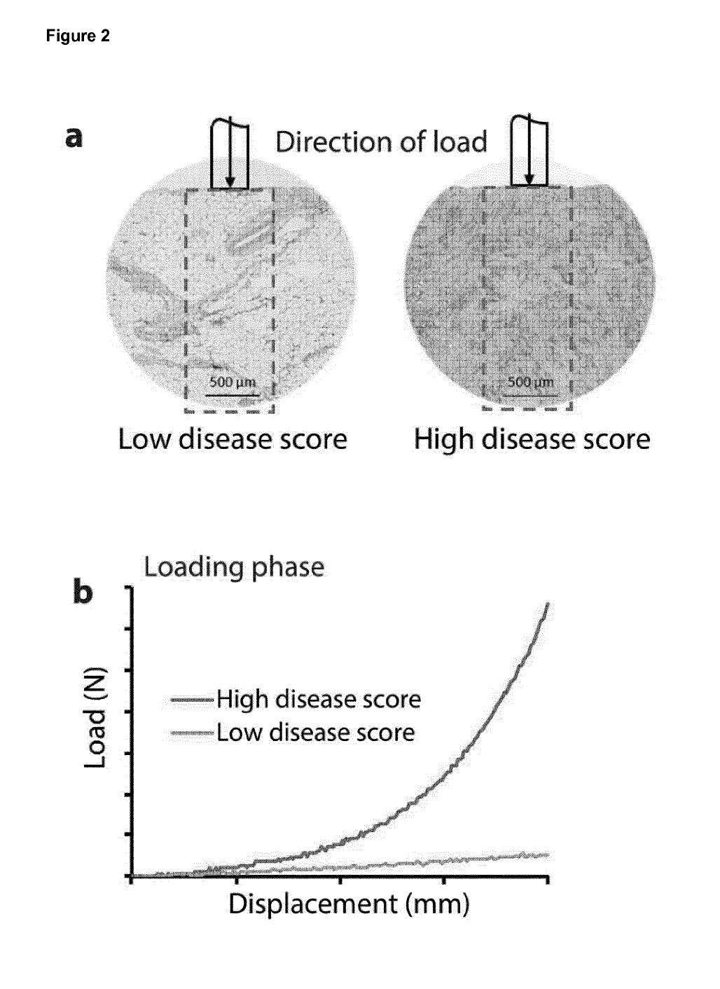

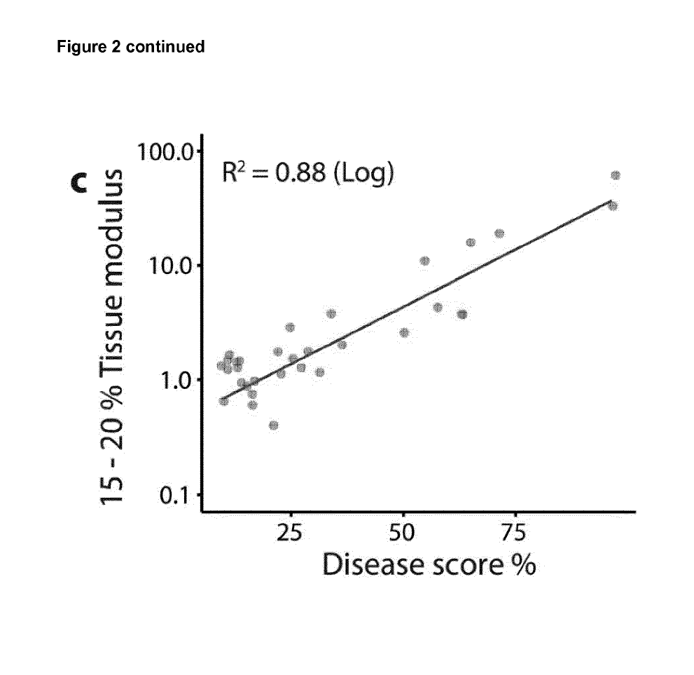

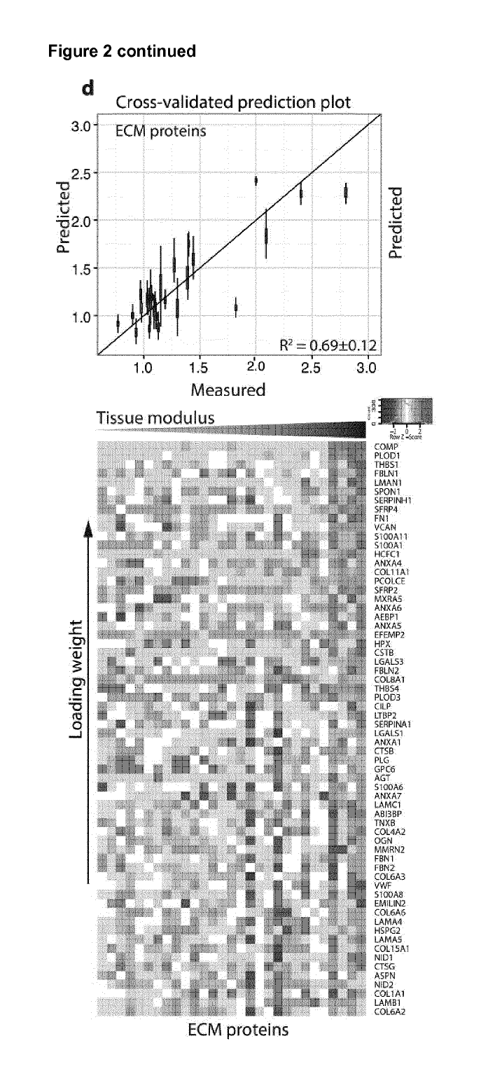

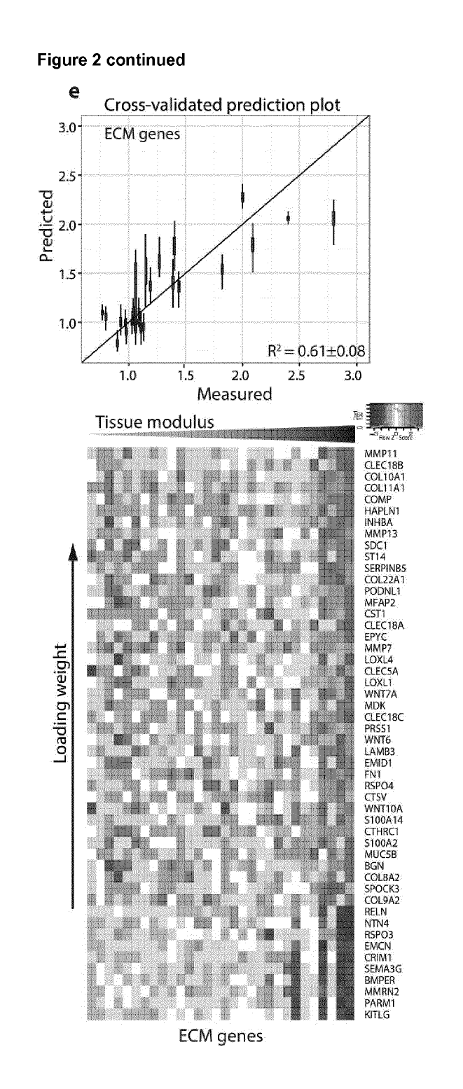

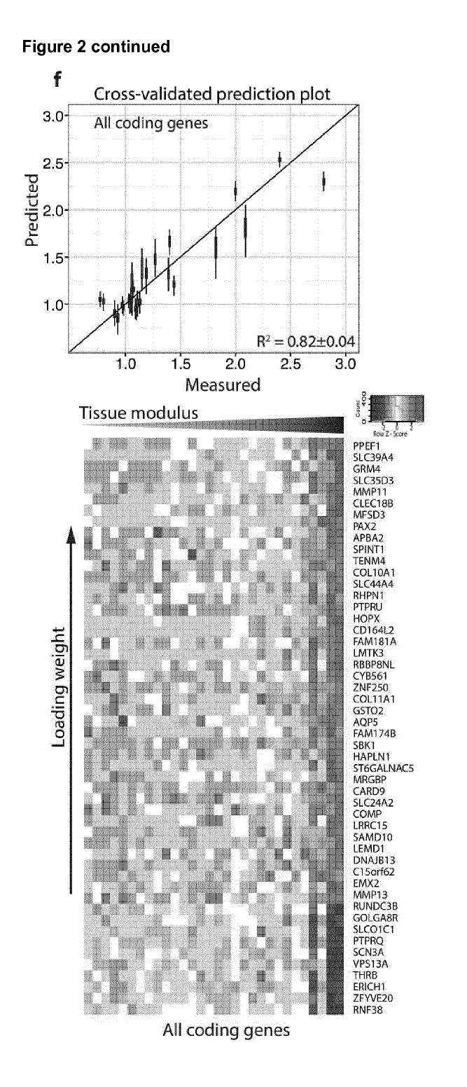

FIG. 2. Identification of Molecular Components that define Tissue Modulus

a) Orientation of flat-punch indentation showing representative low and high disease score samples, dashed line indicates tissue area analysed for determining disease score. b) Representative load-displacement curve from loading phase obtained from high and low disease score samples. c) Optimal tissue modulus correlated against combined % tumor plus stroma (disease score) (N=32, p<0.05). d-f) Crossvalidation plot of measured versus predicted tissue modulus values (diagonal line represents measured=predicted) and heatmap of PLS-identified d) matrisome proteins, e) matrisome genes, and f) all coding gene components that describe tissue modulus. Heatmap columns correspond to individual samples ordered by increasing tissue modulus. (N=29, 30 and 30, respectively). Rows ordered by decreasing model weight values.

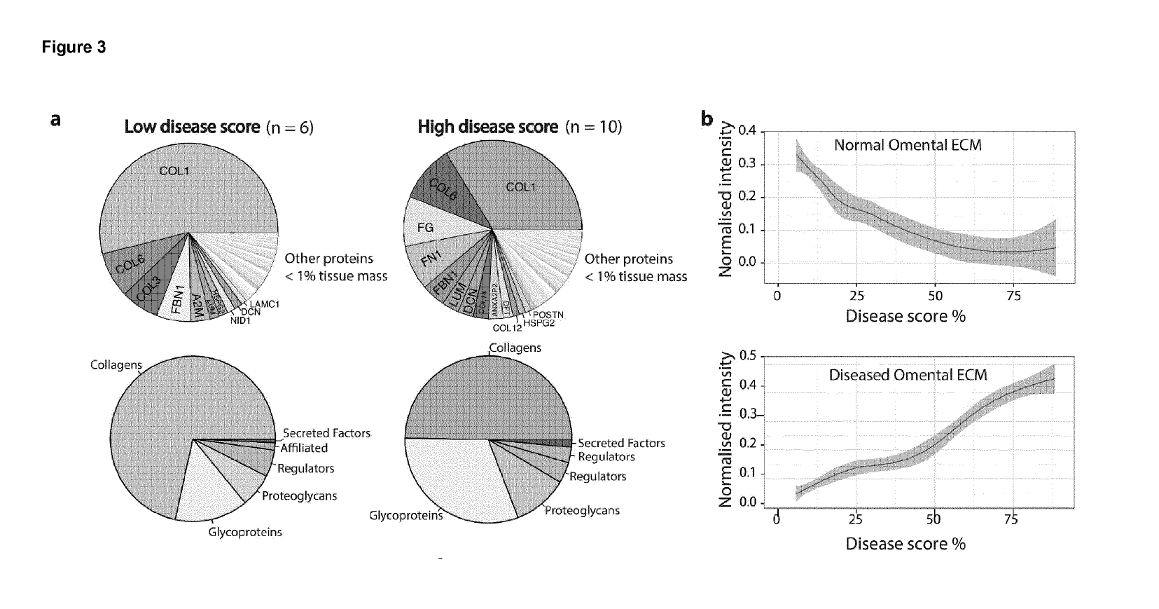

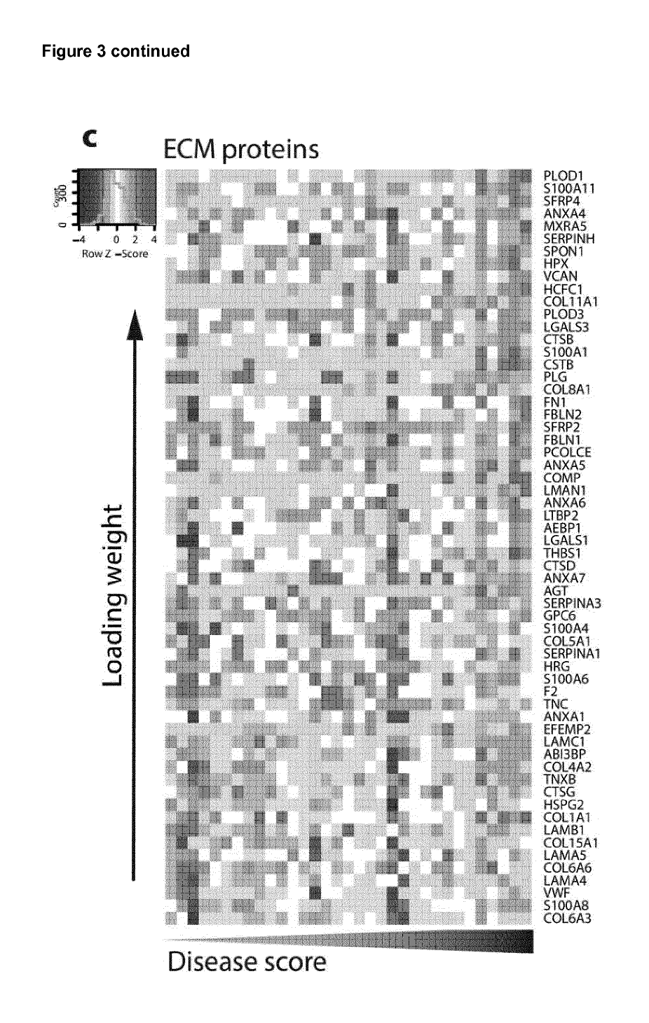

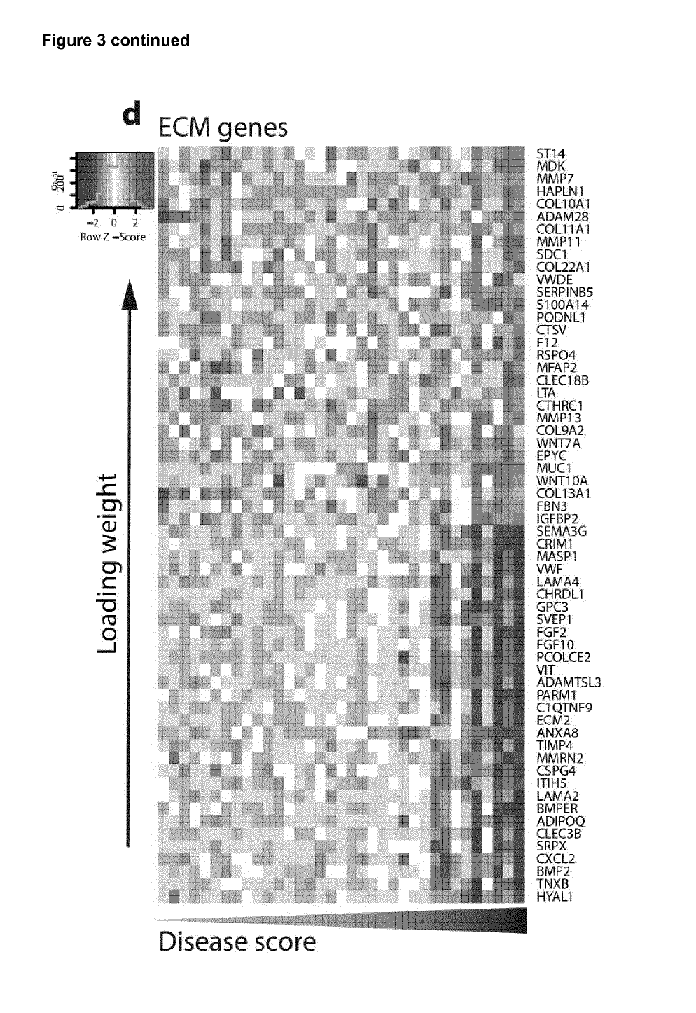

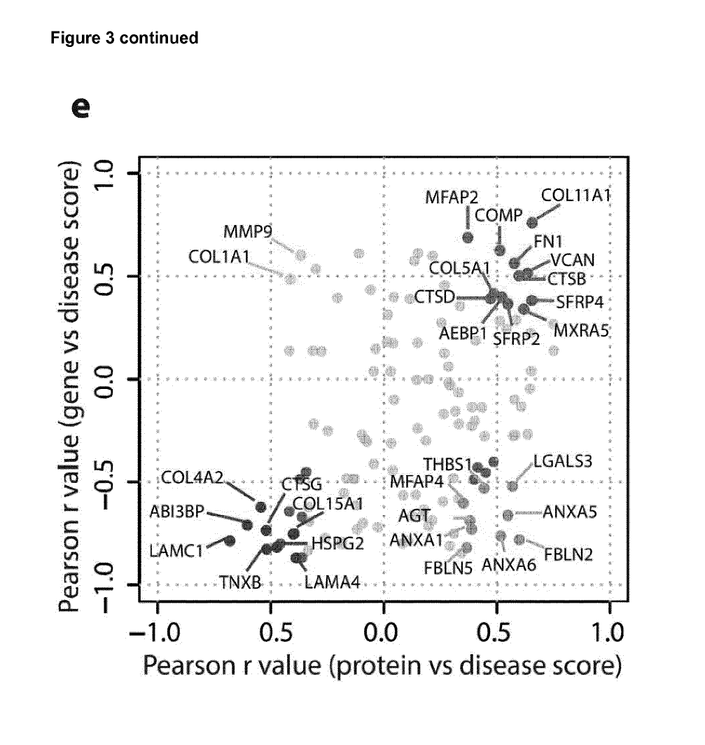

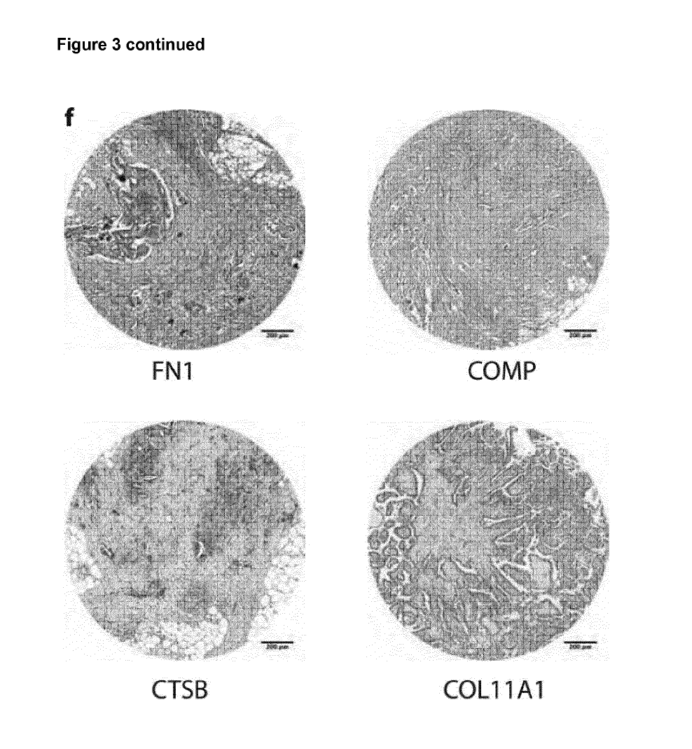

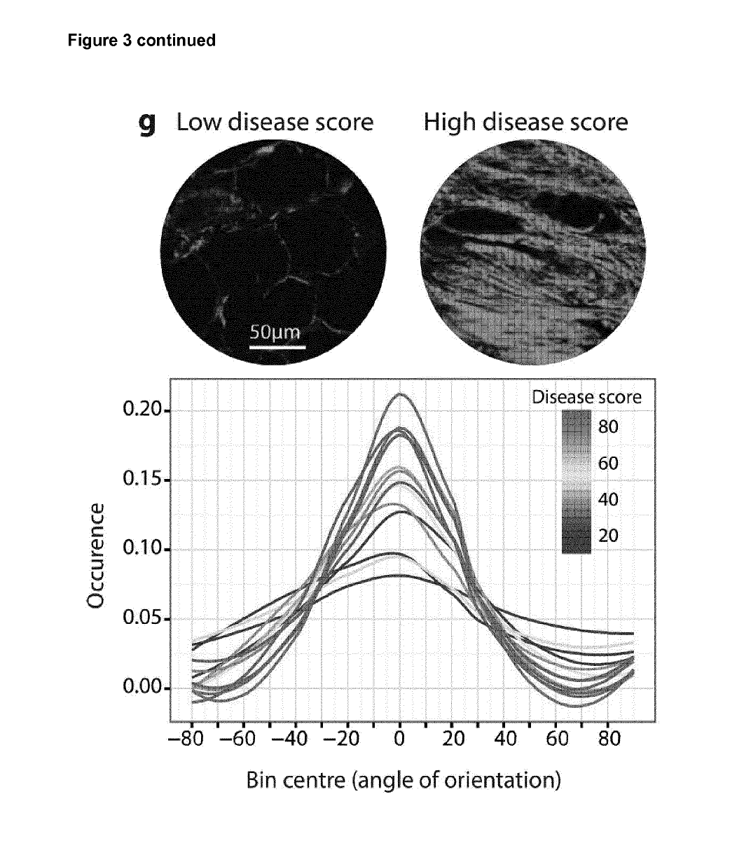

FIG. 3. Identification of ECM Proteins and Genes that define Tissue Architecture

a) Matrisome data displayed as relative mass ratios. Top panels show individual ECM proteins identified in low and high disease score tissue, bottom panels show the relative proportions of each of the major classes of ECM proteins in lowest (N=6) versus highest disease score (N=10). b) Line graphs illustrating normalized protein abundance and local polynomial regression fitted trend lines of proteins that either decrease (top panel), or increase (bottom panel) with disease score. c) PLS-identified ECM proteins and d) ECM genes that define disease score. e) Scatter plot of gene and protein correlation with disease score, highlighted molecules denote significant correlations (Pearson's correlation, N=33, p<0.05). f) Immunohistochemistry staining for four ECM proteins identified from PLS analysis as highly significantly related to disease score. g) Collagen fiber alignment; top panel shows representative images of high and low disease score tissue sections visualised using second harmonic generation, and bottom panel, semi quantification of fiber alignment from images plotted as number of fiber occurrences per angle bin (predominant fibre direction normalized to 0 degrees) with local polynomial regression fitted lines and disease colour coding. Scale-bars in f) 200 μm.

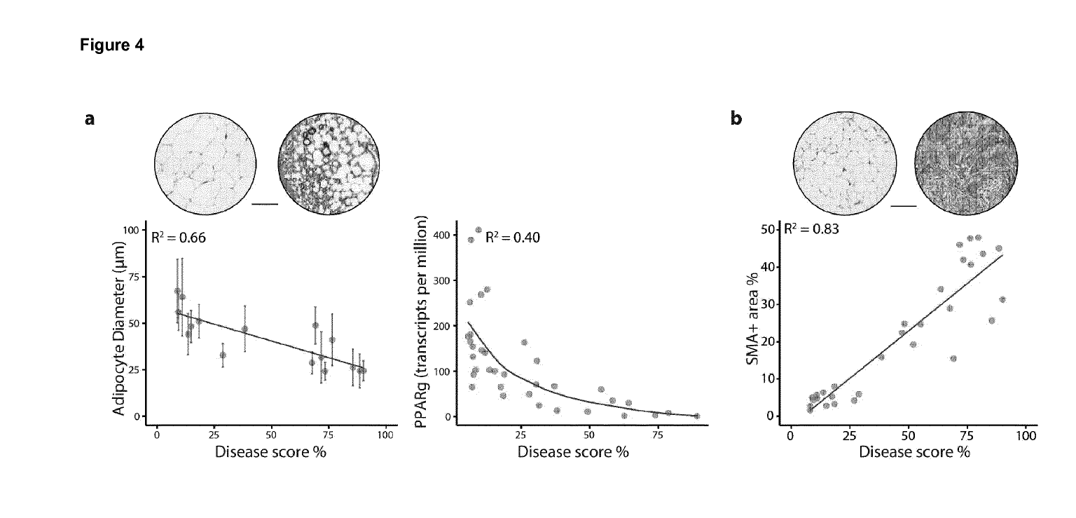

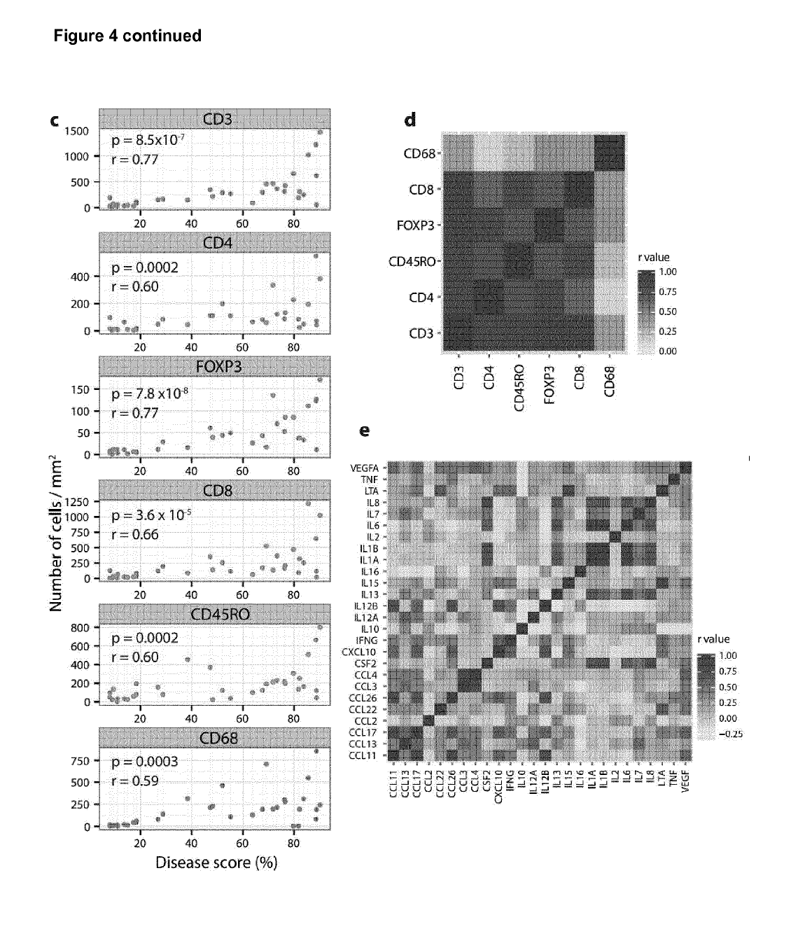

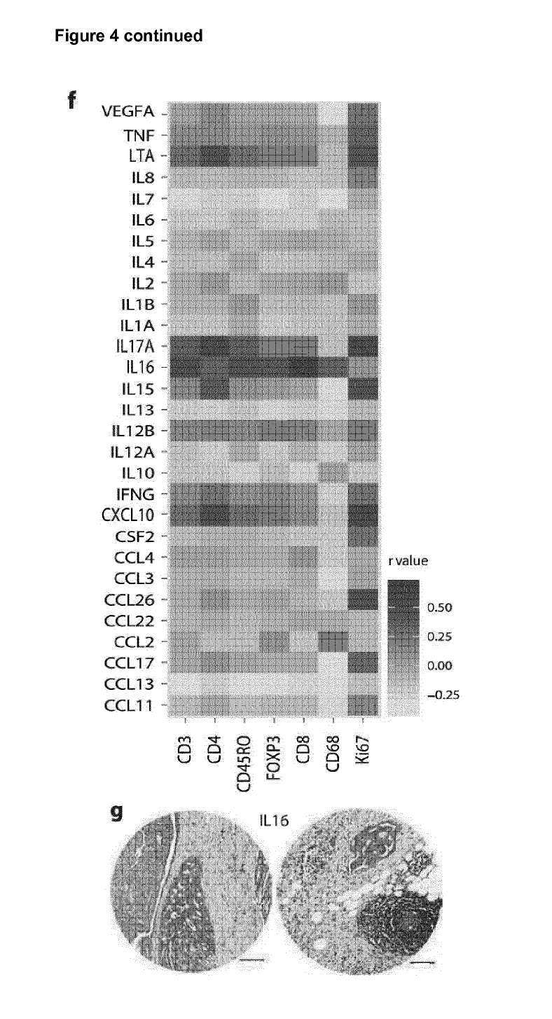

FIG. 4. The Cells of the TME Change with Disease Score and Tissue Modulus

a) Adipocyte diameter negatively correlated with increasing disease score. Top panel, representative low and high disease score tissue sections (stained for α-SMA) showing adipocytes. Scale-bars correspond to 100 μm. Bottom left panel, scatter plot illustrating mean±sd of digitally quantified adipocyte diameter (linear regression, N=16, R2=0.66, p=0.0001). Bottom right panel, scatterplot illustrating the correlation of PPARγ gene expression (tpm) against disease score (polynomial regression, N=35, R2=0.40, p<0.0001). b) Correlation of α-SMA positive cells against disease score. Top panel, representative low and high disease score tissue sections stained for α-SMA. Scale-bars correspond to 100 μm. Bottom panel, quantification of α-SMA+ area % against disease score (linear regression, N=30, R2=0.83, p<0.0001). c) Cleveland plots of immune cell counts against disease score (spearman's correlation, N=34). d-f) Heatmap of pairwise pearson's correlation coefficients of d) immune cell counts (N=34), e) MSD-quantified cytokine/chemokine (N=32) and f) MSD-quantified cytokine/chemokine correlations against immune cell counts (N=32). g) IHC of IL16 in HGSOC omental biopsies. Scale-bars correspond to 100 μm.

FIG. 5. A Matrix Signature that Predicts Survival in Ovarian Cancer.

a) Venn diagram showing the overlap of PLS-identified molecules associated to tissue modulus and disease score (DS) at both gene and protein level. A total of 22 ECM-associated molecules overlapped across all analyses, red (darker) colour denotes positive association and blue (lighter) colour negative association of each molecule at gene (G) and protein (P) level with disease score and tissue modulus. b) Network of known protein:protein interactions from IntAct and BioGRID within the 22 ECM-associated. Visualisation was carried out using Cytoscape v.3.3.0. c) Based on gene expression levels of these molecules the inventors calculated a matrix index as the ratio of average level of expression of genes positively associated to those negatively associated with disease score and tissue modulus. Scatter plots show the correlation of matrix index with tissue modulus (linear regression, N=30, R2=0.74, p<0.0001) and disease score (linear regression, N=35, R2=0.76, p<0.0001). d) Association of matrix index with immune gene signature expression. Barplot illustrates Spearman p-values, FDR corrected using the Benjamini & Hochberg method. Red (top 7 bars) denotes positive correlations, blue (bottom 4 bars) denotes negative and gray (middle bars) denotes insignificant associations. The dotted line specifies the significance cutoff p=0.05. e) Kaplan-Meier survival curves with overall survival of TOGA and ICGC dataset for HGSOC divided by high or low matrix index. The x-axis is in the unit of years. f) Comparison of hazard ratio scores (HR, with 95% CI) derived from Cox proportional hazards model for matrix index and the indicated gene expression signatures extracted from literature on the ovarian TCGA dataset. Left panel corresponds to univariate analysis, right panel corresponds to multivariate analysis taking into account age, tumor stage, grade and treatment (i.e., primary therapy outcome success). The asterisks represent the significance in the KM analysis between the high- and low-index groups (***p<0.001, **p<0.01, *p<0.05 and ▪0.05<p<0.1).

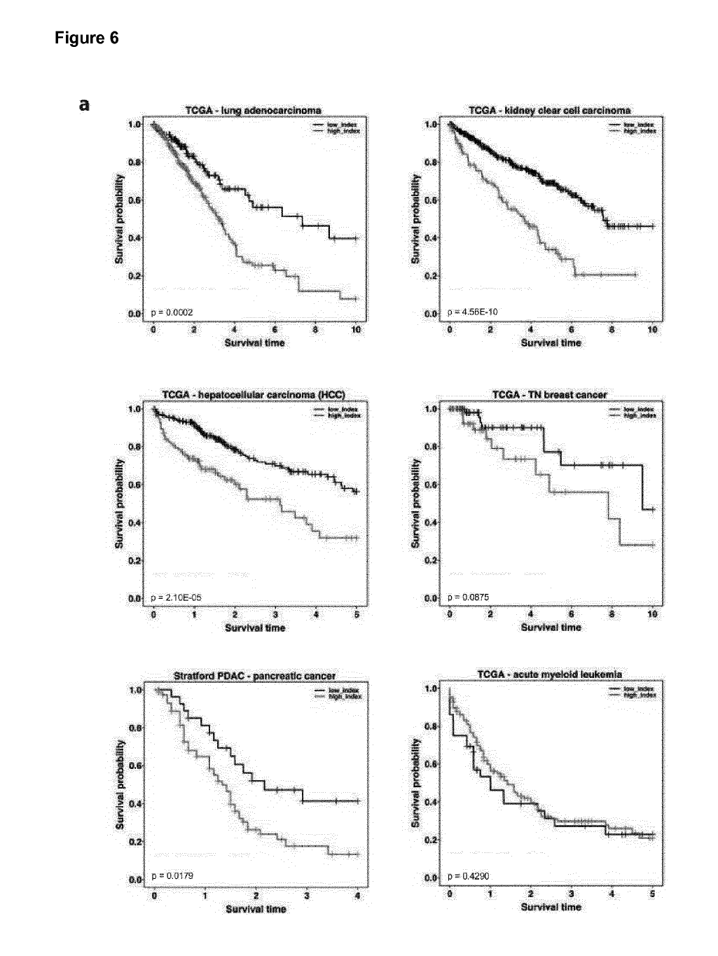

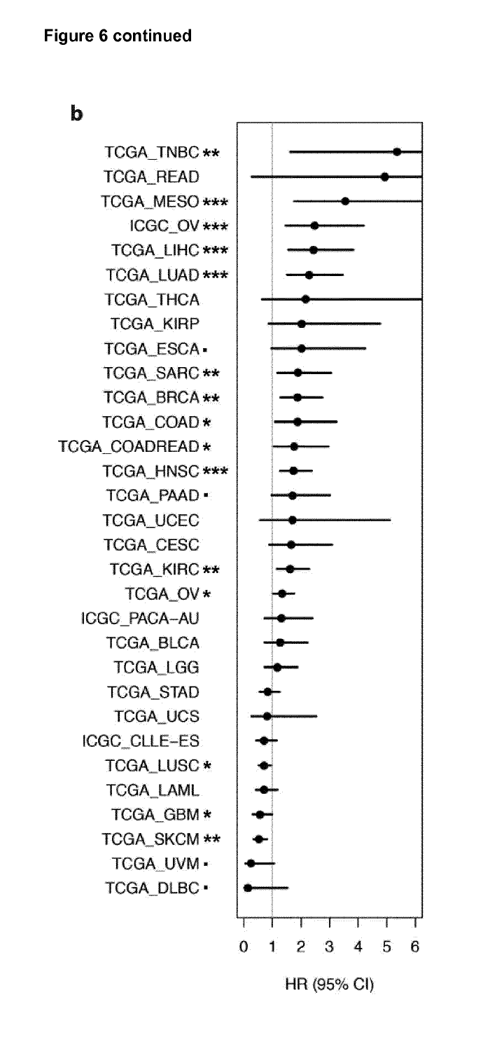

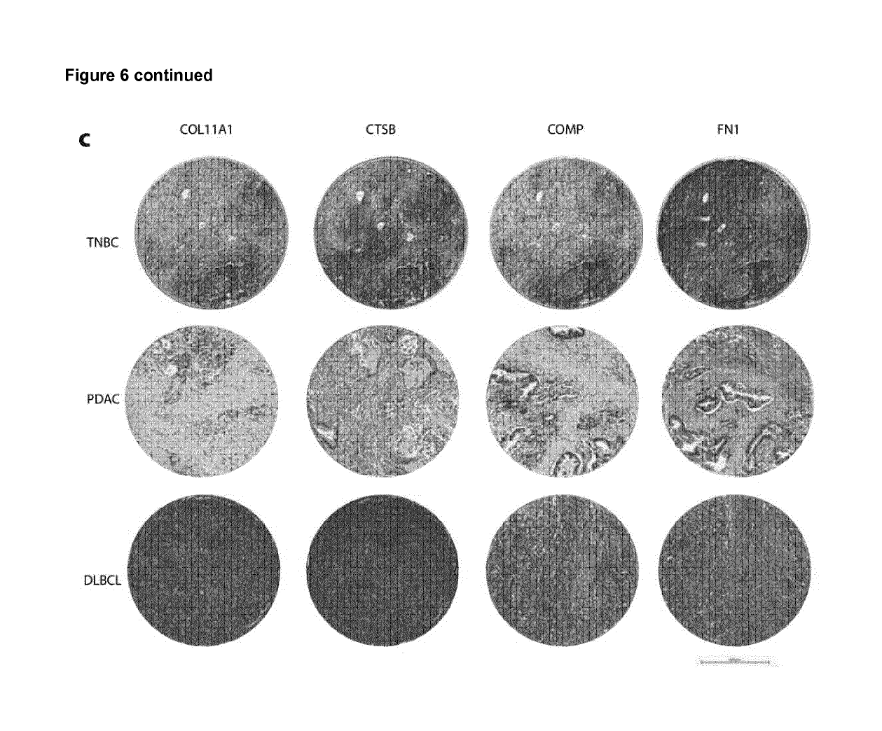

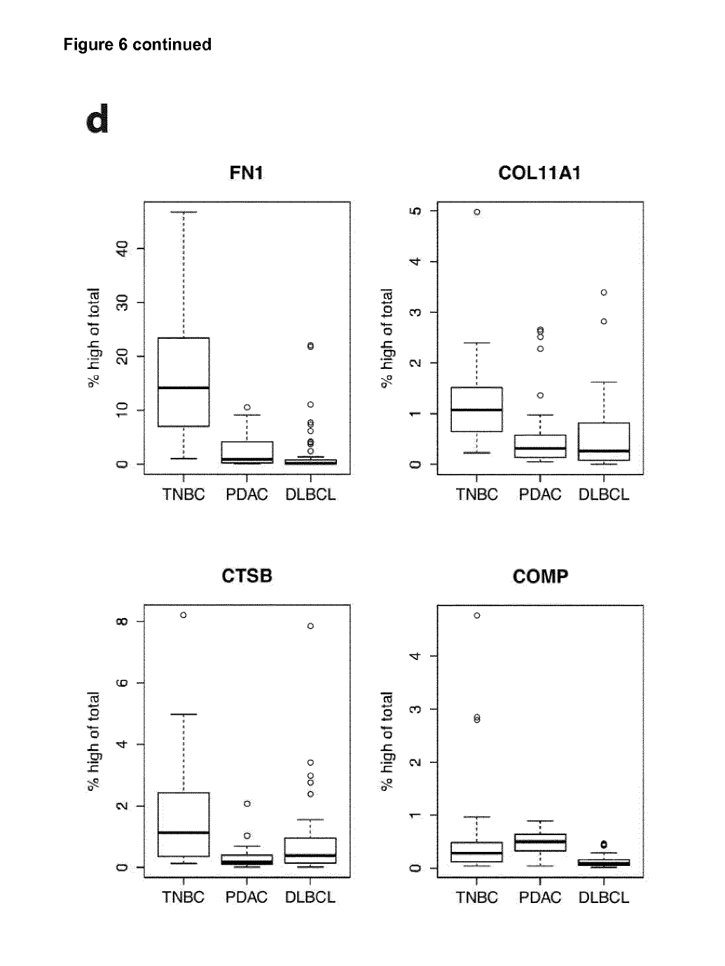

FIG. 6. Matrix Index Reveals a Common Stromal Reaction Across Cancers

a) Kaplan-Meier survival curves with overall survival from the indicated datasets divided by high or low matrix index. The x-axis is in the unit of years. b) Multivariate hazard ratio (HR, with 95% CI) derived from a Cox proportional hazards regression model across cancer types/datasets using the matrix index. In each cancer, patients were split into high and low index groups, and their association with the overall survival (OS) was tested taking into account age, stage, grade (T-factor), and treatment factors. Asterisks represent the significance in the KM analysis between the high- and low-index groups (***p<0.001, **p<0.01, *p<0.05 and ▪0.05<p<0.1). HR>1 means that high index is inversely correlated with OS, while HR<1 means high index positively correlated OS. c) Example IHC images digitally quantified using definiens on cancer tissue array cores for matrix index proteins FN1, COL11A1, CTSB, and COMP. High intensity staining=red, medium=orange, low=yellow. d) Quantification of IHC staining on tissue arrays using Definiens software. Box plots illustrate the percentage area of high intensity staining for each marker. Scale bar=500 μm. COL11A1 and FN1, N=30, 36, 54; CTSB, N=28, 35, 52; COMP, N=29, 35, 54; for TNBC, PDAC and DLBCL respectively.

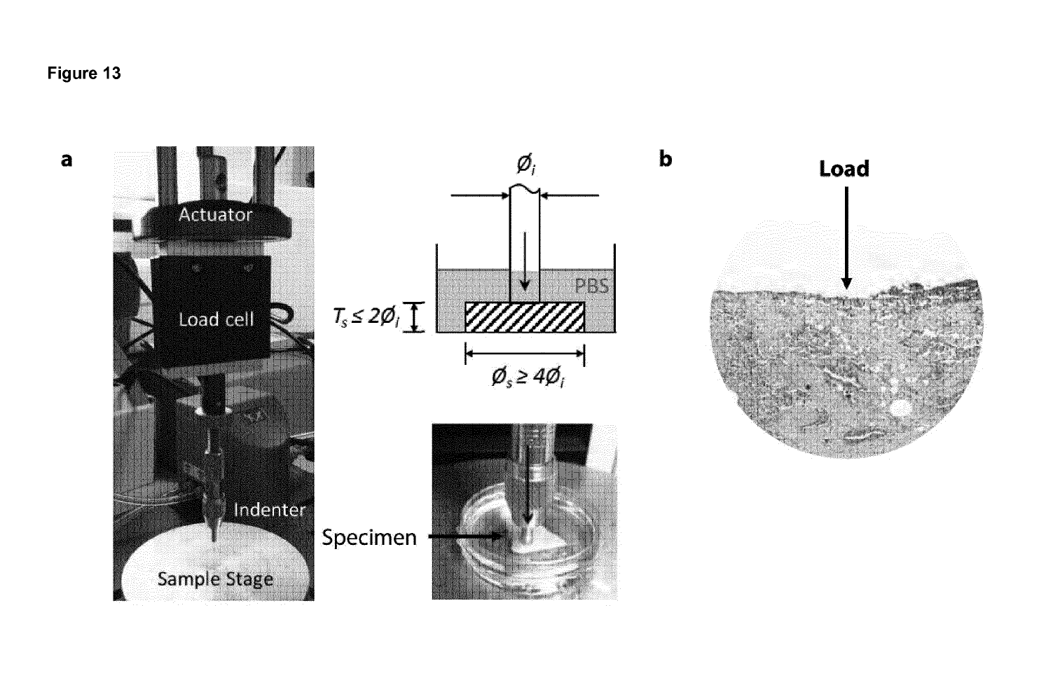

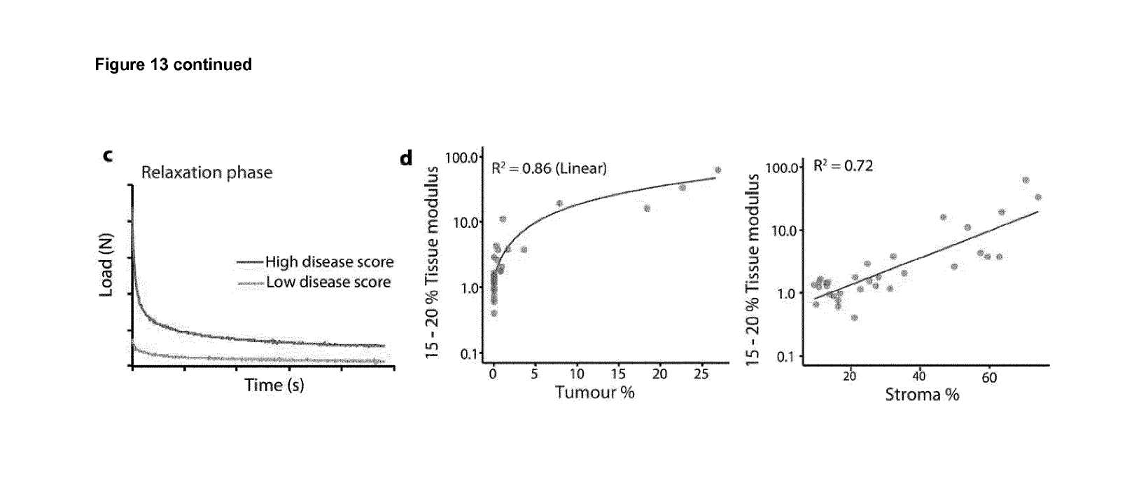

FIG. 13. Overview of the Biomechanical Approach taken to Quantify Tissue Modulus.

a) Setup of flat-punch indentation technique; left panel shows image of actuator driven flat-punch indenter connected to a load cell; top right panel shows a schematic of the relationship between the indenter diameter, Øi, and the test specimen thickness, Ts, and diameter, Øs, while loaded (direction indicated by vertical arrow) in phosphate buffered saline (PBS); bottom right panel shows a test in progress. b) A representative cross-section taken from a test specimen cut perpendicular to the direction of load (arrow) under the area of flat-punch contact marked by green tissue dye. c) Representative load-displacement curve from relaxation phase obtained from high and low disease score samples. d) Optimal tissue modulus correlated against % tumor and % stroma N=32, p<0.05).

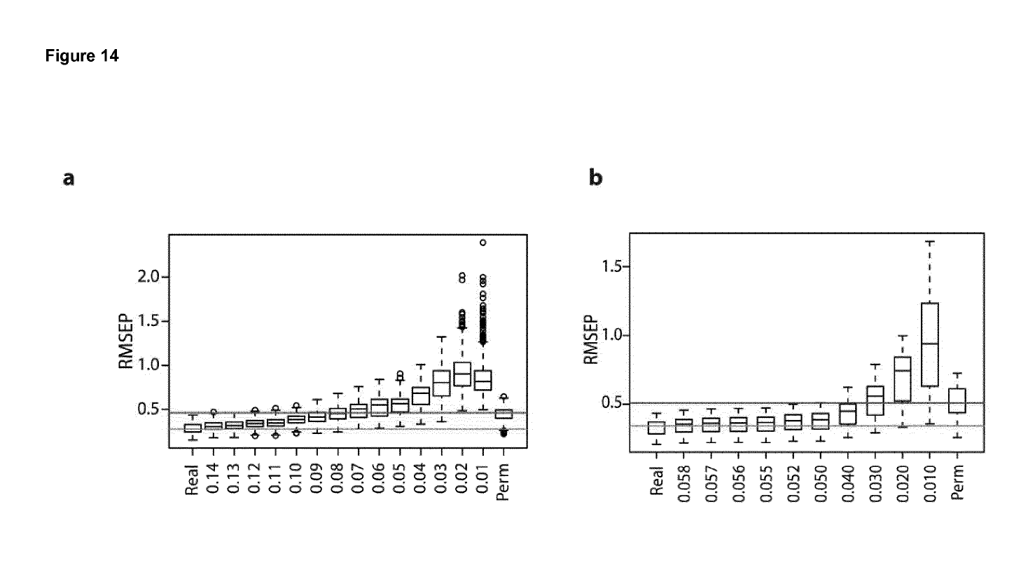

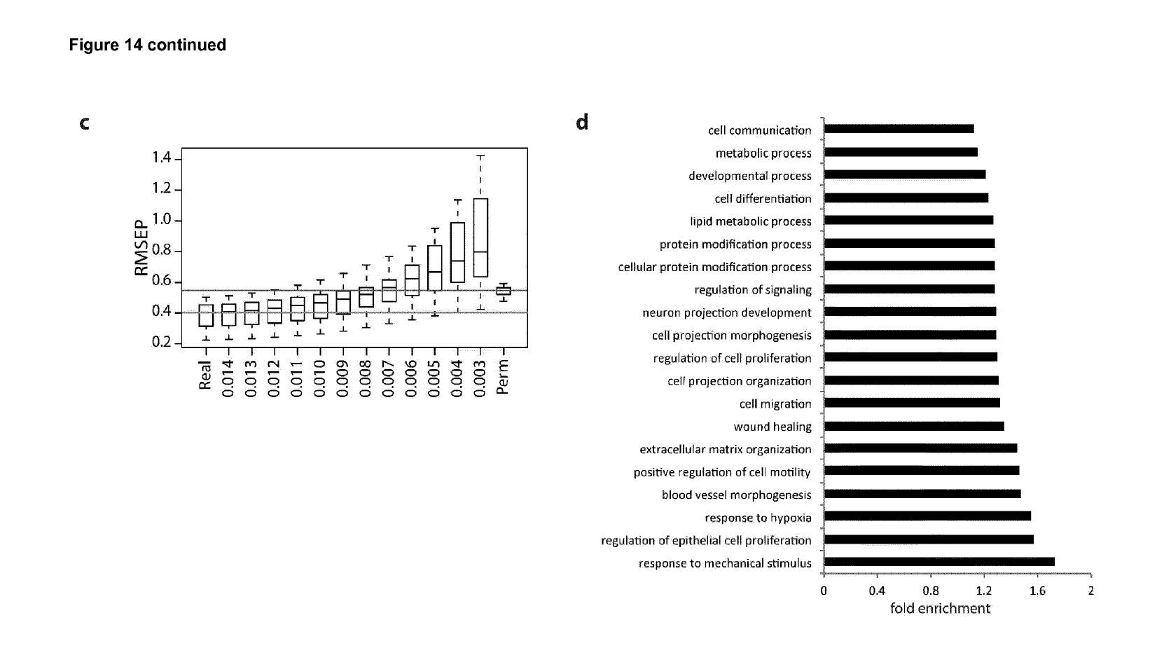

FIG. 14. Analysis used to Identify Components Associated with Tissue Modulus.

a-c) Permutation-derived threshold for determining sets of molecular components significantly associated with tissue modulus. Boxplots illustrate bootstrapped RMSEP values on cross-validated PLS regression models of a) ECM associated protein versus tissue modulus b) ECM-associated genes versus tissue modulus, c) all coding genes versus tissue modulus. In each case, bootstrapped RMSEP of the complete dataset as well as following exclusion of variables in order of weight and of a permuted dataset is illustrated. Green line denotes median RMSEP of the complete dataset; red line denotes median RMSEP of the permuted dataset and was used as a cutoff value. d) Significantly enriched Biological Process Gene Ontology terms in PLS identified protein coding genes (7,287) correlative to tissue modulus (p<0.05).

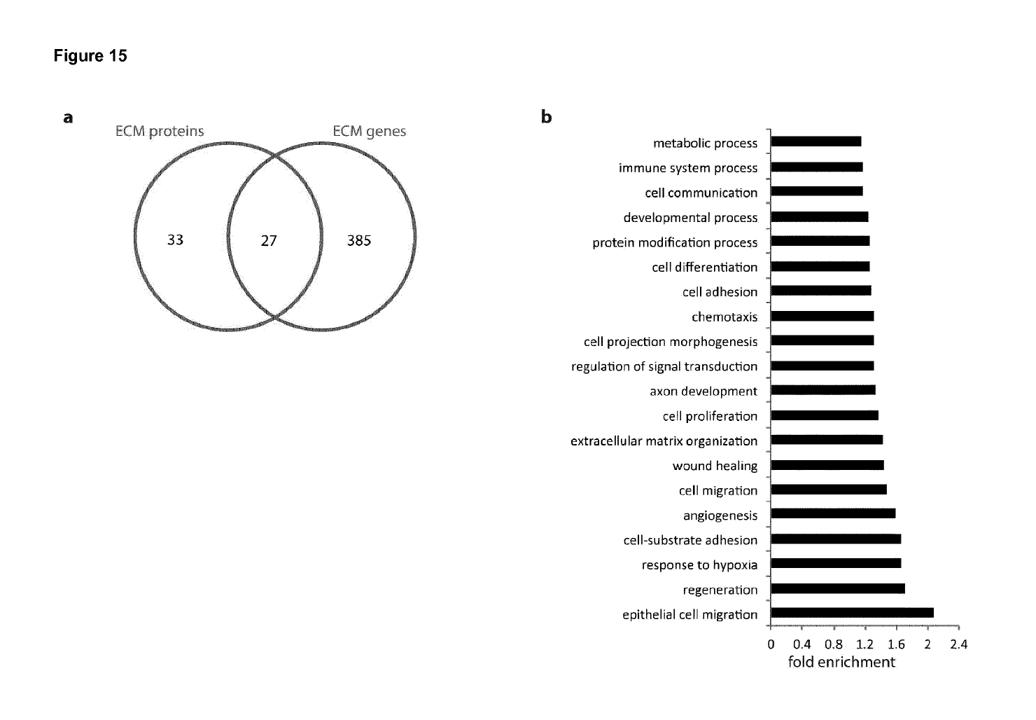

FIG. 15. Analysis of PLS-Identified ECM Proteins and Genes.

a) Venn diagram showing the overlap of ECM-associated genes and ECM-associated proteins identified by PLS regression models as significantly associated with disease score. Note this figure only considers association with disease score and not also tissue modulus, and so is less reliable that the smaller 22 gene panel, which was determined by association with both disease score and tissue modulus. b) Significantly enriched Biological Process Gene Ontology terms in PLSidentified protein coding genes (7,380) correlative to disease score (p<0.05).

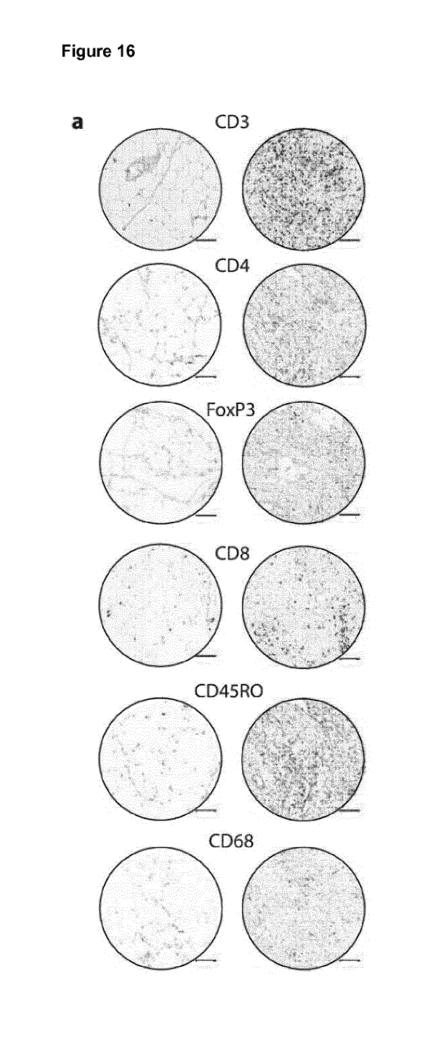

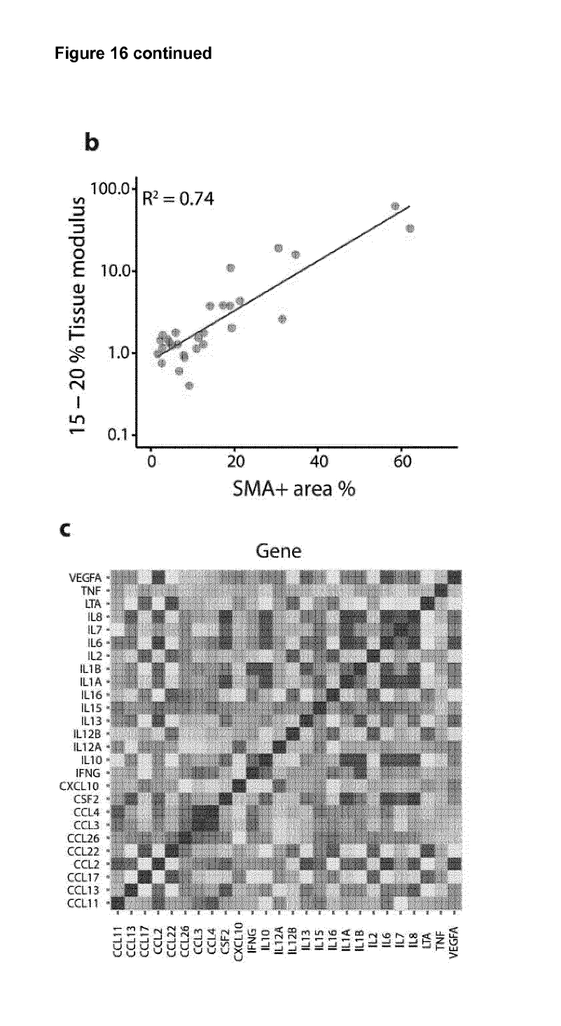

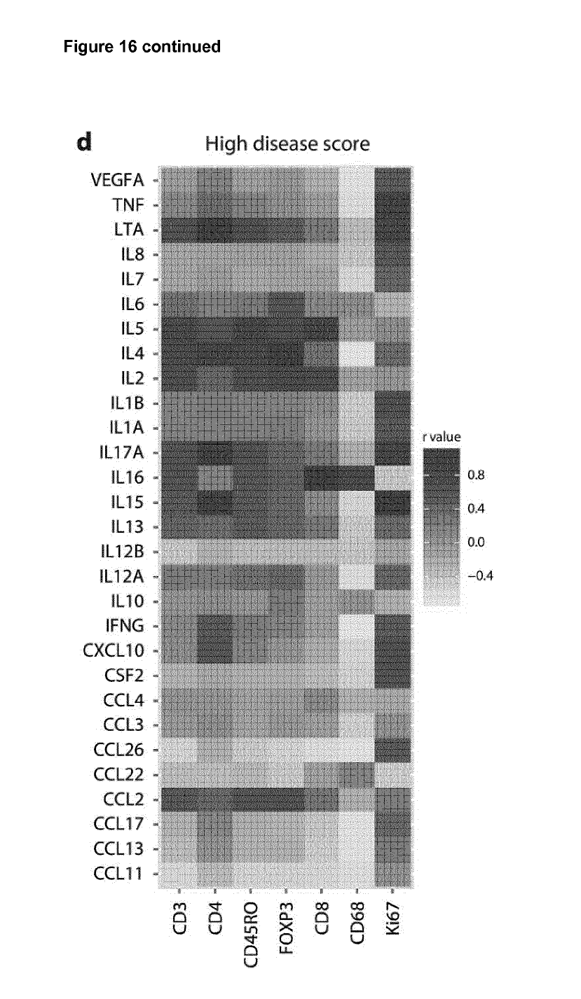

FIG. 16. Immune Cells and Cytokines of the Tumor Microenvironment

a) Representative immunohistochemistry images of low and high disease score tissue sections stained for the indicated markers. Scale-bars correspond to 100 μm. b) Correlation of tissue modulus against α-SMA+ area on tissue sections (linear regression, N=29, R2=0.74, p<0.0001). c) Heatmap of pairwise pearson's correlation coefficients of MSD-quantified cytokine/chemokine gene expression (tpm). d) Heatmap of pairwise pearson's correlation coefficients of MSD-quantified cytokine/chemokine correlations against immune cell counts in the top 10 highest disease score samples.

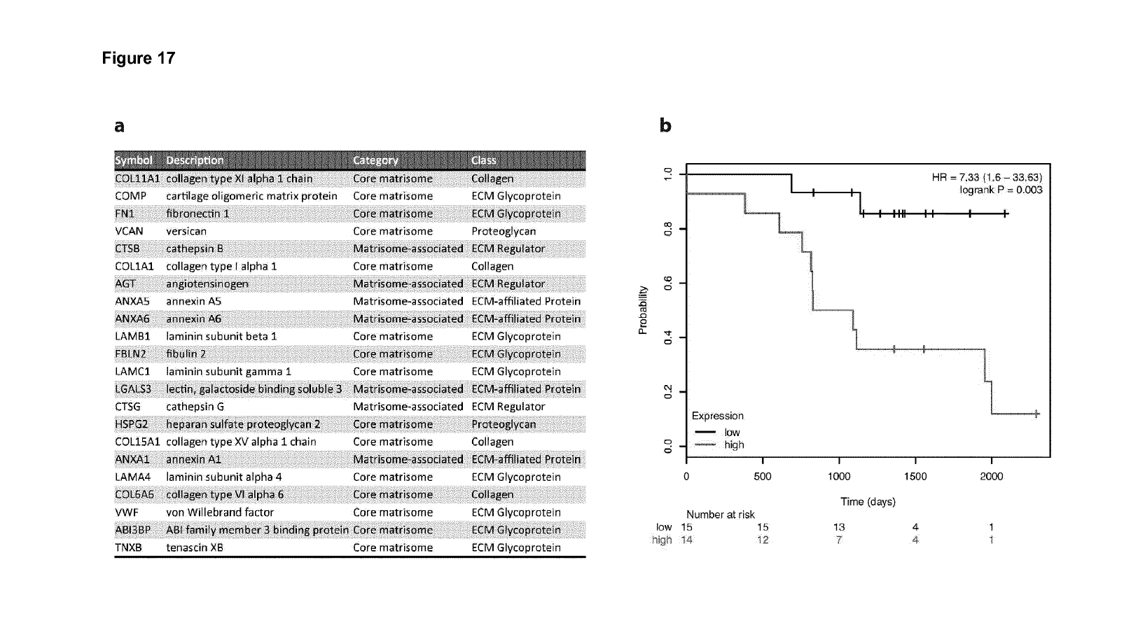

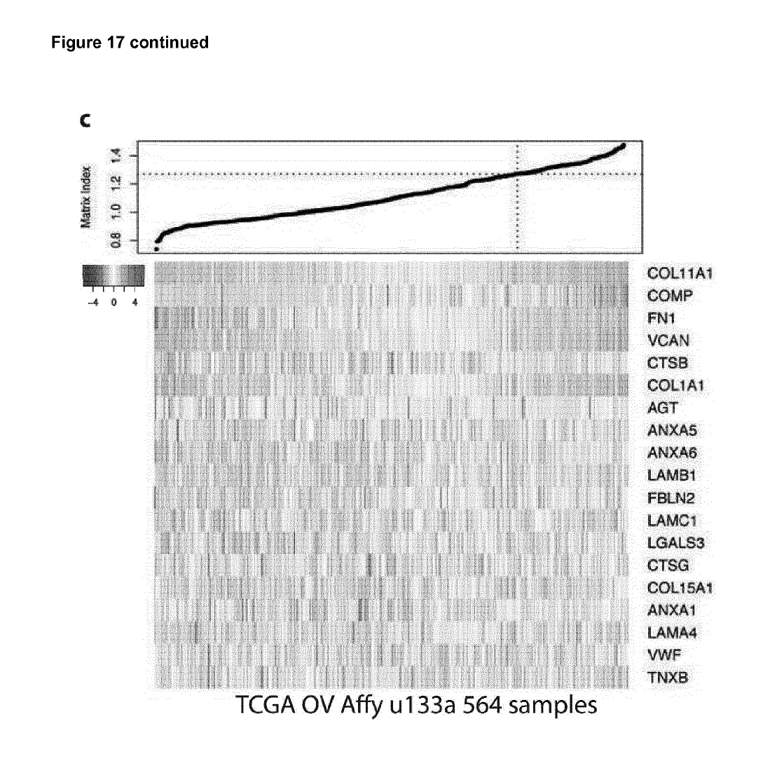

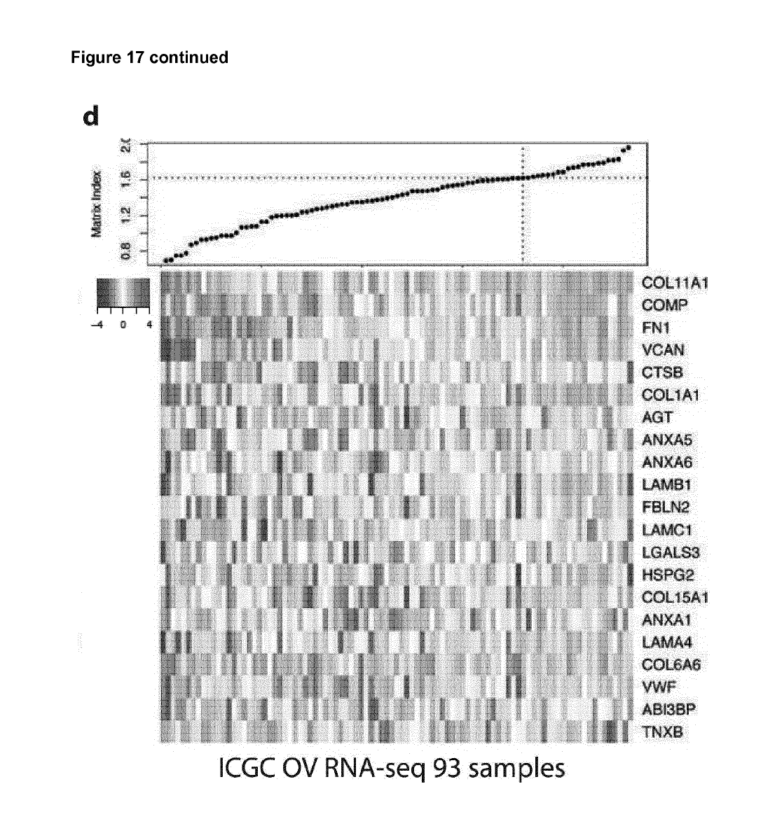

FIG. 17. The Matrix Index Signature

a) Description of gene, matrisome category and class of the 22-matrix molecules. b) Kaplan-Meier survival curve with overall survival divided by high or low matrix index derived from the present study's transcriptomic dataset. c, d) Matrix index values and expression heatmap of matrix index genes detected across patient samples of the c) TCGA OV Affy u133a and d) ICGC OV RNA-seq datasets. Dotted lines denote the cut-off value of high and low index patient groups.

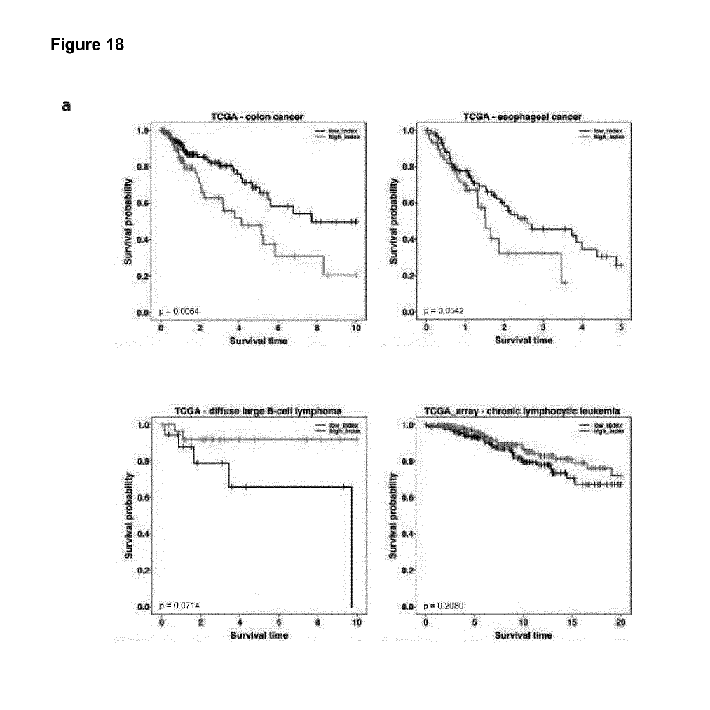

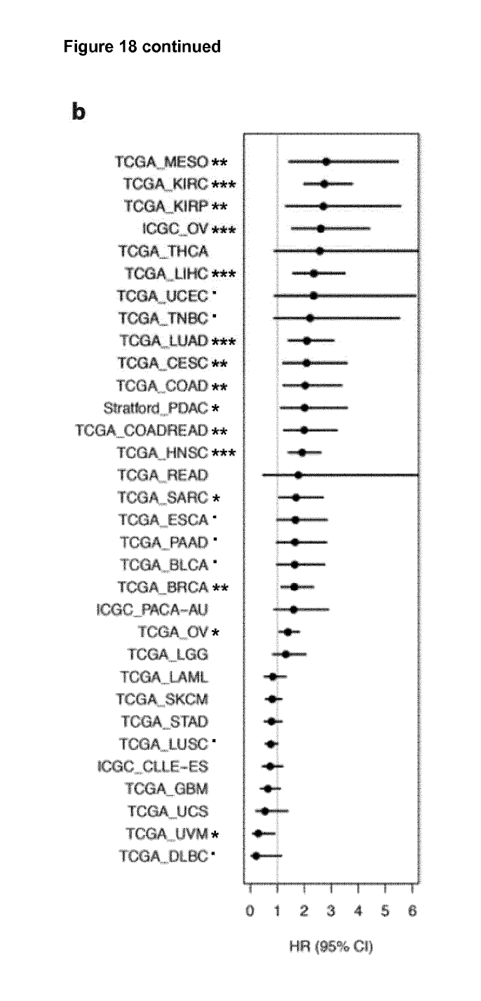

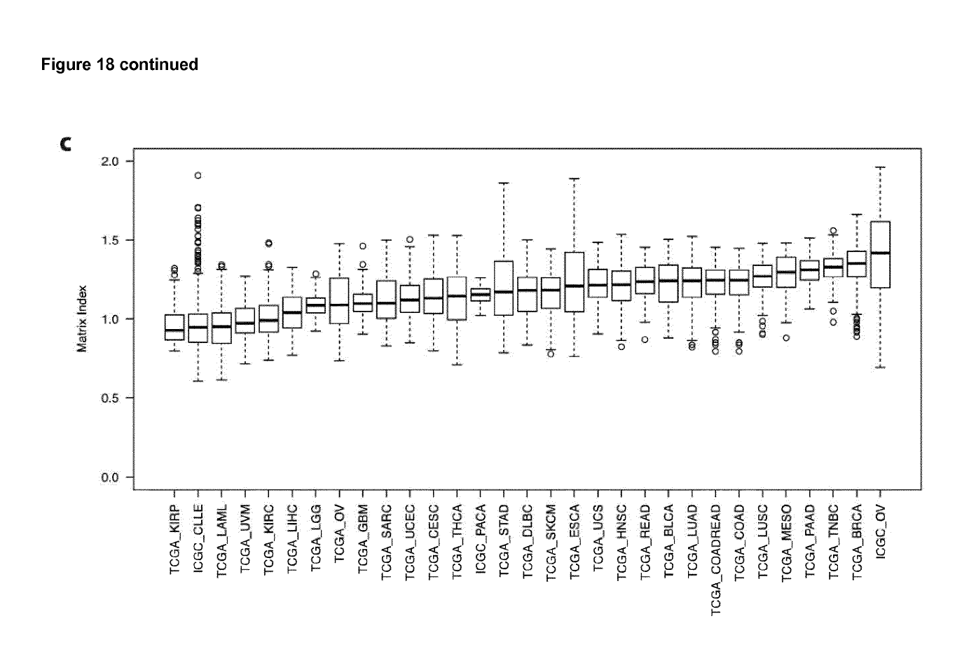

FIG. 18. The Matrix Index in other Cancers

a) Kaplan-Meier survival curves with overall survival from the indicated datasets divided by high or low matrix index. The x-axis is in the unit of years. b) Univariate hazard ratio (HR, with 95% CI) derived from a Cox proportional hazards model across cancer types using the matrix index. In each cancer, patients were split into high and low index groups, and their association with the overall survival (OS) was tested. The asterisks represent the significance in the KM analysis between the highand low-index groups (***p<0.001, **p<0.01, *p<0.05 and ▪0.05 <p <0.1). HR>1 means that high index is inversely correlated with OS, while HR<1 means high index positively correlated OS. c) Distribution of matrix index across cancer datasets by boxplots.

### EXAMPLES

Methods

Ovarian Cancer Patient Samples

Patient samples were kindly donated by women with high-grade serous ovarian cancer (HGSOC) undergoing surgery at Barts Health NHS Trust between 2010 and 2014. Blood and tissue that was deemed by a pathologist to be surplus to diagnostic and therapeutic requirement were collected together with associated clinical data under the terms of the Barts Gynae Tissue Bank (HTA licence number 12199. REC no: 10/H0304/14).

RNA Isolation

Whole tissue. Total RNA was extracted from 10×50 μm cryosections from frozen tissue sections and placed directly into the RLT Plus buffer (Qiagen) and rigorously vortexed. Samples were then processed using Qiagen RNeasy Plus Micro kit according to manufacturer's instructions.

Laser-capture microscopy (LCM). Membrane coated microscope slides (MembraneSlide 1.0 PEN from Zeiss) were activated under UV for 30 min. Frozen tissue sections were cut at a thickness of 15 μm onto the membrane slides, which werestored on dry-ice for up to 3 h. The sections were stained with hematoxylin and immediately washed in distilled water followed by tap water. They were then dehydrated by submerging in 70% ethanol for 30 sec, 100% ethanol for 1 min, and xylene for 30 sec. The sections were air-dried and kept on dry-ice until processed. A Zeiss PALM Microbeam laser capture microscope system was used to dissect tumour islands and surrounding stroma. A total of six sections per sample were dissected and total RNA was isolated using the Qiagen RNeasy Plus Micro kit according to manufacturer's instructions. Laser-captured RNA samples were further processed prior to sequencing using SMARTer RNA amplification.

RNA quality analysis. Total RNA isolated from whole tissue and laser-captured samples were analyzed on agilent bioanalyzer 2100 using RNA PicoChips according to manufacturer's instructions. RNA integrity numbers (RIN) between 8.1 and 9.9 were found from whole tissue and 7.2 to 7.8 for laser-captured samples.

RNA Sequencing and Analysis

RNA-Seq was performed by Oxford Gene Technology (Benbroke, UK) to ˜42× mean depth on the Illumina HiSeq2500 platform, strand-specific, generating 101 bp paired end reads, as previously described (Boehm et al.⁴⁸). RNA-Seq reads were mapped to the human genome (hg19, Genome Reference Consortium GRCh37) using RSEM version 1.2.4¹ in dUTP strand-specific mode. Bowtie version 0.12.7² was used to perform the mapping as part of the RSEM pipeline. The number of reads aligned to the exonic region of each gene was counted based on Ensembl annotations. Only genes that achieved at least 10 reads per sample were kept. Log₂ counts per million (cpm) were calculated using the edgeR package (version 3.8.6)³. RNA-Seq data have been deposited in Gene Expression Omnibus (GEO) under the accession number GSE71340.

Proteomics

Enrichment for ECM-component: The ECM component was enriched from frozen whole tissue sections (20×30 μm sections, approximately 40-50 mg of tissue) as previously described⁴ using a CMNCS extraction kit (Stratech). Briefly, tissue sections were homogenized in buffer C (250 μL per sample) by vortexing for 2 min per sample then incubating for 20 min, 4° C., with agitation. The samples were centrifuged at 18000 g for 20 min at 4° C. and the supernatants were stored at −20° C. This fraction was analyzed for cytokine and chemokine content using the mesoscale discovery platform (see separate method section below). The samples were then washed with buffer W (300 μL per sample), quickly vortexed and then centrifuged at 18000 g for 20 min, 4° C. The supernatants were removed and the pellets resuspended in buffer N (150 μL per sample), incubated for 20 min, 4° C., with agitation and centrifuged at 18000 g for 20 min, 4° C. Supernatants were discarded and this step was repeated. Pellets were then resuspended and well-mixed in buffer M (100 μL per sample), incubated for 20 min, 4° C., with agitation and then centrifuged at 18000 g for 20 min, 4° C. The supernatants were discarded and the pellets were then resuspended and well-mixed in buffer CS (200 μL per sample, pre-heated at 37° C.), incubated for 20 min at room temperature, with agitation and centrifuged at 18000 g for 20 min, 4° C. The supernatants were discarded and the pellets resuspended and well-mixed in buffer C (150 μL per sample), incubated for 20 min, 4° C., with agitation and centrifuged at 18000 g for 20 min, 4° C. The pellets that remained at the end of 3 this process were enriched for extracellular matrix (ECM) proteins and stored at −80° C.

Peptide preparation: ECM enriched pellets were solubilised in 250 μL of an 8 M Urea in 20 mM HEPES (pH8) solution containing Na₃VO₄ (100 mM), NaF (0.5 M), β-Glycerol Phosphate (1 M), Na₂H₂P₂O₇ (0.25 M). Samples were vortexed for 30 sec and left on ice prior to sonication at 50% intensity, 3 times for 15 sec, on ice. Tissue lysate suspensions were centrifuged at 20000 g for 10 min, 5° C., and the supernatant recovered to protein low-bind tubes. BCA assay for total protein was then performed and 80 μg of protein was carried forward to the next step in urea (8 M, 200 μL per sample). Prior to trypsin digestion disulphide bridges were reduced by adding 500 mM Dithiothreitol (DTT, in 10 μL) to samples, which were then incubated at room temperature for 1 h with agitation in the dark. Free cysteines were then alkylated by adding 20 μL of a 415 mM iodacetamide solution to samples, which were again incubated at room temperature for 1 h with agitation in the dark. The samples were then diluted 1 in 4 with 20 mM HEPES. Removal of N-glycosylation was then achieved by addition of 1500U PNGaseF (New England Biolabs), then vortexing, and incubation at 37° C. for 2 h. 2 μL of a 0.8 μg/μL LysC (Pierce) per sample was then added, gently mixed and then incubated at 37° C. for 2 h. Protein digestion was achieved with the use of immobilized Trypsin beads (40 μL of beads per 250 μg of protein) incubated with the derivitised protein lysate for 16 h at 37° C. with shaking. Peptides were then de-salted using C-18 tip columns (Glygen). Briefly, samples were acidified with trifluoroacetic acid (1% v/v), centrifuged at 2000 g, 5 min, 5° C., before transferring the supernatant to a new microcentrifuge tube on ice. Glygen TopTips were washed with 100% ACN (LC-MS grade) followed by 99% H₂O (+1% ACN, 0.1% TFA) prior to loading the protein digest sample. The sample was washed with 99% H₂O (+1% ACN, 0.1% TFA), and the desalted peptides eluted with 70/30 ACN/H2O+0.1% FA. The samples were dried and stored at −20° C.

Mass Spectroscopy analysis and bioinformatics: Dried samples were dissolved in 0.1% TFA (0.5 μg/μl) and run in a LTQ-Orbitrap XL mass spectrometer (Thermo Fisher Scientific) connected to a nanoflow ultra-high pressure liquid chromatography (UPLC, NanoAcquity, Waters). Peptides were separated using a 75 μm×150 mm column (BEH130 C18, 1.7 μm Waters) using solvent A (0.1% FA in LC-MS grade water) and solvent B (0.1% FA in LC-MS grade ACN) as mobile phases. The UPLC settings consisted of a sample loading flow rate of 2 μL/min for 8 min followed by a gradient elution starting with 5% of solvent B and ramping up to 35% over 220 min followed by a 10 min wash at 85% B and a 15 min equilibration step at 1% B. The flow rate for the sample run was 300 nL/min with an operating back pressure of about 3800 psi. Full scan survey spectra (m/z 375-1800) were acquired in the Orbitrap with a resolution of 30000 at m/z 400. A data dependent analysis (DDA) was employed in which the five most abundant multiply charged ions present in the survey spectrum were automatically mass-selected, fragmented by collision-induced dissociation (normalized collision energy 35%) and analysed in the LTQ. Dynamic exclusion was enabled with the exclusion list restricted to 500 entries, exclusion duration of 30 sec and mass window of 10 ppm.

MASCOT search was used to generate a list of proteins. Peptide identification was performed by searching against the SwissProt database (version 2013-2014) restricted to human entries using the Mascot search engine (v 2.5.0, Matrix Science, London, UK). The parameters included trypsin as the bdigestion enzyme with up to two missed cleavages permitted, carbamidomethyl (C) as a fixed modification and Pyroglu (N-term), Oxidation (M) and Phospho (STY) as variable modifications. Datasets were searched with a mass tolerance of ±5 ppm and a fragment mass tolerance of ±0.8 Da.

A MASCOT score cut-off of 50 was used to filter false-positive detection to a false discovery rate below 1%. PESCAL was used to obtain peak areas in extracted ion chromatograms of each identified peptide and protein abundance determined by the ratio of the sum of peptide areas of a given protein to the sum of all peptide areas. This approach for global protein quantification absolute quantification, described in 5, is similar to intensity based protein quantification (iBAQ)⁶, and total protein abundance (TPA)⁷. Proteomic data are available via the PRIDE database accession number PXD004060.

Cytokine/chemokine analysis: Cytokine and chemokines were assayed using Mesoscale Discovery Platform (MSD SI2400) according to manufacturer's instructions. Cytokine panel 1(Human) K15050D, Proinflammatory panel 1(human) K0080087, and Chemokine panel 1(Human) K0080125 were used. Samples used were lysates from the ECM enrichment protocol (described above). The amount of total protein used from each sample was between 1 and 3 μg.

Mechanical Characterisation

Flat-punch Indentation. Mechanical characterisation was performed using a previously published methodology in order to measure the modulus of the tissue samples⁸. The modulus provides a measure of the stiffness of the material that is independent of specimen geometry. Frozen tissue specimens (n=32) were fully thawed at room temperature in PBS for 1 hour before testing. Indentation was performed using an Instron ElectroPuls E1000 (Instron, UK) equipped with a 10 N load cell (resolution=0.1 mN) (Supplementary data 1a). Specimens were indented using a stainless steel plane-ended cylindrical punch with a diameter (Øᵢ) of 2 or 3 mm. Specimen thickness (Tₛ) was measured as the distance between the base of the test dish and top of the sample, each detected by applying a pre-load of 0.3-5 mN. Specimen diameter (Øₛ) was measured using callipers. In order to minimise errors in calculations of mechanical parameters, specimen to indenter ratios were Øₛ:Øᵢ≥4:1 and Tₛ: Øᵢ≤2:1⁸. Indentation was performed at room temperature with specimens fully submerged in PBS throughout testing. Tests were performed using two consecutive displacementcontrolled static loading regimes on each specimen with a recovery period of 20 min between tests. Specimens were displaced to 20% or 30% of their measured thickness at a rate of 1% .s⁻¹ followed by a displacement-hold period to allow full sample stressrelaxation, and then an unloading phase to 0% specimen strain. The resulting load detected from the sample was recorded. Green tissue dye was used to mark the surface area of tissue-indenter contact for later correlation of mechanics with tissue architecture (Supplementary data 1b). After testing, specimens were snap frozen in LN₂ and stored at −80° C. until further processing.

Mechanical quantification. Tissue modulus, E, was calculated from the obtained load displacement experimental data with the aid of a mathematical model derived from the solution of Sneddon for the axisymmetric Boussinesq problem as shown in equation 1. Full details of this model and its validation are given in a previous study by the inventors⁸

$$E = \frac{S}{2 a} ( 1 - {v}^{2} ) \qquad ( Eq . 1 )$$

The indentation stiffness, S, was calculated from the slope of the load-displacement curve defined for each tangent (Supplementary data 1c) and ‘a’ is the radius of the flat-punch indenter. Poisson's ratio, v, was assumed to be 0.5 for all samples. Mechanical values were plotted against scores determined from tissue architecture analysis.

Confocal Microscopy

Second harmonic generation. Paraffin embedded TMAs containing 3-6×1 mm tissue cores per sample were mounted in Fluoromount (Sigma, UK) and samples (n=13) were imaged via two-photon confocal microscopy to collect second harmonic generation (SHG) illumination. Images were captured on an inverted Leica laserscanning confocal TCS SP2 microscope (Leica) equipped with a tunable Ti:Sapphire femto-second multiphoton laser (Spectra-Physics). Specimens were illuminated at 820 nm and the resulting signal was collected in the backward scattering direction (epi), after filtration through a SP700 dichroic, using a photo-multiplier tube (PMT) set to collect SHG between 405-415 nm. The laser passed through a 63×1.4 NA oil immersion objective with the pinhole set to maximum resulting in a laser excitation power at the specimen of 20 mW. Specimen images were acquired with a frame average of 2 and a line average of 16 at intervals of 1 μm in the z-direction each with a field of view equal to 238.1×238.1 μm containing 1024×1024 pixels. At least three×5 μm z-stacks were collected from each individual tissue core and then analysed using Image J to measure fibre orientation.

Histochemical Analysis

Tissue architecture. Frozen tissues that were later used for RNA, matrisome and cytokine analysis were cryosectioned to 8-10 μm slices. Sections were fixed in in 4% paraformaldehyde (PFA) and stained with haematoxylin and eosin using standard methods. Tissues used in mechanical characterisation were cut in half at the centre of the tissue dye marked area and perpendicular to the direction of indentation while still frozen. Tissue was then fixed in 4% PFA for 24 h and paraffin embedded and sectioned (8 μm) using standard procedures followed by H&E staining. All tissue sections were scanned using a 3DHISTECH Panoramic 250 digital slide scanner (3DHISTECH, Hungary) and the resulting scans were analysed using Definiens software (Definiens AG, Germany). Disease scores were determined firstly by manually defining regions of interest in the tissue that represented tumour, stroma, fat (adipocytes) or other (lymphatic structure) and then training the software to recognise these regions of interest. Disease score was expressed as a percentage of the whole tissue area that contained tumour and/or stroma (FIG. 1b).

Immunohistochemical Analysis

Quantification of Immune cells, α-SMA positive cells, and adipocyte diameters. TMA cores were used for immune cell counts and quantification of α-SMA positive cells and adipocyte diameters. Paraffin embedded TMAs were heated at 60° C. for 5 min followed by 2×5 min submersion in xylene and then a series of ethanol washes of decreasing concentration for 2×2 min each (100%, 90%, 70%, and 50%). Antigen retrieval was performed for 10 min using vector antigen unmasking buffer and a pressure cooker. TMAs were then washed with DAKO wash buffer followed by application of H₂O₂ for 5 min. Blocking was performed using 5% BSA for 20 min at RT followed by incubation with primary antibody in biogenex antibody diluent for 30 min. After 3× washes, biogenex super enhancer was added for 20 min and then washed off before addition of biogenex ss label poly-HRP for 30 min. Tissue was washed three times before addition of DAB chromagen for 3 min followed by washing to stop further DAB development. TMAs were counterstained with haematoxylin followed by washing with H₂O and ethanol solutions of increasing concentration for 2 min each (50%, 70%, 90%, 100%) and then 2× xylene. Samples were then mounted and scanned using the 3DHISTECH Panoramic digital slide scanner. Immune cells were counted manually using Image J. The population of α-SMA positive cells was determined using Definiens software, firstly by setting a threshold and then quantifying the area of tissue expressing α-SMA to give a % SMA+ area. Adipocyte diameter was quantified on α-SMA stained TMAs using Panoramic Viewer software (3DHISTECH, Hungary) by measuring at least 100 adipocytes per sample (n=16) to get the population mean. For samples with tumour and stromal remodelling, adipocytes that were either in contact with stroma or totally surrounded by stroma were measured. All cell analysis was plotted versus disease score determined using Definiens software analysis of haematoxylin and eosin stained TMAs.

Matrix staining. Immunohistochemical staining for ECM proteins was performed on 4 μm slides of FFPE human omentum tissue as described above. Antibodies. The following antibodies were used for immunohistochemical analyses: anti-FOXP3 (clone 263A/E7, ab20034) from Abcam, UK; anti-CD3 (clone F7.2.38, M7254), anti-CD4 (clone 4B12, M7310), anti-CD8 (clone C8/144B, M7103), anti-CD68 (clone KP1, F7135), anti-CD45RO (clone UCHL1, M0742), anti-Ki67 (cloneMIB-1, M7240), all from Dako, UK; anti-VCAN (polyclonal, HPA004726), anti-SFRP4 (polyclonal, HPA009712), anti-COL11A1 (polyclonal, HPA052246) anti-TNC (polyclonal, HPA004823), anti-COL1A1 (polyclonal, HPA011795), anti-FN1 (polyclonal, F3648), anti-IL16 (polyclonal, HPA018467), anti-actin, α-smooth muscle (clone 1A4, A2547), all from Sigma, UK. Anti-CTSB (ab125067), and anti-COMP (ab11056), both from Abcam.

Tissue arrays. All tissues were obtained from patients with full written informed consent. Breast tissues were obtained through the Breast Cancer Campaign (now Breast Cancer Now) Tissue Bank (NRES Cambridgeshire 2 REC 10/H0308/48), and Barts Cancer Institute Breast Tissue Bank (NRES East of England 15/EE/0192). DLBCL lymph node tissues were obtained through the Local Regional Ethics Boards (05/Q0605/140). Pancreatic tissues were obtained through the City and East London REC 07/H0705/87. Tissue microarrays (TMA) were prepared from paraffin blocks with triplicate 1 mm cores taken from each biopsy material.

RNA in Situ Hybridization

Chromogenic in situ hybridization for VCAN (Probe-Hs-VCAN, Cat No. 430071, Advanced Cell Diagnostics Inc. USA) was performed using the RNAscope 2.5 HD Detection Reagent kit (Advanced Cell Diagnostics Inc.) according to the manufacturer's instructions. Briefly, 4 μm sections of FFPE human omentum samples were heated at 60° C. for 1 h before deparaffinization in two changes of xylene for 5 min, followed by two changes of 100% ethanol for 1 min. Slides were then treated with the pre-packaged hydrogen peroxide for 10 min and boiled for 15 min in the target retrieval reagent. The tissue was then dried in ethanol, outlined using a hydrophobic barrier pen and left at room temperature overnight. Slides were then incubated in the protease reagent at 40° C. in a HyBEZ Hybridization System (Advanced Cell Diagnostics Inc. USA) for 30 min, before a 2 h incubation at 40° C. with the gene-specific probe. The AMP 1-6 reagents were all subsequently hybridized at 40° C. or RT, 30 or 15 min as specified in the manufacturer's instructions. Labelled mRNAs were visualized using the included DAB reagent for 10 min, then counterstained for 2 min using 50% Gill's haematoxylin followed by 3 dips in 0.02% ammonia water. Counterstained slides were dehydrated using 70% and 95% ethanol then cleared in xylene before mounting coverslips using DPX.

PLS Regression

Model fitting. PLS regression was implemented using the R package pls (version 2.4-3)⁹. Briefly, the PLS algorithm consists of the following steps: first, the data is standardized by centering to column mean zero and scaled to unit variance (dividing columns by their standard deviation), resulting in a matrix X (genes or proteins) and vector y (disease score or tissue modulus). Second, using the linear dimension reduction t=Xw, the p predictors (genes or proteins) in X are mapped onto latent components in t. The weights w are chosen with the response y explicitly taken into account, so that the predictive performance is maximal. Next, y is regressed by ordinary least squares against the latent components t (also known as X-scores) to obtain the loadings q. Subsequently, the PLS estimate of the coefficients in y=βX+error is computed from estimates of the weight matrix w and the y-loadings via β=wq.

Prior to model fitting the data was randomly split into a “training” set of 18 samples (approximately ⅔ of data) leaving the remaining samples as a “test” set. Both training and test sets included samples ranging from low to high disease score. Using the training set a PLS model was initially fitted using 10 components with leave-oneout cross-validation. The validation results were expressed as root mean squared error of prediction (RMSEP).

$$RMSEP = \sqrt{\frac{{\sum }_{i = 1}^{n} {( {y}_{i} - {\hat{y}}_{i} )}^{2}}{n}}$$

where n is the total number of samples, yᵢ is the actual value of y (disease score or stiffness) for sample i and ŷᵢ the y-value for sample i predicted with the model under evaluation. The estimated RMSEPs were then plotted as functions of the number of components. The components that corresponded to the first local minimum RMSEP were chosen as optimal for the model. The fitted model was then used to predict the response values of the test set of samples. Since the inventors knew the true response values of the test data the inventors were able to calculate the RMSEP, which was typically very similar to the crossvalidated estimate of the training data.

Estimating confidence of model predictions and assessing the significance of model performance. In order to determine the performance of the constructed PLS models over multiple iterations of model building and testing, bootstrapping was carried out by iterating 1000 times through the whole process of random selection of training and test datasets, model fitting and recording predicted values and RMSEP. By this process, frequency distributions for the overall test accuracies (RMSEPs) and the predicted response values were obtained.

The inventors then examined the statistical significance of the performance of the constructed PLS regression models compared to random chance using permutation testing. The data was randomly shuffled across samples within each variable. This process destroyed the correlations in the data while retaining the original variance of the variables. Then the process of model building, testing prediction accuracy by RMSEP and bootstrapping was repeated using the permuted datasets. Student's t-test was then used comparing the difference in model performance over RMSEP values obtained from permutation testing and RMSEP values obtained from the original datasets to determine whether the model was statistically significant. For all models that were used throughout the study P_{realvspermuted}<2.2×10⁻¹⁶.

PLS-ranking of variables and cut-off values. The loading weights of the first component, which explained >70% of variance, were used to rank variables (genes or proteins) according to their contribution to the model ^{10,11}. Inherently this vector is calculated to maximize covariance of Xw₁ with y. To determine which variables made a significant contribution to the model, variables were removed from the model in order of weight until the bootstrapped RMSEP exceeded that of permutation testing.

Matrix Index and its Clinical Association Across Cancer Types

Based on the 22 matrisome genes, the inventors defined “matrix index” as the ratio of the mean expression of the genes positively correlated with disease score to that of the remaining negatively correlated genes. The inventors first tested the clinical association and prognostic potential of this matrix index in two large ovarian cancer datasets from the International Cancer Genome Consortium (ICGC) and The Cancer Genome Atlas (TCGA)¹², as ICGC_OV and TCGA_OV. For the ICGC_OV set, raw read counts for all annotated Ensembl genes across 93 primary tumors were extracted from the exp_seq.OV-AU.tsv.gz file in the ICGC data repository Release 20 (http://dcc.icgc.org). Only genes that achieved at least one read count per million reads (cpm) in at least ten samples were selected, with these criteria producing 18,698 filtered genes in total. After applying scale normalization, read counts were converted to log2 (cpm) using the voom function ¹³. Clinical information (e.g., overall survival (OS)) was extracted from the donor.OV-AU.tsv.gz file. For the TCGA_OV set, the normalized gene expression data profiled by Affymetrix U133a 2.0 Array and clinical data were downloaded from UCSC Cancer Browser (http://genome-cancer.ucsc.edu/), version 2015 Feb. 24. Only primary tumors were selected for further analysis, leading to 564 primary samples with both expression and OS data available.

Expression values for the matrisome genes were extracted and matrix index was calculated for each sample. For each dataset, the high and low index groups were determined using the method described previously ¹⁴. Briefly, each percentile of index between lower and upper quartiles was used in the Cox proportional hazards (Coxph) regression analysis and the best performing threshold of percentile associated with OS was determined. Survival modeling and Kaplan-Meier

(KM) analysis was undertaken using R “survival” package. OS was defined as time from diagnosis to death, or to the last follow-up date for survivors. The inventors further assessed the prognostic potential of matrix index using the multivariate analysis, accounting for age, tumor stage, grade and primary therapy outcome success. Note that for ICGC_OV set, only age and tumor stage information were available. Hazard ratio (HR) and 95% confidence interval (CI), as well as associated p-values for matrix index at the best performing threshold were derived from the Coxph regression model for both uni- and multivariate analyses.

The inventors then benchmarked the performance of matrix index in prognostics against other existing ovarian cancer signatures (including the 193-gene signature from TCGA) and other relevant stroma and immune signatures extracted from literature on the TCGA_OV set. For expression-based signatures, firstly consensus clustering, using ConsensusClusterPlus R package¹⁵, was performed based on normalized expression values to split patients. After sample grouping, both uni and multivariate survival analyses with OS were subsequently conducted using the Coxph regression. The prognostic value for the matrisome genes solely based on expression clustering was also assessed in this way.

The inventors further expanded the survival analysis of matrix index into other cancer types and datasets, including additional 33 TCGA cancer sets and 2 ICGC sets (Supplementary Table 2). For these TCGA sets, the gene expression Illumina HiSegV2 RNA-seq normalized data were used, available from UCSC Cancer Browser. For the ICGC chronic lymphocytic leukemia dataset, ICGC_CLLE-ES, the expression array data was used. The two pancreatic cancer sets, ICGC_PACA-AU and Stratford_PDAC, were based on data previously described ¹⁶. In total, the inventors assessed the prognostic values of matrix index in 38 cancer sets including the two ovarian sets. Six datasets were further excluded from the results due to the large HR 95% CI, resulting in final 32 valid datasets (Supplementary Table 2). The same survival analysis protocol was applied for each dataset as above. For those datasets, pathogenic T-stage was used when tumor grade information was unavailable, and target molecular therapy or radiation therapy (in the “yes” or “no” category) was used if primary therapy outcome success information was not available.

Additional Information on Statistical Analyses

All graphics and statistical analyses were performed in the statistical programming language R (version 3.1.3). For PLS regression models, a fourth square root transformation was applied to the proteomics and biomechanical data. Univariate correlations were calculated using spearman's correlation or pearson's correlation applied on linear, log or square-root transformed data. Overrepresented Gene Ontology annotations from the differentially expressed genes were identified by a modified Fisher's exact test using the web-based tool PANTHER (version 10) ¹⁷. Enrichment p-values were calculated with a modified Fishers exact test and Bonferroni multiple testing correction.

Results

Study Design

The inventors measured the biomechanics, tissue architecture and cellularity in omental biopsies from 36 HGSOC patients and integrated these with RNA and protein data from the same samples (FIG. 1A). To represent disease progression, samples included uninvolved omentum, biopsies adjacent to tumor islands and heavily diseased tissue. Tissue architecture was measured as ‘disease score’ by digital histopathology. As remodelling of the omentum was extensive even though the malignant cell areas comprised a minor proportion of the tissue, the inventors defined the disease score as the percentage of tissue area occupied by malignant cells and stroma (FIG. 1B).

After alignment and filtering, RNA sequencing identified 15,441 protein-coding genes. For proteomic analysis of the same biopsies the inventors focused on the ECM using a modification of a method that enriches for the matrisome ¹⁸ detecting 145 ECM-associated proteins. Twenty-nine cytokine and chemokines were measured using an electro-chemiluminescence assay. The inventors then used a multivariate regression method—partial least squares (PLS) ^{19,20}—to model the relationship between molecular components and the higher-order features. PLS model weights were used to rank genes and proteins according to their influence on the model, and a permutation-derived threshold was applied to determine those that were most strongly associated with stiffness, disease score or cellularity (FIG. 10) ^{21,22}.

Tissue Modulus (Stiffness), Disease Progression, Protein and Gene Profiles

As increased stiffness has been linked with tumor progression ^{23,24}, the inventors used a mechanical indentation methodology ²⁵ to determine tissue modulus (which describes material stiffness independent of sample histology) and viscoelastic stress-relaxation properties of the samples, and measured disease score from histological sections of the tested area (FIG. 2A, FIG. 13). Biopsies with a high disease score displayed a non-linear loading response and greater stress relaxation while there was a relatively linear loading response in low disease score tissue (FIG. 2B, FIG. 13C). Tissue modulus in high disease score biopsies was also one-two orders of magnitude higher than in low disease biopsies. There were significant positive correlations between tissue modulus and malignant cell area, the stromal area and the two combined (i.e. disease score) (FIG. 2C, FIG. 13D). Tissue modulus in high disease score biopsies increased by one-two orders of magnitude compared to low disease biopsies. The inventors concluded that tissue stiffness is associated with disease progression.

Using the PLS method, the inventors identified 64 ECM-associated proteins, mainly glycoproteins, that accurately predicted tissue modulus (r²=0.69) (FIG. 2D, FIG. 14A). There were also 405 genes that predicted tissue modulus (FIG. 2E, FIG. 14B) of which 38 also featured as proteins in FIG. 2D. The data show that tissue modulus was determined by a subset of ECM-associated genes and proteins.

The inventors also modeled tissue modulus against the entire transcriptome (FIG. 14C). Genes associated with cell metabolism, cell communication, wound healing, ECM organization, as well as development, correlated with tissue modulus (FIG. 14D). FIG. 2F shows the PLS prediction plot and the top 50 genes from this signature.

Identification of ECM Proteins and Gene Signatures that Explain Disease Score

The inventors next studied how ECM proteins and genes changed with increasing disease score. In terms of relative mass ratios, the major matrix proteins in the six samples with the lowest disease score were collagen 1, 6 and 3, the glycoprotein fibrillin, the ECM regulator alpha-2-macroprotein, and the basement membrane proteoglycans lumican and heparin sulphate proteoglycan-2. The 10 biopsies with the highest disease score had significant reductions in collagen 1, an expansion of ECM-glycoproteins fibrinogen and fibronectin, as well as increases in proteoglycans, secreted factors, and affiliated proteins, (FDR<0.1) (FIG. 3A). Extending the analysis to the entire sample set the inventors found that as disease score increased levels of some ECM-associated proteins decreased and others increased. Comparing the relative mass ratio of all ECM-associated proteins with disease score, the inventors found that 18 proteins decreased and 49 proteins increased with disease progression (FIG. 3B). Of these, 58 proteins ranked top in PLS modeling of disease score (r²=0.70), defining an ECM signature of disease score (FIG. 3C).

412 of the 764 matrisome genes also predicted disease score; the top 60 are shown in FIG. 3D with 27 ECM-associated molecules predicting disease score at both the gene and protein level (FIG. 3E, FIG. 15A). The inventors used IHC to detect four of these proteins in HGSOC omentum detecting all four within stromal regions (FIG. 3F). As collagen organisation strongly influences both tissue mechanics and cell behavior ^{26,27} and collagen composition changed with disease score and tissue modulus, the inventors utilised two-photon microscopy to visualise collagen fibres using second harmonic generation (SHG) label-free illumination (FIG. 3G). In low disease score tissues collagen fibres were thin and arranged mostly around the adipocytes. In high disease score tissues, there were dense arrays of long collagen bundles with an apparent micro-scale orientation preference. Collagen orientation correlated strongly with disease score.

These experiments demonstrated dynamic changes of matrisome proteins and genes during development of HGSOC metastases and show, for the first time, the complexity of the matrix evolution during development of metastases. Changes in disease score could also be modelled in the entire transcriptome dataset. As expected there was a strong overlap with disease score-associated genes and proteins (74% and 75% respectively) and those were significantly associated with tissue modulus. As with tissue modulus, biological processes associated with disease score included cell metabolism, adhesion, communication, and ECM organization but immune response pathways also featured significantly (FIG. 15B).

Changes in Cellularity with Disease Progression and Correlation with Tissue Modulus

Using a tissue microarray constructed from the biopsies the inventors quantified the major non-malignant cellular components, adipocytes, fibroblasts and leukocytes. The area occupied by adipocytes decreased with disease score and there were negative correlations between disease score, adipocyte diameter and levels of the adipogenic transcription factor PPARγ mRNA (FIG. 4A). This may reflect research showing that adipocytes can provide energy for ovarian cancer cell growth ¹⁴. Using α-SMA as a marker of cancer-associated fibroblasts ²⁸ the inventors assessed the area of the tissue occupied by α-SMA+ cells and found a strong positive correlation with disease score (FIG. 4B).

The inventors then correlated densities of six major leukocyte subtypes against disease score. In all cases a highly significant positive correlation was seen between leukocyte density and disease score (p<0.001) (FIG. 4C, FIG. 16A). These cell densities also significantly correlated with their corresponding immune gene expression signatures extracted from the RNAseq data. Densities of T cells with surface markers CD3, CD4, CD8 and CD45RO strongly correlated with each other (p<0.001, r>0.6) but CD68+ macrophage density only weakly correlated with the other leukocytes (p<0.05, r<0.5) (FIG. 4D). Finally the inventors looked for correlations between cellularity and tissue modulus. α-SMA+ cells showed the strongest correlation (FIG. 16B). Associations between increasing leukocyte density and the tissue modulus were not as striking, although there was weak significance with Treg density.

Therefore, as metastases developed in the omentum, the fatty tissue was replaced by fibroblasts, lymphocytes and macrophages even in the presence of very small malignant cell deposits.

Cytokine and Chemokine Networks in the TME

As cytokine networks are major determinants of leukocyte density and phenotype in the TME ^{3,29,30}, the inventors asked if the cytokine proteins and genes the inventors detected could inform them about the networks that regulate omental metastases. The inventors constructed heatmaps showing pairwise comparisons of cytokine protein and gene transcription levels (FIG. 4E, FIG. 16C). Overall the protein gene correlation was 30%, in line with other studies ^{31,32}. The heatmaps show five significant co-expressions at both gene and protein level: IL6 with IL1A, IL1B, and IL8, CSF2 with IL8, and CCL4 with CCL3. IL6 was of particular interest as the inventors previously identified this as a major mediator of cytokine networks in ovarian cancer ^{29,33}.

To understand how these mediators may influence immune cells in the TME, the inventors correlated leukocyte density against cytokine protein levels. There were eight significant correlations (FIG. 4F), the strongest of which was the association between IL16, a chemoattractant and modulator of T cell function ³⁴, and CD3, CD45RO and CD8 cell density. These correlations became stronger with the 10 samples with the highest disease score (FIG. 16D). IHC revealed IL16 protein in both malignant and stromal areas, with a higher density in the former (FIG. 4G). There was also a high correlation between overall cell proliferation assessed by Ki67 and LTA, IL17A, IL15, CXCL10. Finally the inventors asked if levels of any of the cytokines and chemokines associated with disease score and/or tissue modulus. While none of the correlations were as significant as for ECM proteins and genes, there were weak but significant associations with disease score and/or tissue modulus with IL12B, IL16, VEGF, TNF, CCLs 3,4,11,17,26, and CXCL10.

These results suggest that malignant cell-derived cytokine and chemokine networks in the omental metastases regulate leukocyte density and overall proliferative index. Unexpectedly, the inventors identified the CD4 ligand IL16 as a potential major mediator of the leukocyte infiltrate. Increased tissue and serum levels of IL16 have been reported during tumor development in laying hen models of ovarian cancer and in a small cohort of ovarian cancer patients ³⁵.

ECM-Associated Gene Expression Patterns and the ‘Matrix Index’

At this stage of the project, the multi-level analysis of the TME had given the inventors novel insights into the evolution and regulation of a TME and generated a resource for developing and validating complex in vitro TME models. However, the in-depth study had focused on just one metastatic site of one human cancer. Did the results have any relationship to primary ovarian cancer or other cancers? As matrix remodeling is a common feature of many human cancers and the matrisome changes were strong predictors of disease score and tissue modulus, the inventors decided to investigate the wider significance of the ECM changes. The inventors determined the smallest number of ECM-associated genes and proteins that defined disease score and tissue modulus in the sample set. 341 genes and 53 proteins (FIG. 5A, Supplementary Table 1) correlated significantly with tissue modulus and disease score. Twenty-two molecules were common to all of the analyses with a gene:protein concordance of 68% (FIG. 5A, FIG. 17A). Thirteen of the 22 proteins had documented protein:protein interactions (FIG. 5B).

The inventors then calculated a ‘matrix index’: the ratio between the mean expression levels of the six positively regulated genes and the mean expression levels of the sixteen negatively regulated genes. The matrix index of each sample significantly correlated with disease score and tissue modulus (p<0.0001) (FIG. 5C). There were also significant positive and negative correlations between matrix index and immune cell signatures in the corresponding RNAseq data (FIG. 5D), notably Treg and Th2 cell signatures; cell subtypes associated with tumor promotion and immune suppression e.g. ³⁶. There was also a modest statistically significant relationship between disease score and entropy as a measure of clonal abundance for T and B cells. This suggests there may be specific expanded populations of cells.

Relevance of Matrix Index to other Stages of HGSOC and Prognosis

As the matrix index positively correlated with disease score, tissue modulus and some immune suppressive signatures in the sample set, the inventors wondered if it would distinguish ovarian cancer patients with a poorer prognosis in untreated primary tumors. The inventors extracted expression values from two publicly available HGSOC gene expression datasets and calculated the matrix index for each sample. The high and low index groups were determined using a method described previously ³⁷. High matrix index significantly correlated with shorter overall HGSOC patient survival in both the ICGC and TCGA gene expression datasets, as well as in the original sample set (FIG. 5E, FIG. 17B-D).

Using TCGA ovarian cancer dataset, the inventors next evaluated the power of the matrix index against nine other prognostic gene expression signatures in ovarian and other cancers, including signatures for stromal and immune responses ³⁸⁻⁴⁶. In terms of hazard-ratio scores, matrix index was in the top three after the 26-gene breast cancer stromal signature reported by Finak et al ⁴⁶ and the 193-transcriptional signature from TCGA ¹⁰ (FIG. 5F, left panel). However, using multivariate analysis, matrix index was the single significant predictor of ovarian cancer survival independently of age, stage, grade and treatment outcome (FIG. 5F, right panel).

Matrix Index in other Human Cancers

The inventors then calculated matrix index values in 30 other publicly available gene expression datasets from epithelial, mesenchymal and haematologic malignancies analysing data from 9215 human cancer biopsies including the HGSOC samples. High matrix index was an indicator of poor prognosis in epithelial and mesenchymal cancers but not in haematological cancers, melanoma and glioblastoma (FIG. 6A and FIG. 18A). Using univariate analysis, high matrix index predicted shorter overall patient survival in 15 datasets representing 13 major cancer types (p<0.05) (FIG. 18B, Supplementary Table 2). The range of matrix index values across all these cancers databases had a median value close to 1.0 (FIG. 18C). The inventors believe this provides further evidence that the pattern of ECM-associated gene expression determined by the matrix index may be a common feature of some human cancers. Remarkably, multivariate analysis showed that the prognostic value of the matrix index was independent of age, stage, grade and response to primary treatment in 15 of the datasets representing 13 major cancer types (p<0.05) (FIG. 6B).

Using IHC, the inventors confirmed the presence of four of the upregulated matrix index proteins FN1, COL11A1, CTSB, and COMP, in three tissue microarrays from triple negative breast cancer (TNBC), pancreatic ductal adenocarcinoma (PDAC), and diffuse large B-cell lymphoma (DLBCL) (FIG. 6C). These cancers reflected the range of hazard ratios for high matrix index in FIG. 6B. Digital microscopy analysis showed the highest staining level in TNBC (FIG. 6D), in keeping with the matrix index score for this cancer (FIG. 18C). FN1, COMP, and CTSB were present in stroma and fibroblastic cells of all tumors. COL11A1 was located within the malignant cells in all biopsies. FN1 was also found in malignant PDAC cells and in immune cells in DLBCL. CTSB was located in macrophages in TNBC and PDAC, and tumor cells in DLBCL.

Data Resource

All data in this paper will be provided in a mine-able web-based resource http://www.canbuild.org.uk currently under construction. Users will be able to download, visualize, analyse and integrate across datasets.

Conclusions

The inventors conclude that using multi-component analysis of samples from an evolving metastatic site of one human cancer type has relevance to other cancer types and stages. Focusing on ECM-associated molecules, the inventors identified a pattern of matrix gene expression that suggests a common matrix response in human cancer. The data also show that that multi-level study of cancer biopsies can complement larger ‘omic’ molecular cancer datasets.

While it is now accepted that malignant cell clones undergo complex Darwinian evolution, the microenvironment generated by malignant cells may be more consistent. It is already known that high lymphocyte density is a common indicator of good prognosis at different stages of disease in many malignancies including HGSOC ^{16,47}. The inventors suggest that another common feature of TMEs may be patterns of ECM-associated proteins and that these may also have prognostic significance.

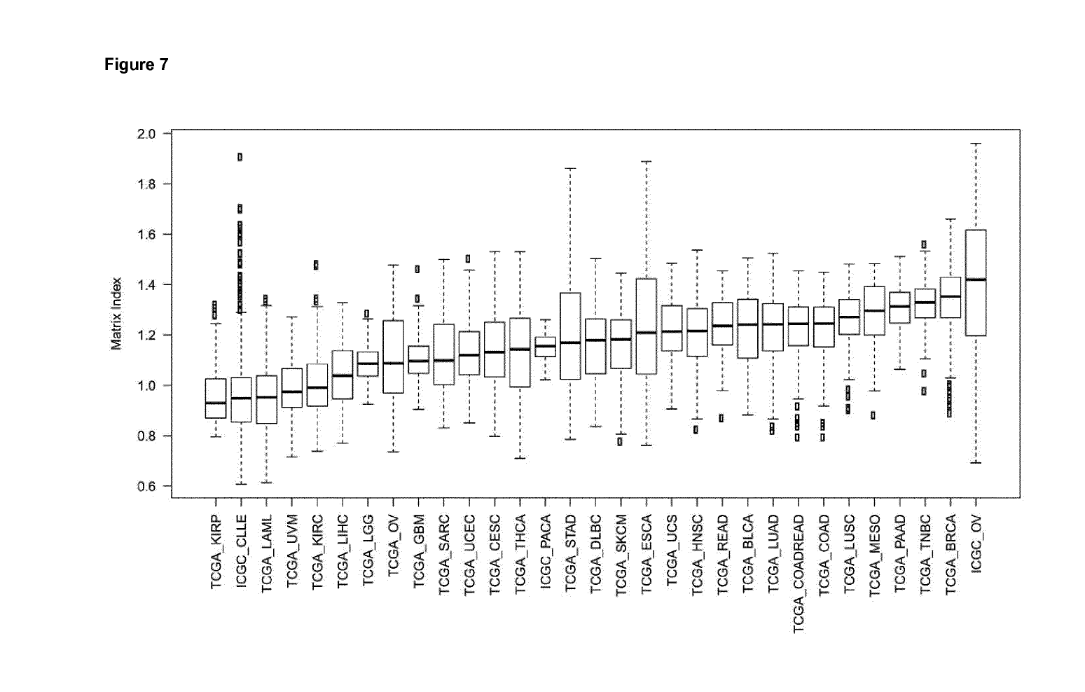

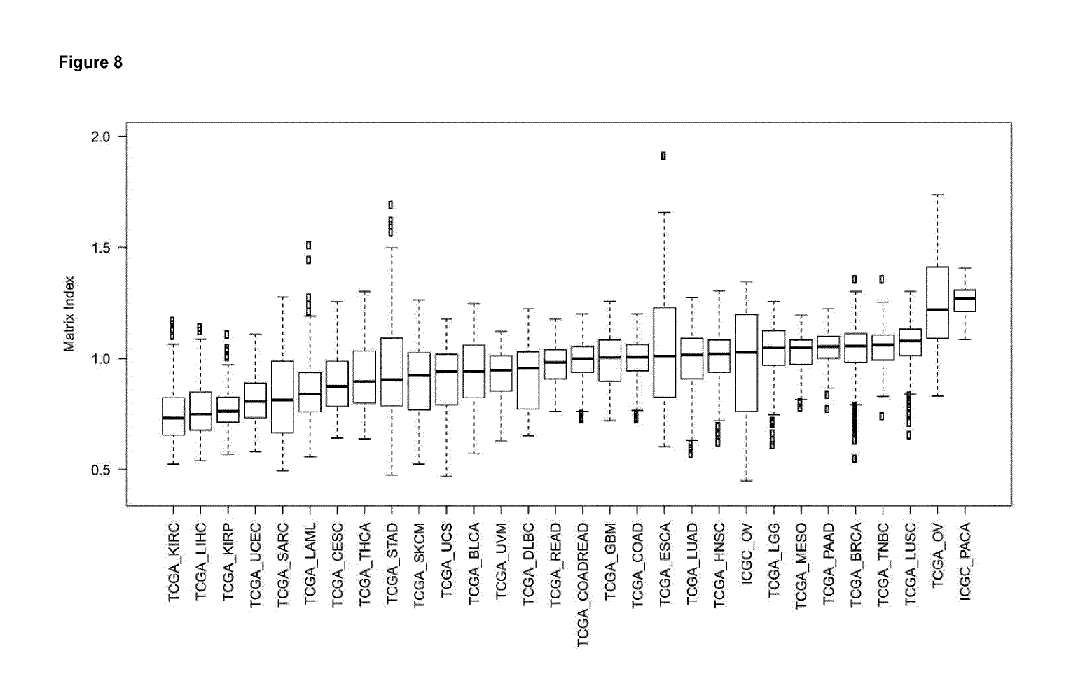

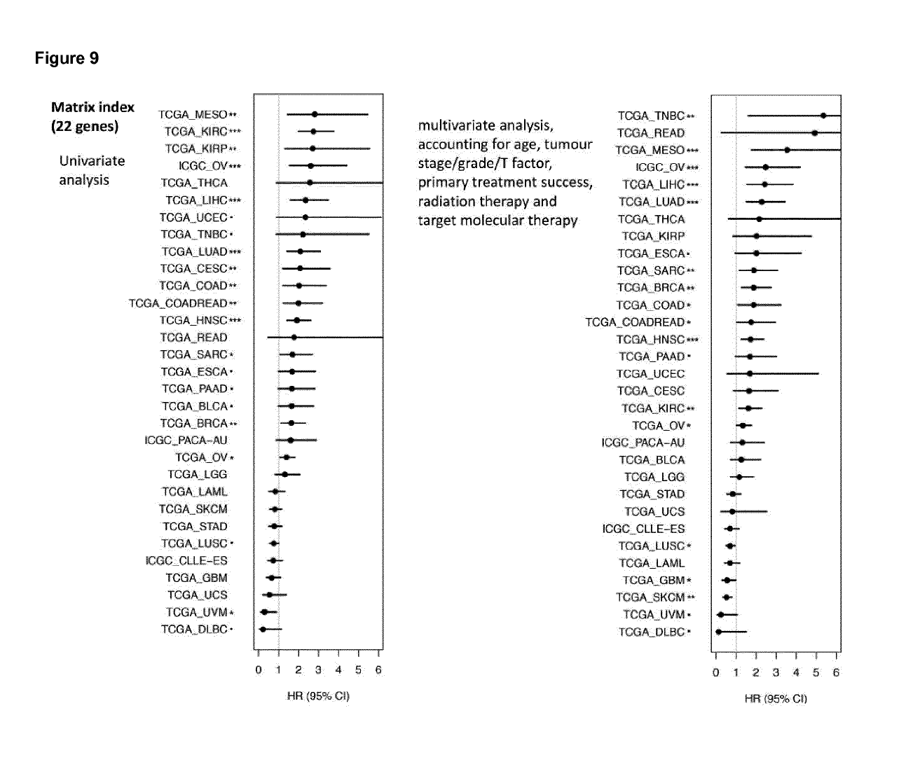

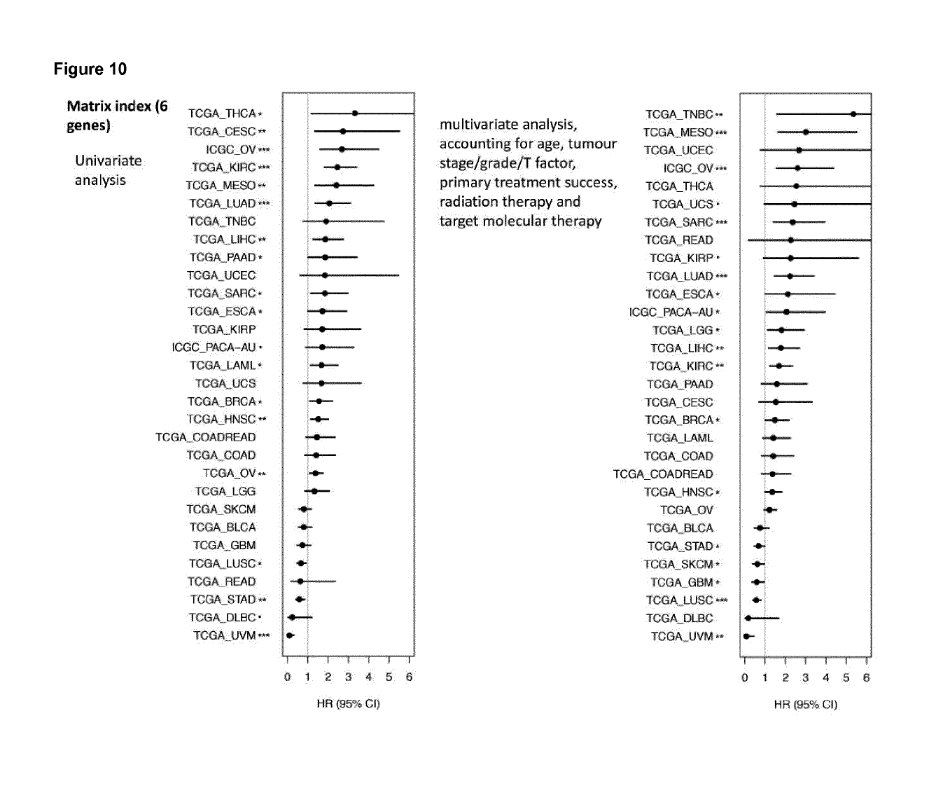

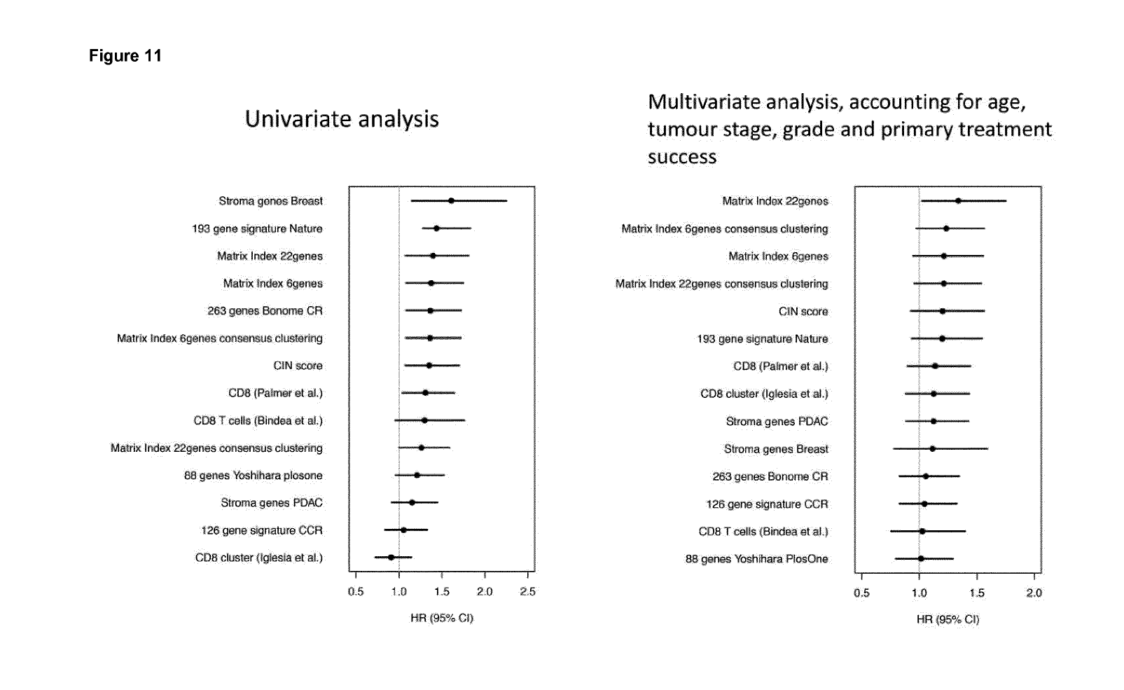

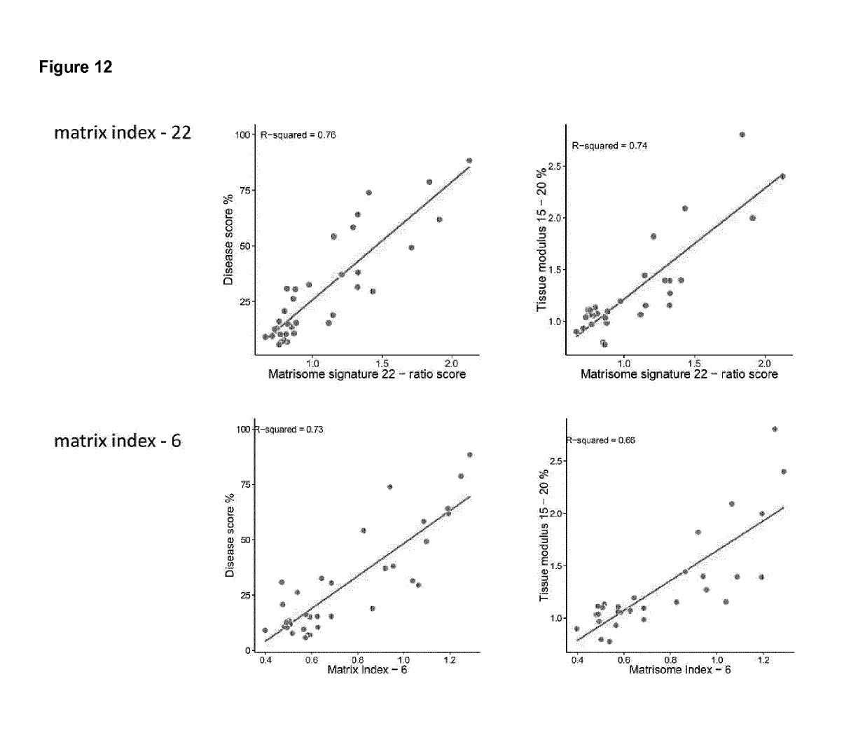

Within the 22 matrix index genes, 6 gene clusters with highly correlative expression profiles were identified using consensus clustering. From each cluster the gene with highest correlation to disease score was selected as a representative of the cluster. The resulting 6-gene matrix index retained correlation with disease score and tissue modulus and was prognostic in: mesothelioma, ovarian cancer, uterine carcinoma, sarcoma, rectum adenocarcinoma, kidney papillary cell carcinoma, lung adenocarcinoma, esophageal carcinoma, pancreatic adenocarcinoma, brain lower grade glioma, liver hepatocellular carcinoma, kidney clear cell carcinoma, breast invasive carcinoma, head and neck squamous cell carcinoma, stomach adenocarcinoma, skin cutaneous melanoma, glioblastoma multiforme, lung squamous cell carcinoma, uveal melanoma. The six up regulated genes that were most significantly related to disease score and tissue modulus in the analysis are COL11A1 , COMP, VCAN, FN1, COL1A1 and CTSB. The effectiveness of the 22 matrix index genes and the 6 matrix index genes in predicting cancer outcome is shown in FIGS. 7 to 12. Note the ability both panels to predict outcome in a range of cancers, including when benchmarked against other prognostic signatures (FIG. 11). FIG. 12 shows a direct comparison between the 6 gene index and the 22 gene index and notes that the 6 gene index significantly correlates with disease score and tissue modulus and is close to the 22 gene index.

But why does an index of ECM-associated gene expression define patients with poor prognosis in multiple human cancers? The study found a strong association between α-SMA density, disease score and tissue modulus and there are several examples in the literature of poor prognostic fibroblast, desmoplastic, wound healing and stromal signatures in individual cancer types e.g. 43,46. However, the signature the inventors have identified is distinct from the ECM molecules described in the above research and is common to thirteen different cancers. Malignant cell response to tumor-associated fibrosis, and the stromal cell phenotypes that contribute to ECM deposition, can vary within and between major cancer types. This was shown in great detail recently in a study of experimental and human pancreatic cancers where a distinct malignant cell genotype modulated the fibrotic phenotype of the tissue and pathology 9. This does not argue against the finding of the inventors because the inventors have found the matrix index is variable between different cases of each cancer. The reason why the inventors have identified a pattern of ECM-associated molecules that has prognostic significance to many different cancer types may be because the inventors have taken a different approach to other studies. The inventors have used metastatic samples with a range of disease involvement, the inventors have analysed the entire matrisome of the tissue and then related this to higher-order features—extent of disease and stiffness.

As the predictive power of the matrix index was independent of age, stage and response to primary treatment, the inventors suggest that the pattern of change in ECM proteins may reflect increased propensity of the malignant cells to establish metastases. Another explanation for the association with poor prognosis could be that this configuration of ECM molecules prevents infiltration of host anti-tumor immune cells.

If the inventors have identified a common and especially detrimental signature of tumor-associated fibrosis then agents that could reconfigure the cancer ECM could have wide applicability in solid cancers and may enhance the action of immunotherapies, especially given the association of high matrix index with immunosuppressive T cell signatures.

Acknowledgements

This project was funded by the European Research Council (ERC322566) and Cancer Research UK (A16354,A13034,A19694). The inventors thank Barts Trust Oncology Surgeons for sample provision and Prof. Kairbaan Hodivala-Dilke for useful discussion. The inventors also thank Andrew Clear, Dr Joanne ChinAleong, Dr Prabhu Arumugam and Dr Sally Dreger for technical help with the tissue microarrays, George Elia and the BCI Pathology Core, Christof Smith and Dr Dante Bortone for help with bioinformatics analysis of the immune cell signatures and Dr Jackie McDermott for histopathological analysis of the TMA samples. Finally the inventors express their gratitude to the patients for donating the samples without which this work would not have been possible.

Supp table 1

Supp table 1 cont.

Supp table 1 cont

Supp table 1 cont

Supp table 2

Supp table 2 cont

### References for Materials and Methods

1. Li, B. & Dewey, C. N. RSEM: accurate transcript quantification from RNASeq data with or without a reference genome. BMC Bioinformatics 12, 323, doi:10.1186/1471-2105-12-323 (2011).

2. Langmead, B., Trapnell, C., Pop, M. & Salzberg, S. L. Ultrafast and memoryefficient alignment of short DNA sequences to the human genome. Genome Biol 10, R25, doi:10.1186/gb-2009-10-3-r25 (2009).

3. Robinson, M. D., McCarthy, D. J. & Smyth, G. K. edgeR: a Bioconductor package for differential expression analysis of digital gene expression data. Bioinformatics 26, 139-140, doi:10.1093/bioinformatics/btp616 (2010).

4. Naba, A. et al. The matrisome: in silico definition and in vivo characterization by proteomics of normal and tumor extracellular matrices. Mol Cell Proteomics 11, M111 014647, doi:10.1074/mcp.M111.014647 (2012).

5. Cutillas, P. R. & Vanhaesebroeck, B. Quantitative profile of five murine core proteomes using label-free functional proteomics. Mol Cell Proteomics 6, 1560-1573, doi:10.1074/mcp.M700037-MCP200 (2007).

6. Schwanhausser, B. et al. Global quantification of mammalian gene expression control. Nature 473, 337-342, doi:10.1038/nature10098 (2011).

7. Wisniewski, J. R. et al. Extensive quantitative remodeling of the proteome between normal colon tissue and adenocarcinoma. Mol Syst Biol 8, 611, doi:10.1038/msb.2012.44 (2012).

8. Delaine-Smith, R. M., Burney, S., Balkwill, F. R. & Knight, M. M. Experimental validation of a flat punch indentation methodology calibrated against unconfined compression tests for determination of soft tissue biomechanics. J Mech Behav Biomed Mater 60, 401-415, doi:10.1016/j.jmbbm.2016.02.019 (2016).

9. Mevik, B. H. & Wehrens, R. The pls package: Principal component and partial least squares regression in R. Journal of Statistical Software 18, 1-23 (2007).

10. Mehmood, T., Liland, K. H., Snipen, L. & Saebo, S. A review of variable selection methods in Partial Least Squares Regression. Chemometrics and Intelligent Laboratory Systems 118, 62-69, doi:10.1016/j.chemolab.2012.07.010 (2012).

11. Johansson, D., Lindgren, P. & Berglund, A. A multivariate approach applied to microarray data for identification of genes with cell cycle-coupled transcription. Bioinformatics 19, 467-473 (2003).

12. Integrated genomic analyses of ovarian carcinoma. Nature 474, 609-615, doi:10.1038/nature10166 (2011).

13. Law, C. W., Chen, Y., Shi, W. & Smyth, G. K. voom: Precision weights unlock linear model analysis tools for RNA-seq read counts. Genome Biol 15, R29, doi:10.1186/gb-2014-15-2-r29 (2014).

14. Mihaly, Z. et al. A meta-analysis of gene expression-based biomarkers predicting outcome after tamoxifen treatment in breast cancer. Breast Cancer Res Treat 140, 219-232, doi:10.1007/s10549-013-2622-y (2013).

15. Wilkerson, M. D. & Hayes, D. N. ConsensusClusterPlus: a class discovery tool with confidence assessments and item tracking. Bioinformatics 26, 1572-1573, doi:10.1093/bioinformatics/btq170 (2010).

16. Haider, S. et al. A multi-gene signature predicts outcome in patients with pancreatic ductal adenocarcinoma. Genome Med 6, 105, doi:10.1186/s13073-014-0105-3 (2014).

17. Mi, H., Muruganujan, A. & Thomas, P. D. PANTHER in 2013: modeling the evolution of gene function, and other gene attributes, in the context of phylogenetic trees. Nucleic Acids Res 41, D377-386, doi:10.1093/nar/gks1118 (2013).

18. Naba, A. et al. The matrisome: in silico definition and in vivo characterization by proteomics of normal and tumor extracellular matrices. Molecular & cellular proteomics:MCP 11, M111 014647, doi:10.1074/mcp.M111.014647 (2012).

19. Wold, S., Ruhe, A., Wold, H. and Dunn, III, W. J. The collinearity problem in linear regression. the partial least squares approach to generalized inverses. SIAM J. Sci. Stat. Comput. 5, 735-743 (1984).

20. Wold, H. in In Multivariate Analysis (Academic press, New York, 1966).

21. Johansson, D., Lindgren, P. & Berglund, A. A multivariate approach applied to microarray data for identification of genes with cell cycle-coupled transcription. Bioinformatics 19, 467-473 (2003).

22. Mehmood, T., Liland, K. H., Snipen, L. & Saebo, S. A review of variable selection methods in Partial Least Squares Regression. Chemometr Intell Lab 118, 62-69, doi:10.1016/j.chemolab.2012.07.010 (2012).

23. Krouskop, T. A., Wheeler, T. M., Kallel, F., Garra, B. S. & Hall, T. Elastic moduli of breast and prostate tissues under compression. Ultrason Imaging 20, 260-274 (1998).

24. Levental, K. R. et al. Matrix crosslinking forces tumor progression by enhancing integrin signaling. Cell 139, 891-906, doi:S0092-8674(09)01353-1 [pii]10.1016/j.cell.2009.10.027 (2009).

25. Delaine-Smith, R. M., Burney, S., Balkwill, F. R. & Knight, M. M. Experimental validation of a flat punch indentation methodology calibrated against unconfined compression tests for determination of soft tissue biomechanics. J Mech Behav Biomed Mater 60, 401-415, doi:10.1016/j.jmbbm.2016.02.019 (2016).

26. Trappmann, B. et al. Extracellular-matrix tethering regulates stem-cell fate. Nat Mater 11, 642-649, doi:10.1038/nmat3339 (2012).

27. Delaine-Smith, R. M., Green, N. H., Matcher, S. J., MacNeil, S. & Reilly, G. C. Monitoring fibrous scaffold guidance of three-dimensional collagen organisation using minimally-invasive second harmonic generation. PLoS One 9, e89761, doi:10.1371/journal.pone.0089761 (2014).

28. Kalluri, R. & Zeisberg, M. Fibroblasts in cancer. Nat Rev Cancer 6, 392-401, doi:10.1038/nrc1877 (2006).

29. Kulbe, H. et al. A Dynamic Inflammatory Cytokine Network in the Human Ovarian Cancer Microenvironment. Cancer research 72, 66-75, doi:10.1158/0008-5472.CAN- 11-2178 (2012).

30. Allavena, P., Germano, G., Marchesi, F. & Mantovani, A. Chemokines in cancer related inflammation. Exp Cell Res 317, 664-673, doi:10.1016/j.yexcr.2010.11.013 (2011).

31. Vogel, C. & Marcotte, E. M. Insights into the regulation of protein abundance from proteomic and transcriptomic analyses. Nat Rev Genet 13, 227-232, doi:10.1038/nrg3185 (2012).

32. Koussounadis, A., Langdon, S. P., Um, I. H., Harrison, D. J. & Smith, V. A. Relationship between differentially expressed mRNA and mRNA-protein correlations in a xenograft model system. Sci Rep 5, 10775, doi:10.1038/srep10775 (2015).

33. Coward, J. et al. Interleukin-6 as a Therapeutic Target in Human Ovarian Cancer. Clinical cancer research: an official journal of the American Association for Cancer Research 17, 6083-6096, doi:10.1158/1078-0432.CCR-11-0945 (2011).

34. Cruikshank, W. W., Kornfeld, H. & Center, D. M. Interleukin-16. J Leukoc Biol 67, 757-766 (2000).

35. Yellapa, A. et al. Interleukin 16 expression changes in association with ovarian malignant transformation. Am J Obstet Gynecol 210, 272 e271-210, doi:10.1016/j.ajog.2013.12.041 (2014).

36. Singh, M., Loftus, T., Webb, E. & Benencia, F. Minireview: Regulatory T Cells and Ovarian Cancer. Immunol Invest, 1-9, doi:10.1080/08820139.2016.1186689 (2016).

37. Mihaly, Z. et al. A meta-analysis of gene expression-based biomarkers predicting outcome after tamoxifen treatment in breast cancer. Breast cancer research and treatment 140, 219-232, doi:10.1007/s10549-013-2622-y (2013).

38. Bonome, T. et al. A gene signature predicting for survival in suboptimally debulked patients with overian cancer. Cancer Res 68, 5478-5486 (2008).

39. Cancer Genome Atlas Research, N. Comprehensive genomic characterization of squamous cell lung cancers. Nature 489, 519-525, doi:10.1038/nature11404 (2012).

40. Palmer, C., Diehn, M., Alizadeh, A. A. & Brown, P. O. Cell-type specific gene expression profiles of leukocytes in human peripheral blood. BMC Genomics 7, 115, doi:10.1186/1471-2164-7-115 (2006).

41. Bindea, G. et al. Spatiotemporal dynamics of intratumoral immune cells reveal the immune landscape in human cancer. Immunity 39, 782-795, doi:10.1016/j.immuni.2013.10.003 (2013).

42. Yoshihara, K. et al. Gene expression profile for predicting survival in advanced-stage serous ovarian cancer across two independent datasets. PLoS One 5, e9615, doi:10.1371/journal.pone.0009615 (2010).

43. Moffitt, R. A. et al. Virtual microdissection identifies distinct tumor- and stromaspecific subtypes of pancreatic ductal adenocarcinoma. Nat Genet 47, 1168-1178, doi:10.1038/ng.3398 (2015).

44. Iglesia, M. D. et al. Prognostic B-cell signatures using mRNA-seq in patients with subtype-specific breast and ovarian cancer. Clin Cancer Res 20, 3818-3829, doi:10.1158/1078-0432.CCR-13-3368 (2014).

45. Yoshihara, K. et al. High-risk ovarian cancer based on 126-gene expression signature is uniquely characterized by downregulation of antigen presentation pathway. Clin Cancer Res 18, 1374-1385, doi:10.1158/1078-0432.CCR-11-2725 (2012).

46. Finak, G. et al. Stromel gene expression predicts clinical outcome in breast cancer. Nat Med 14, 518-527, doi:10.1038/nm1764 (2008).

47. Mlecnik, B. et al. The tumor microenvironment and Immunoscore are critical determinants of dissemination to distant metastasis. Sci Transl Med 8, 327ra326, doi:10.1126/scitranslmed.aad6352 (2016).

48. Böhm S, Montfort A, Pearce O M T, Topping J, Chakravarty P, Everitt GLA, Clear A, McDermott JR, Ennis D, Dowe T, Fitzpatrick A, Brockbank E C, Lawrence A C, Jeyarajah A, Faruqi A Z, McNeish I A, Singh N, Lockley M, Balkwill F R. Neoadjuvant chemotherapy modulates the immune microenvironment in metastases of tubo-ovarian high-grade serous carcinoma. Clinical Cancer Research. 2016 Jun. 15 22; 3025. doi: 10.1158/1078-0432.CCR-15-2657

1-89. (canceled)

90. A method of diagnosing or prognosing cancer, comprising measuring, in a patient sample, the expression or level of at least two genes or gene expression products selected from the group consisting of COL11A1, CTSB, ANXA6, LGALS3, ANXA1, AB13BP, COMP, COL1A1, LAMB1, CTSG, LAMA4, TNXB, FN1, AGT, FBLN2, HSPG2, COL6A6, VCAN, ANXA5, LAMC1, COL15A1 and VWF.

91. The method of claim 90, wherein the method comprises measuring the expression of at least one gene selected from the group consisting of COL11A1, COMP, FN1, VCAN, CTSB and COL1A1 and at least one gene selected from the group consisting of ANXA6, LGALS3, ANXA1, AB13BP, LAMB1, CTSG, LAMA4, TNXB, AGT, FBLN2, HSPG2, COL6A6, ANXA5, LAMC1, COL15A1 and VWF.

92. The method of claim 90, wherein the method comprises measuring the expression of CTSB and LAMC1.

93. The method of 90, wherein the method comprises measuring the expression of:
(i) CTSB;
(ii) at least gene selected from the group consisting of COL11A1, COMP, FN1, VCAN and COL1A1;
(iii) at least gene selected from the group consisting of ANXA6, LGALS3 and AGT;
(iv) at least gene selected from the group consisting of LAMA4, COL6A6, AB13BP, TNXB, LAMB1 and CTSG;
(v) LAMC1; and
(vi) at least gene selected from the group consisting of HSPG2, ANXA5, ANXA1, FBLN2, COL15A1 and VWF.

94. The method of claim 90, wherein the method comprises measuring the expression of COL11A1, ANXA6, LAMC1, CTSB, LAMA4 and HSPG2.

95. The method of claim 90, wherein the method comprises contacting the sample with a binding molecule or binding molecules specific for the at least two genes being measured.

96. The method claim 90, wherein the gene expression product is selected from the group consisting of an RNA transcript and a protein.

97. The method of claim 90, further comprising quantifying the expression level of the at least two genes or gene expression products.

98. The method of claim 97, wherein the method of quantifying the expression level of the at least two genes or gene expression products comprises the use of at least one assay selected from the group consisting of real-time quantitative PCR, microarray analysis, Nanostring, RNA sequencing, Northern blot analysis, in situ hybridisation, nCounter Analysis system analysis, or Integrated Comprehensive Droplet Digital Detection (IC 3D) analysis, and immunohistochemical analysis.

99. The method of claim 97, further comprising the step of comparing the measurement of expression of the at least two genes with a reference.

100. The method of claim 99, wherein the reference is a biological sample from a healthy patient or wherein the reference is one or more housekeeping genes.

101. The method of claim 90, wherein the biological sample is from a patient having or suspected of having cancer.

102. The method of claim 90, wherein the method comprises:
(i) providing or obtaining a patient sample;
(ii) determining the gene expression profile of the sample, wherein the gene expression profile is based on the expression the at least two genes being measured;
(iii) optionally correlating the gene expression profile of the sample to a reference; and
(iv) diagnosing or prognosing cancer in the patient.

103. The method of claim 102, further comprising assigning a therapy or therapeutic regimen to the patient.

104. The method of claim 102, wherein the method comprises determining a ratio of expression of the gene or genes positively correlated with disease score to expression of the gene or genes negatively correlated with disease score, wherein genes positively correlated are COL11A1, COMP, FN1, VCAN, CTSB and COL1A1 and genes negatively correlated with disease score are ANXA6, LGALS3, ANXA1, AB13BP, LAMB1, CTSG, LAMA4, TNXB, AGT, FBLN2, HSPG2, COL6A6, ANXA5, LAMC1, COL15A1 and VWF.

105. The method of claim 102, wherein the method comprises:
(i) determining an average level of gene expression for the genes positively correlated with disease score whose expression level is quantified;
(ii) determining an average level of gene expression for the genes negatively correlated with disease score whose expression level is quantified;
(iii) providing a matrix index, wherein the matrix index is the average level of expression of the positively correlated genes determined in step (i) divided by the average level of expression of the negatively correlated genes determined in step (ii).

106. The method of claim 102, further comprising calculating a hazard ratio from the matrix index, wherein the hazard ratio is indicative of the probability of patient survival.

107. A method of treating cancer, comprising administering a cancer therapy or initiating a therapeutic regimen for cancer if cancer is diagnosed or suspected, wherein cancer has been diagnosed or prognosed in the sample according to a method of claim 90.

108. A kit for diagnosis or prognosis of cancer, comprising means for measuring at least two genes selected from the group consisting of COL11A1, CTSB, ANXA6, LGALS3, ANXA1, AB13BP, COMP, COL1A1, LAMB1, CTSG, LAMA4, TNXB, FN1, AGT, FBLN2, HSPG2, COL6A6, VCAN, ANXA5, LAMC1, COL15A1 and VWF.

109. A microarray, comprising specific binding molecules that hybridize to an expression product from at least two genes selected from the group consisting of COL11A1, CTSB, ANXA6, LGALS3, ANXA1, AB13BP, COMP, COL1A1, LAMB1, CTSG, LAMA4, TNXB, FN1, AGT, FBLN2, HSPG2, COL6A6, VCAN, ANXA5, LAMC1, COL15A1 and VWF.
